(19) United States
(12) Patent Application Publication
(10) Pub. No.: US 2026/0091138 A1
(43) Pub. Date: Apr. 2, 2026

(54) VARIANTS OF COAGULATION FACTOR VIII AND USES THEREOF

(71) Applicant: Seattle Children’s Hospital d/b/a Seattle Children’s Research Institute, Seattle, WA (US)

(72) Inventor: Carol Hsing Miao, Seattle, WA (US)

(73) Assignee: Seattle Children’s Hospital d/b/a Seattle Children’s Research Institute, Seattle, WA (US)

(21) Appl. No.: 19/113,657

(22) PCT Filed: Sep. 20, 2023
(86) PCT No.: PCT/US2023/074704

§ 371 Date: Mar. 20, 2025

Related U.S. Application Data
(60) Provisional application No. 63/376,416, filed on Sep. 20, 2022.
(60) Provisional application No. 63/482,536, filed on Jan. 31, 2023.
(60) Provisional application No. 63/500,486, filed on May 5, 2023.

(51) Int. Cl. A61K 48/00; A61K 38/37; A61P 7/04; C07K 14/755; C12N 15/86

(52) U.S. Cl. CPC A61K 48/0058; A61K 38/37; A61P 7/04; C07K 14/755; C12N 15/86; C12N 2740/15043; C12N 2740/15045

(57) ABSTRACT

Variants of coagulation factor VIII (FVIII) and expression cassettes encoding the FVIII variants thereof are described. A variant FVIII includes a glycoepitope of the FVIII protein including an N2118Q mutation. The N2118Q mutation can be combined with other mutations including a BDD-FVIII, N6, V3, RH, furin-cleavage site deletion. X10, K12, and/or F309S mutation to form additional FVIII variants. The FVIII variants with the N2118Q mutation and expression cassettes thereof can result in reduced immunogenicity of the resulting protein. When combined with other FVIII mutations, higher gene expression, increased secretion, increased stability, and higher FVIII functional activity can be achieved by the expressed FVIII variants. The variant FVIII and expression cassettes described here can be useful in protein replacement therapy and/or gene therapy for the treatment of hemophilia A.

### CROSS-REFERENCE TO RELATED APPLICATIONS

This application is a U.S. National Phase Patent Application based on International Patent Application No. PCT/US2023/074704, filed on Sep. 20, 2023, which claims priority to U.S. Provisional Patent Application No. 63/500,486 filed May 5, 2023, U.S. Provisional Patent Application No. 63/482,536 filed Jan. 31, 2023, and U.S. Provisional Patent Application No. 63/376,416 filed Sep. 20, 2022, the entire contents of each of which are incorporated by reference herein in their entirety.

### STATEMENT REGARDING FEDERALLY SPONSORED RESEARCH OR DEVELOPMENT

This invention was made with government support under HL142019 and HL151077 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3E93267.xml. The file is 105,997 bytes, was created Mar. 13, 2025, and is being submitted electronically via Patent Center

### FIELD OF THE DISCLOSURE

The current disclosure describes variants of coagulation factor VIII (FVIII), expression cassettes encoding the FVIII variants, and uses thereof in the treatment of hemophilia A.

### BACKGROUND OF THE DISCLOSURE

Hemophilia A is a serious bleeding disorder characterized by a deficiency of the blood coagulation factor VIII (FVIII). Patients are treated acutely or prophylactically by FVIII protein replacement therapy, which is costly and inconvenient. With successful gene therapy, Hemophilia A patients would be relieved from repeated intravenous infusions of FVIII. Recent clinical trials for hemophilia A gene therapy using recombinant adeno-associated (rAAV) vectors have shown very promising results. Several other viral and nonviral gene therapy strategies have also shown promising results in preclinical models. Nevertheless, immune responses or toxic side effects induced by using high-dosage vectors remain a main hurdle for successful gene therapy.

FVIII is a complex 2,351 amino acid protein. The native amino acid sequence of FVIII is organized into six structural domains: an A1 domain, an A2 domain, a B domain, an A3 domain, a C1 domain, and a C2 domain as shown in FIG. 1A. The B domain provides 18 of 25 potential asparagine (N)-linked glycosylation sites of this protein. The B domain has no apparent function in coagulation and can be deleted with the B-domain deleted FVIII molecule still having pro-coagulatory activity.

### SUMMARY OF THE DISCLOSURE

The present disclosure describes variants of coagulation factor VIII (FVIII) and expression cassettes encoding the FVIII variants. In particular embodiments, the variant FVIII includes an N2118Q mutation within the C1 domain of the FVIII protein. This mutation results in reduced immunogenicity in subjects as compared to native FVIII. FVIII variants with an N2118 mutation can be referred to herein as 2118Q-FVIII variants.

Embodiments disclosed herein can include additional mutations that provide one or more additional administration benefits. Additional administration benefits can include increased vector packaging efficiency as compared to native FVIII, increased expression as compared to native FVIII, increased secretion as compared to native FVIII, increased stability as compared to native FVIII, or increased functional activity as compared to native FVIII. FIG. 1B provides depictions of mutations and variants that can create these various additional administration benefits.

Referring to FIG. 1B, BDD-FVIII refers to a FVIII variant with the B domain of the protein deleted (BDD=B domain deletion). BDD-FVIII has similar function compared to the native FVIII but because of its B domain deletion, it is shorter and therefore more easily packaged into vectors for delivery.

Particular embodiments utilize the following FVIII mutations to provide increased secretion compared to native FVIII:
a truncated B domain having 226 amino acids and only 6 N-linked glycosylation sites (the “N6” mutation);
a truncated B domain having 17 amino acids (the “V3” mutation);
10 mutations within the A1 domain particularly at V861, F105Y, S108A, E115D, H117Q, L129F, K132G, Q134H, T147M, and P152L (the “X10” mutation);
and/or
an F309S mutation within the 11-residue hydrophobic beta sheet within the A1 domain (the “F309S” mutation).

Particular embodiments utilize the following FVIII mutations to provide increased expression as compared to native FVIII:
the X10 mutation.

Particular embodiments utilize the following FVIII mutation to provide increased stability compared to native FVIII:
an R1645H mutation causing slower dissociation of the A2 domain (the “RH” mutation).

Particular embodiments utilize the following FVIII mutations to provide increased FVIII functional activity compared to native FVIII:
the RH mutation;
a furin cleavage site deletion (denoted as “FVIII*”);
12 mutations with the C1 and C2 domains particularly at V18571, H1859R, M1907K, M1926K, L1975V, A1993V, H2007Q, D2066E, K2085M, Q2113H, S2157N, and R2159H (the “K12” mutation).

In particular embodiments, the variant FVIII includes multiple mutations such as the FVIII variants including BDD-FVIII-N2118Q, BDD-FVIII*-N2118Q, F8/N6-N2118Q, F8/V3-N2118Q, BDD-FVIII-RH-N2118Q, F8/N6RH-N2118Q, BDD-FVIII-X10-N2118Q, F8/N6-X10-N2118Q, BDD-FVIII-X10-RH-N2118Q, F8/N6-X10-RH-N2118Q, BDD-FVIII-F309S-N2118Q, BDD-FVIII-K12-N2118Q, F8/N6K12-N2118Q, BDD-FVIII-K12-N2118Q-RH, F8/N6-K12-N2118Q-RH, BDD-FVIII-X10-K12-N2118Q, F8/N6X10-K12-N2118Q, BDD-FVIII-X10-K12-N2118Q-RH, F8/N6-X10-K12-N2118Q-RH, F8/V3X10-K12-F309S-N2118Q, BDD-FVIII*-X10-K12-F309S-N2118Q-RH, or F8/N6-X10-K12-F309S-N2118Q-RH. In particular embodiments, the variant FVIII and/or expression cassettes described herein result in higher gene expression, increased secretion, increased stability, higher FVIII functional activity, and/or reduced immunogenicity as compared to native FVIII. The variant FVIII and expression cassettes described herein can be useful in protein replacement therapy and/or gene therapy.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings. The patent or application file contains at least one drawing executed in color. Copies of this patent or patent application publication with color drawing(s) will be provided by the Office upon request and payment of the necessary fee.

FIGS. 1A-1D. (1A) Depiction of Factor VIII protein domains; (1B) depictions of Factor VIII variants that provide administration benefits; (1C) Primer sequences used in the mutagenesis experiments to convert 4 N-glycosylation sites from asparagine (N) to glutamine (Q), respectively. (1D) Coagulation factor sequences.

FIG. 2. WT BDD-FVIII plasmid used for N to Q Mutagenesis and the resulting FVIII variant constructs used for in-vivo gene therapy. The WT BDD-FVIII plasmid construct is shown on the top. The four N-glycosylation sites, N41, N239, N1810 and N2118, in the A1, A3 and C1 domains are separately represented by pins. N to Q mutagenesis was performed on the WT BDD-FVIII backbone to eliminate glycosylation on each of the four sites represented by the stop symbol. WT, wild type; BDD, B domain deletion, HCR, hepatic control region; hAAT, human α1-antitrypsin promoter; hFIXintA, human factor IX intron 1; bpA, bovine growth hormone polyadenylation site.

FIGS. 3A-3D. In-vivo experimental layout and comparison of activities of mutated FVIII variants and WT BDD-FVIII. Groups of hemophilia A (HA) mice were injected hydrodynamically with plasmids encoding WT BDD-FVIII and four N-glycosylation sites mutated BDD-FVIII variants, respectively. (3A) The experimental schedule of first and second challenges of FVIII plasmids via hydrodynamic injections and periodic blood collection for activated partial thromboplastin time (aPTT) analysis, Bethesda assay and inhibitor ELISA. (3B) FVIII activities in plasma by aPTT at 1 week post plasmid injection. p=0.95 calculated using one-way ANOVA among all plasmid treated groups. Untreated mice were used as negative controls. (3C) Specific activities of each FVIII variant compared with WT BDD-FVIII. Antigen levels of FVIII were evaluated using a FVIII-specific ELISA. N indicates the number of animals per group. (3D) FVIII activities evaluated over time. WT BDD-FVIII: N=14, N41Q: N=5, N239Q: N=5, N1810Q: N=7, N2118Q: N=6, Untreated: N=4. Experiments for each group were repeated at least 3 times. Data is presented as averages from repeated experiments with error bars indicating standard deviation.

FIGS. 4A, 4B. Inhibitor development in mice treated with plasmids carrying WT-BDD-FVIII and mutated FVIII variants. Groups of mice were injected hydrodynamically with plasmids encoding mutated FVIII variants and WT BDD-FVIII, respectively. Second challenges were performed via hydrodynamic injection in all groups on Day 86. (4A) FVIII-Specific IgG levels were analysed by ELISA. (4B) Anti-FVIII inhibitor titers were measured using Bethesda assay. Arrows indicate the time of FVIII plasmid challenges. The comparison of inhibitor titers between different treatment groups at day 100 and 107 were shown in the table. WT BDD-FVIII: N=16, N41Q: N=7, N239Q: N=6, N1810Q: N=8, N2118Q: N=6, Untreated: N=4. Experiments for each group were repeated at least 3 times. Data is presented as averages from repeated experiments with error bars indicating standard deviation. The p-values were calculated using 2-way ANOVA among all plasmid treated groups. Untreated mice were used as negative controls.

FIGS. 5A-5C. Comparison of FVIII functional activity and inhibitor development following gene transfer of AAV carrying WT-BDD-FVIII and mutated FVIII 2118Q variants. HA mice were intravenously injected with 1×10¹² vg of AAV carrying WT-BDD-FVIII and mutated FVIII 2118Q variant, respectively. (5A) Schematic of the treatment and blood collection schedule. (5B) The plasma was collected at marked timepoints for the detection of FVIII functional activity. Mice were subsequently challenged intravenously with 5 U of FVIII weekly for six weeks from week 16 to week 21. (5C) The prevalence of inhibitor development was calculated one week after final FVIII challenge. Mice had higher than 0.6 BU inhibitor titer were considered as inhibitor positive. The data are presented as means with standard deviation from three separate experiments.

FIGS. 6A-6E. CD4⁺ T-cell proliferation in response to FVIII glycosylated peptides. (6A) A graphic representation of the synthetic 15 amino-acid peptides corresponding to N-glycosylation site 2118 was shown. A non glycosylated peptide, 15 amino acids in length, centered around site N2118(2118NGP1) was modified with the addition of either a single GlcNAc residue (2118GlcP1), or a high mannose glycan Man6GlcNAc2(2118MP1). (6B) CD4⁺ T-cell proliferation levels in response to 1U FIX as a non-specific control, no stimulant as the negative control, and increasing doses of rFVIII (0.1 U and 1 U). (6C) CD4⁺ T-cell proliferation rates measured after stimulation with 2118NGP1, 2118GlcP1, and 2118MP1 synthetic peptides, respectively. (6D) Summary of the comparison of CD4⁺ T-cells proliferative rate after stimulation with different synthetic peptides from multiple experimental runs. The background was subtracted for each run. The data are presented as means with standard deviation from three separate experiments (**p<0.01, ***p<0.001, ****p<0.0001). (6E) CD4⁺ T-cell proliferation rates in response to FVIII and MP1 in the presence or absence of mannan, respectively (*p<0.05).

FIG. 7. Scatter plot to show individual data points used to prepare FIG. 6D.

FIGS. 8A, 8B. CD4⁺ T-cell proliferation in response to overlapping mannosylated peptides. (8A) Peptides, 15 amino acids in length, overlapping with 2118MP1 were synthesized with high mannose glycan Man6GlcNAc2 attachments. (8B) CD4+ T-cell proliferation levels were measured in response to mannosylated peptides MP1, MP2, and MP3 and their non-glycosylated counterparts (NGP1, NGP2, NGP3). The data are presented as means with standard deviation from three separate experiments (**p<0.01, ***p<0.001).

FIG. 9. Scatter plot to show individual data points used to prepare FIG. 8B.

FIG. 10. Glycan form by percent occupancy. * The percentage listed next to each glycan form represents how often this glycan was detected at that site. Glycan forms at each glycosylation site were previously evaluated by mass spectrometry. Glycan forms for rFVIII were isolated from Kogenate FS (baby hamster kidney-cell derived) (Bayer AG) and pdFVIII glycan forms from FVIII enriched cryoprecipitate obtained from Shanghai Lai Shi Blood Products Co., Ltd (Shanghai, China). The experiments and analyses were performed at Georgia State University. †For N2118 site, variable occupancies of mannosylation of this site have been reported. † For N2118 site, variable occupancies of mannosylation of this site have been reported. Lai et al., Haematologica. 2018; 103(11): 1925-1936; Canis K, et al., J Thromb Haemost. 2018; 16(8): 1592-1603; Qu J, et al., PLoS One. 2020; 15(5): e0233576.

FIG. 11. Homology of FVIII peptides representative of site N2118. The amino acid sequences of 2118MP1, 2118MP2 and 2118MP3 were compared between the homologous sequences in human, mouse, rat, pig, and dog species.

FIG. 12. Peptides of the three non-glycosylated peptides (NGP) are shown.

FIGS. 13A-13D. Mouse Surgery Strategy and transducer design. (13A) Schematic of the ultrasound-mediated gene delivery (UMGD) procedure, where a midline incision is made, and a catheter is inserted into the portal vein. A microbubble (MB)/plasmid solution (2.5 mg plasmid/kg and 1.25 ml MBs/kg) is injected over 30 seconds while the ultrasound transducer is placed on the liver surface for 1 minute of treatment. (13B) H158-002 is a single-element, unfocused transducer with a diameter of 16 mm with the sound pressure profile as measured in degassed water is plotted. (13C) A pressure map of H158 is shown with X and Y axes plotted against relative pressure. (13D) A pulse train method of ultrasound treatment is employed, where the ultrasound power is alternated between on and off in order to achieve high levels of transfection with low levels of damage to the liver.

FIGS. 14A, 14B. First step in targeting liver sinusoidal endothelial cells (LSECs), was to determine what ultrasound (US) conditions primarily targeted endothelial cells. To do this, a matrix of varying powers and pulse durations was created (FIG. 14A) and mice were transfected with a GFP reporter plasmid (FIG. 14B). From this, it was determined that 50W power with 150 us pulse duration, was a condition that showed the most endothelial only cell transfection. This confirmed the hypothesis that a lower power would be able to target endothelial cells, in comparison to a previously used hepatocyte condition of 100W, 150 μs.

FIGS. 15A, 15B. Ultrasound condition matrix using GFP reporter plasmid. (15A) BL6 mice were exposed to a matrix of US conditions for a total treatment time of 1 minute. Mice were sacrificed at 24 hours; livers were sectioned at 7 μm and transfection was examined using a fluorescent microscope following immunofluorescent staining with anti-GFP and anti-LYVE-1 on a fluorescent microscope at 10×. An untreated mouse is shown for comparison (ix in FIG. 15B). (15B) UMGD US condition matrix and subsequent transfection image patterns.

FIGS. 16A-16D. Assessment of ubiquitous chromatin opening element (UCOE)-ICAM2-F8N6X10 plasmid in mice. (16A) Construct of the UCOE-ICAM2-F8N6X10, ICAM2-F8N6X10, and pHP-hF8 plasmids. (16B) Three groups of HA mice were hydrodynamically injected with one of three plasmids: UCOE-ICAM2-F8N6X10, ICAM2-F8N6X10, and pHP-hF8. FVIII functional activity levels were measured on days 1 and 7 following injection. Liver tissue collected one week after hydrodynamic injection with UCOE-ICAM2-F8N6X10 or pHP-hF8 was subjected to RNAscope (16C) and immunofluorescent staining (16D). mRNA in RNAscope imaging is stained blue for DAPI, green for hFVIII, and red for Lyve-1. Protein in immunofluorescent imaging is stained blue for DAPI, green for hFVIII, and red for CD-31. Colocalization between hFVIII and endothelial markers appears yellow and are pointed out with arrows.

FIGS. 17A, 17B. Comparison of FVIII expression and inhibitor formation in low energy (LE) & high energy (HE) groups over 84 days. (17A) Using UMGD, the UCOE-ICAM2-F8N6X10 plasmid was delivered in BL6/HA mice using either LE (50W, 150 μs), n=17, or HE conditions (100W, 150 μs), n=14. Blood samples were collected and subjected to the aPTT assay at various time points through D84. (17B) The Bethesda assay was run on blood samples to determine FVIII inhibitor levels. Five of ten HE mice developed measurable inhibitors, while none of the fourteen LE mice had detectable inhibitors.

FIGS. 18A-18D. Presence and location of UCOE-ICAM-F8N6X10 mRNA in treated mice liver tissue. Mouse liver was transfected using the UCOE-ICAM-F8N6X10 plasmid via UMGD, and livers were collected at an early (day 7) and late time point (day 120). These livers were sectioned and stained using RNAscope protocols (18A) In the images, UCOE-ICAM-F8N6X10 derived mRNA, shown in green, colocalized with the Lyve-1 endothelial marker, shown in red, during day 7 and at D120 in LE mice. Liver harvested from HE mice on day 7 and day 120 shows fewer regions of colocalization. (18B) Livers collected from LE & HE mice at D180 were stained using immunofluorescent protocols, with DAPI shown in blue, hFVIII in green, and CD-31 in red. LE mice exhibit colocalization between hFVIII and CD-31 while HE mice do not. Livers from LE mice harvested at day 7 (18C) and day 120 (18D) showed colocalized expression of hFVIII mRNA shown in green, and Lyve-1 mRNA in red.

FIGS. 19A, 19B. Determination of potential damage to the liver following UMGD. (19A) The alanine transaminase or ALT assay were run on blood samples from the mice in both groups for 2 weeks. Normal ranges of untreated mice fall between 5 and 60, shown by the grey bar on the graph. Both groups of mice show a peak outside of normal ranges at D1 post-surgery, however then return to normal values by D3. LE condition mice have much lower initial damage, though they are comparable to the high energy mice for the remainder of the time. (19B) D7 histology show the HE mice have more inflammation and transient damage initially, however, both mice returned to the control level of damage by D120.

FIGS. 20A, 20B. Determination of potential damage to the liver following UMGD. (20A) Blood samples were collected from the mice following UMGD to assess levels of alanine transaminase as markers for liver damage. The normal range for mouse ALT (18-65 IU/L) is highlighted in grey. (20B) HE, LE, and a control mouse were sacrificed on day 7 and day 120. Liver sections were fixed in 10% formalin, paraffinized, stained with hematoxylin & eosin, and imaged at 10×. Representative images of maximum damage are shown.

FIG. 21. FVIII plasmid optimization schematic.

FIGS. 22A, 22B. Additional Immunostaining of LE Mice following UMGD of GFP Reporter plasmid. Mice treated with the 50W, 150 μs US condition to deliver the GFP reporter plasmid were sacrificed 24 hours after treatment. Their livers were collected, sectioned, and transfection was examined using a fluorescent microscope following immunofluorescent staining. (22A) Two different liver sections from the same mouse were stained with anti-GFP (green) and anti-LYVE-1 (red). The left image showed staining of both endothelial transfection patterns with colocalization with Lyve-1 on the right part, and hepatic transfection patterns on the left part. The right image showed evidence of colocalization with Lyve-1, but with much lower levels of transfection with the GFP plasmid. (22B) This liver section was stained with anti-GFP (green) and anti-CD31 (red) and showed very high levels of colocalization between GFP and CD-31, with little GFP that was not overlapped with CD-31.

FIGS. 23A, 23B. Analysis of cell type transfection utilizing alternative RNAscope staining. Mouse liver was collected from HE and LE UMGD treated mice on day 120. Livers were sectioned and stained using RNAscope protocols. (23A) In these images, UCOE-ICAM-F8N6X10 derived mRNA is shown in green, Lyve-1 in red, albumin in cyan, and DAPI in blue. In the LE group of mice, there are high levels of colocalization between hFVIII and Lyve-1 as indicated by the yellow arrows, and no visible colocalization between hFVIII and albumin. In the HE mice, there is a small region of colocalization between hFVIII and Lyve-1 as indicated by the yellow arrow, in addition to colocalization between hFVIII and albumin as indicated by the white arrow. (23B) In these images, UCOE-ICAM-F8N6X10 derived mRNA is shown in green, Lyve-1 in red, CD-31 in cyan, and DAPI in blue. The LE mice display colocalization of hFVIII with both CD-31, as pointed out by the white arrows, and Lyve-1, as indicated by the yellow arrows. The group of HE mice have less instances of colocalization with either, though a small overlap between hFVIII and CD-31 is indicated by the white arrow.

FIG. 24. Sequences supporting the disclosure including: BDD-F8 (SEQ ID NO: 21), BDD-F8-K12 (SEQ ID NO: 22), BDD-F8-N2118Q (SEQ ID NO: 23), BDD-F8-RH (SEQ ID NO: 24), BDD-F8-V3 (SEQ ID NO: 25), BDD-F8-X10 (SEQ ID NO: 26), BDD-F8-X10-N2118Q (SEQ ID NO: 27), F8-N6 (SEQ ID NO: 28), F8-N6-K12 (SEQ ID NO: 29), F8-N6-K12-RH (SEQ ID NO: 30), F8-N6-N2118Q (SEQ ID NO: 31), F8-N6-RH (SEQ ID NO: 32), F8-N6-X10 (SEQ ID NO: 33), F8-N6-X10-N2118Q (SEQ ID NO: 34), and BDD-cF8-X10-N2118Q (SEQ ID NO: 35).

### DETAILED DESCRIPTION

Hemophilia A is a serious bleeding disorder characterized by a deficiency of the blood coagulation factor VIII (FVIII). The Factor VIII gene located on the X chromosome is large and structurally complex, including 180 kb and 26 exons. The wild-type Factor VIII gene encodes two proteins. The first protein is the full-length Factor VIII protein, which is encoded by the 9030 bases found in exons 1 to 26 and has a circulating form containing 2332 amino acid residues. The second protein, referred to as Factor VIIIb, is encoded by 2598 bases in 5 exons present in the Factor VIII gene. The resulting protein includes 216 amino acids and has a presently unknown function. Hemophila A is associated with large deletions, insertions, inversions, and point mutations within the Factor Vill gene. In particular embodiments, Factor VIII in humans includes the sequence as set forth in SEQ ID NO: 13.

Patients are treated acutely or prophylactically by protein replacement therapy, which is costly and inconvenient. With successful gene therapy, Hemophilia A patients would be relieved from repeated intravenous infusions of FVIII. Recent clinical trials for hemophilia A gene therapy using recombinant adeno-associated (rAAV) vectors have shown very promising results. Several other viral and nonviral gene therapy strategies have also shown promising results in preclinical models. Nevertheless, immune responses or toxic side effects induced by using high-dosage vectors remain a main hurdle for successful gene therapy. Methods and systems for high level gene expression and/or reduced immunogenicity of FVIII variants are needed to reduce vector doses required to achieve a therapeutic effect.

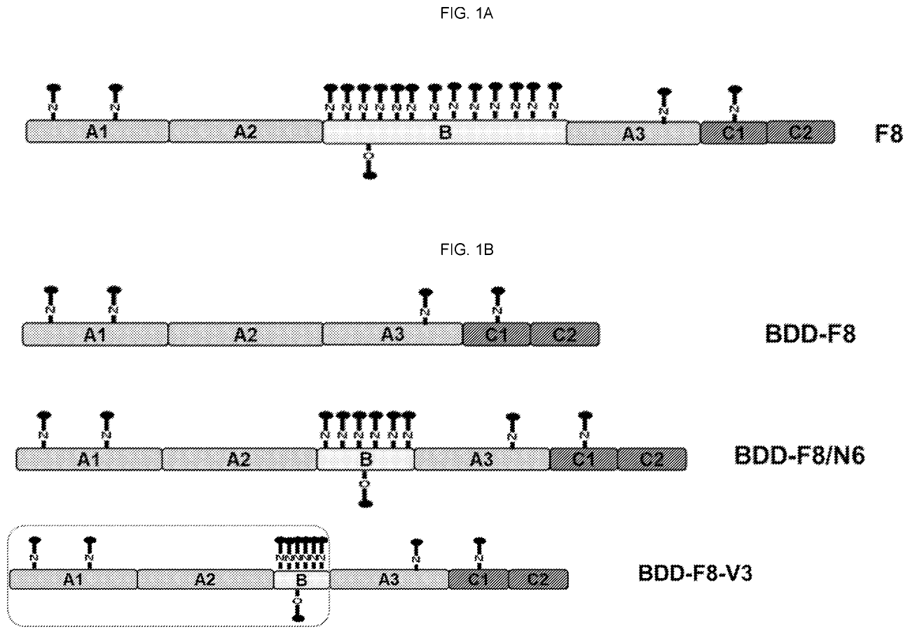

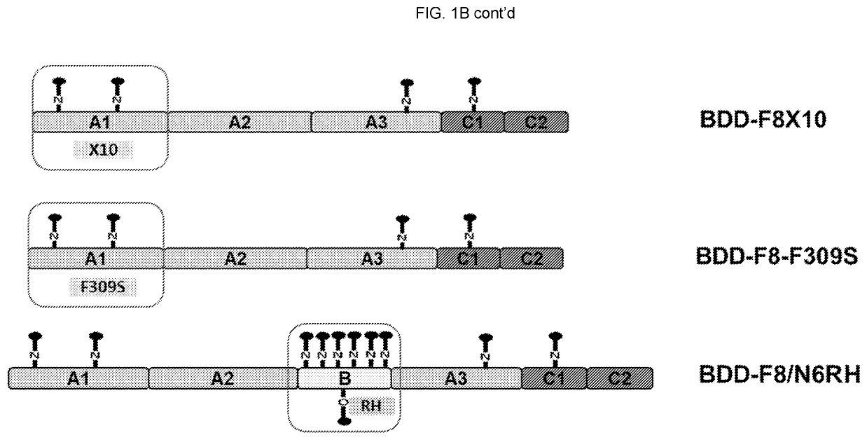

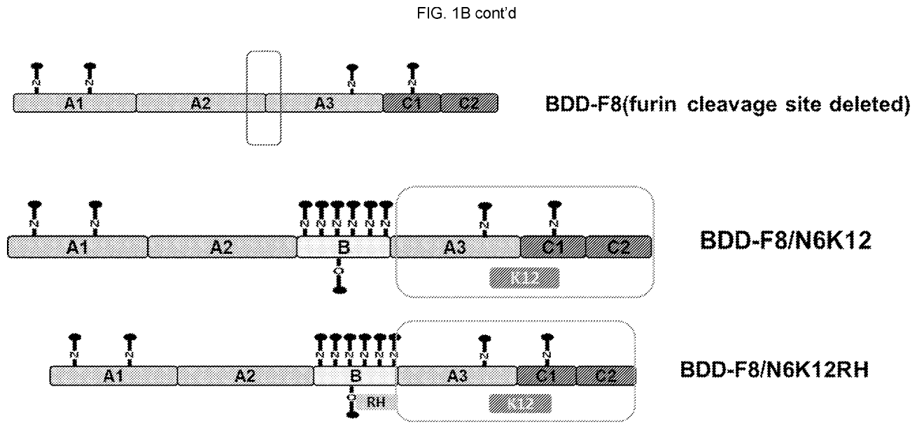

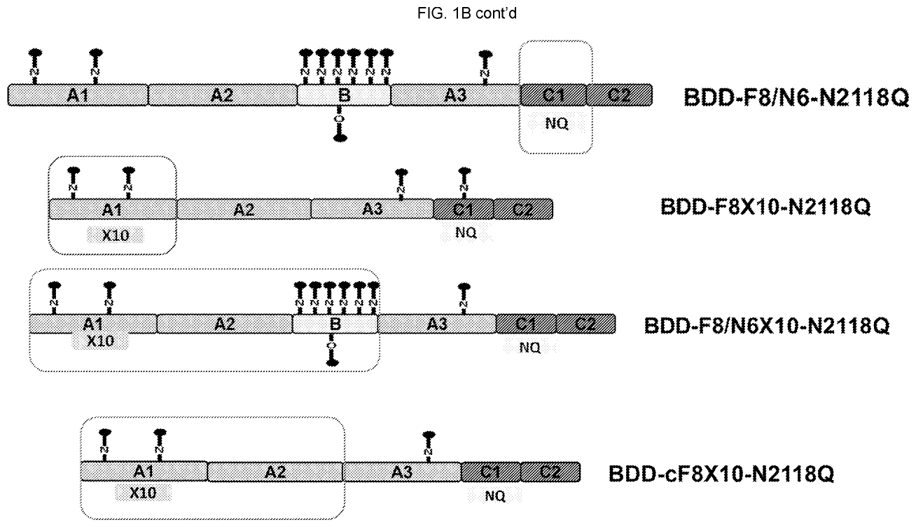

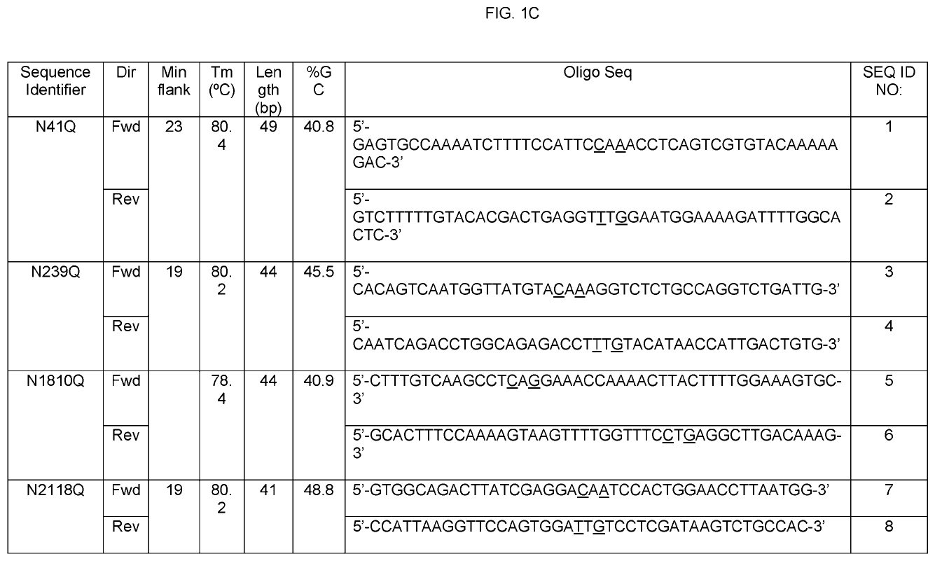

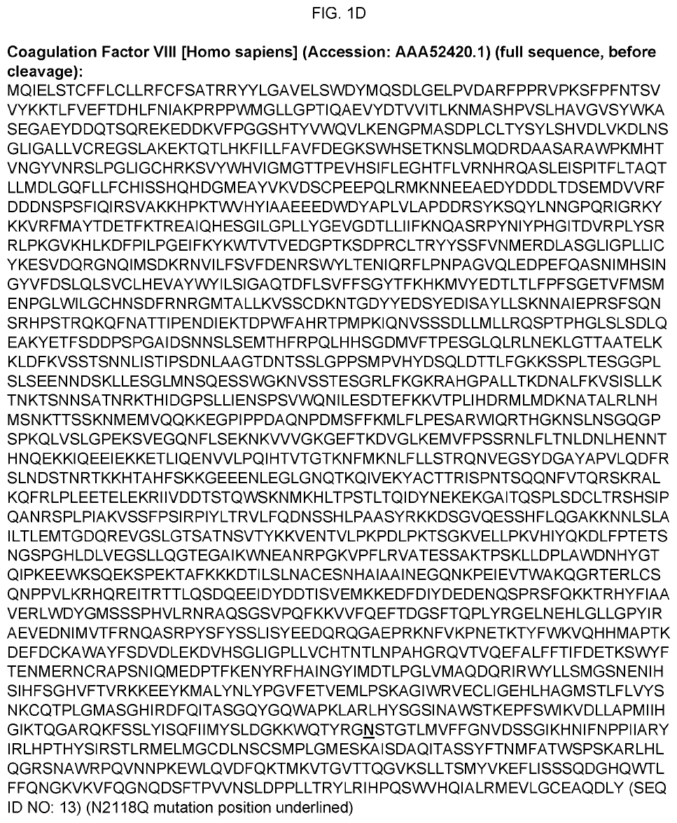

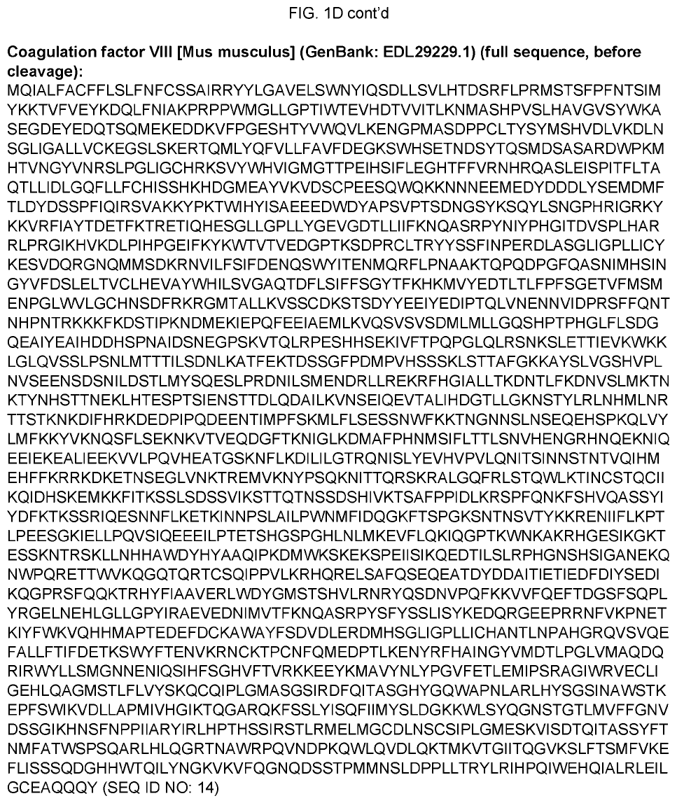

The present disclosure describes variants of coagulation factor VIII (FVIII) and expression cassettes encoding the FVIII variants. In particular embodiments, the variant FVIII is a glycoepitope of the FVIII protein including an N2118Q mutation, herein referred to as a 2118Q-FVIII variant. In particular embodiments, the 2118Q-FVIII variant produces increased FVIII functional activity and reduced immunogenicity as compared to native FVIII. Embodiments disclosed herein can include additional mutations that provide one or more additional administration benefits. In particular embodiments, the variant FVIII includes a mutated FVIII with a deleted B domain, referred to as BDD-FVIII. The BDD-FVIII has similar function compared to the native FVIII but because of its B domain deletion, it is shorter and therefore more easily packaged into vectors for delivery. Particular embodiments utilize the following FVIII mutations to provide increased secretion compared to native FVIII: a N6 mutation includes a 226 aa B-domain variant sequence such that the B-domain only has 6 N-linked glycosylation sites; a V3 mutation includes a 17-aa peptide to replace the B domain; an X10 mutation includes 10 mutations with the A1 domain particularly at V861, F105Y, S108A, E115D, H117Q, L129F, K132G, Q134H, T147M, and P152L; and/or an F309S mutation includes an F309S mutation within the 11-residue hydrophobic beta sheet within the A1 domain. Particular embodiments utilize the RH FVIII mutation to provide increased stability compared to native FVIII, wherein the RH mutation includes an R1645H mutation causing slower dissociation of the A2 domain. Particular embodiments utilize the following FVIII mutations to provide increased FVIII functional activity compared to native FVIII: the RH mutation; a furin cleavage site deletion (FVIII*); and/or a K12 mutation, wherein a K12 mutation includes 12 mutations with the C1 and C2 domains particularly at V18571, H1859R, M1907K, M1926K, L1975V, A1993V, H2007Q, D2066E, K2085M, Q2113H, S2157N, and R2159H. Particular embodiments use the X10 mutation as described earlier to enhance expression compared to native FVIII. See FIG. 1B for a schematic of each FVIII variant.

In particular embodiments, the variant FVIII includes multiple mutations such as the FVIII variants including BDD-FVIII-N2118Q, BDD-FVIII*-N2118Q, F8/N6-N2118Q, F8/V3-N2118Q, BDD-FVIII-RH-N2118Q, F8/N6RH-N2118Q, BDD-FVIII-X10-N2118Q, F8/N6-X10-N2118Q, BDD-FVIII-X10-RH-N2118Q, F8/N6-X10-RH-N2118Q, BDD-FVIII-F309S-N2118Q, BDD-FVIII-K12-N2118Q, F8/N6K12-N2118Q, BDD-FVIII-K12-N2118Q-RH, F8/N6-K12-N2118Q-RH, BDD-FVIII-X10-K12-N2118Q, F8/N6X10-K12-N2118Q, BDD-FVIII-X10-K12-N2118Q-RH, F8/N6-X10-K12-N2118Q-RH, F8/V3X10-K12-F309S-N2118Q, BDD-FVIII*-X10-K12-F309S-N2118Q-RH, or F8/N6-X10-K12-F309S-N2118Q-RH, wherein the * indicates a furin cleavage site deletion.

In particular embodiments, the variant FVIII is a canine FVIII (cFVIII) variant. In particular embodiments, the cFVII includes a mutated cFVIII with a deleted B domain, referred to as BDD-FVIII. In particular embodiments, the cFVIII includes an N2118Q mutation. In particular embodiments, the cFVIII includes an X10 mutation. In particular embodiments, the cFVIII cFVIII includes multiple mutations such as the cFVIII variants including BDD-cF8-X10-N2118Q.

In particular embodiments, a variant FVIII is encoded by a sequence as set forth in any one of SEQ ID NOs: 21-35. In particular embodiments, if not present, a coding sequence is altered to encode an N2118Q mutation within the variant FVIII. For example, SEQ ID NO: 21, 22, 24-26, 28-30, 32, or 33 could be altered to encode an N2118Q mutation. Codons that encode N include AAT and AAC, which can be deleted from coding sequences, while codons that encode Q include CAA and CAG, which can be incorporated into coding sequences, as is understood by one of ordinary skill in the art. In particular embodiments, the variant FVIII is encoded by a sequence as set forth in SEQ ID NO: 23, 27, 31, 34, or 35. The FVIII variants and expression cassettes described here can be useful in protein replacement therapy and/or gene therapy. Particular expression cassettes encoding FVIII variants can be further codon optimized or designed for liver sinusoidal endothelial cells (LSECs)-specific expression, hepatic specific expression, and ubiquitous expression.

Aspects of the current disclosure are now described in more supporting detail as follows: (i) Factor VIII Proteins and Variants; (ii) Genetic Constructs; (iii) Vectors; (iv) Targeted Genetic Engineering; (v) Nanoparticles and Microbubbles; (vi) Compositions for Administration; (vii) Methods of Use; (viii) Exemplary Embodiments; (ix) Experimental Examples; and (x) Closing Paragraphs. These headings are provided for organizational purposes only and do not limit the scope or interpretation of the disclosure.

(i) Factor VIII Proteins and Variants. Factor VIII (FVIII) is a blood plasma glycoprotein of 260 kDa molecular mass, produced in the liver of mammals. It is a critical component of the cascade of coagulation reactions that lead to blood clotting. Within this cascade is a step in which factor IXa, in conjunction with FVIII, converts factor X to an activated form (FXa). The most common hemophilic disorder is caused by a deficiency of functional FVIII called hemophilia A.

An important advance in the treatment of hemophilia A has been the isolation of cDNA clones encoding the complete 2,351 amino acid sequence of human FVIII (U.S. Pat. No. 4,757,006) and the provision of the human FVIII gene DNA sequence and recombinant methods for its production. Analysis of the deduced primary amino acid sequence of human FVIII determined from the cloned cDNA indicates that it is a heterodimer processed from a larger precursor polypeptide. The heterodimer includes a C-terminal light chain of 80 kDa in a metal ion-dependent association with a 210 kDa N-terminal heavy chain fragment. (See review by Kaufman, Transfusion Med. Revs. 6:235(1992)). Physiological activation of the heterodimer occurs through proteolytic cleavage of the protein chains by thrombin. Thrombin cleaves the heavy chain to a 90 kDa protein, and then to 54 kDa and 44 kDa fragments. Thrombin also cleaves the 80 kDa light chain to a 72 kDa protein. It is the latter protein, and the two heavy chain fragments (54 kDa and 44 kDa above), held together by calcium ions, that constitute active FVIII. Inactivation occurs when the 72 kDa and 54 kDa proteins are further cleaved by thrombin, activated protein C or FXa.

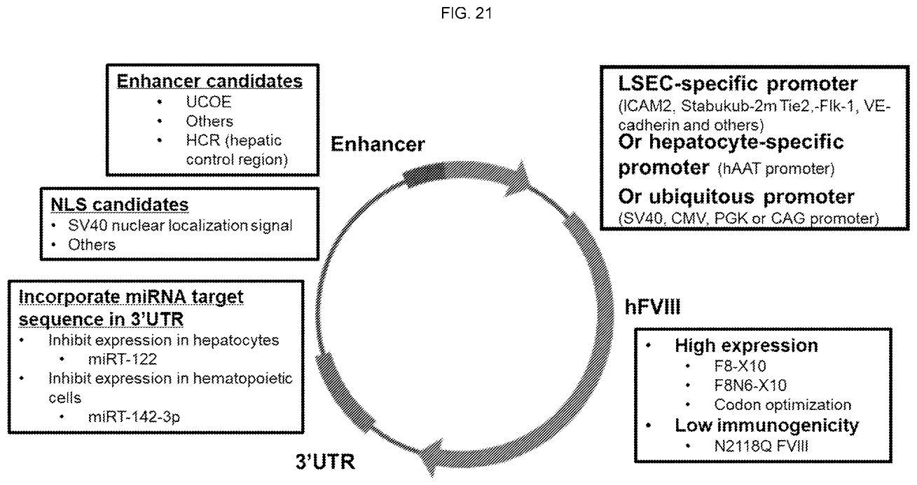

The amino acid sequence of FVIII is organized into multiple structural domains designated A1-A2-B-A3-C-C2. FIG. 21 shows a schematic of the structure of native FVIII (F8). The B domain of FVIII has no homology to other proteins and provides 18 of the 25 potential asparagine (N)-linked glycosylation sites of this protein. The B domain has no apparent function in coagulation and can be deleted with the B-domain deleted FVIII molecule (BDD-FVIII) still having procoagulatory activity.

Herein, native Factor Vill or native FVIII refers to any FVIII molecule that is in its natural state or in the state in which it would be found purified from a natural source. The FVIII molecule includes full-length native FVIII. The native FVIII protein can be derived from human plasma or be produced by recombinant engineering techniques.

Exemplary native human and murine FVIII protein sequences are provided in FIG. 1 as SEQ ID NO: 13 and SEQ ID NO: 14, respectively. As will be understood by one of ordinary skill in the art, the FVIII protein is synthesized as a single chain polypeptide of 2351 amino acids. A 19-amino acid signal peptide is cleaved by a protease shortly after synthesis so that circulating plasma factor VIII is a 2332 amino acid heterodimer. The provided GenBank sequences are numbered using the total protein (2351 aa). The numbering used in this application, outside of reference to the GenBank sequences, uses numbering from the mature protein (2332 aa without the 5′-end signal peptide). For comparison, the N2118 position in the mature/cleaved protein is at position N2137 in the total (pre-cleaved) protein. Similar adjustments can be accounted for by other residue positioning described herein. For example, residue numbering can change based on truncations N-terminal to position 2118 or 2137. One of ordinary skill in the art can account for such adjustments to residue numbering, as appropriate.

FVIII variants or variants of FVIII refer to peptides or sequences encoding the peptides including at least a portion of the sequence corresponding to a region of the FVIII molecule. In some embodiments, a FVIII variant can include a sequence identical to the particular region of a native FVIII protein. In other embodiments, a FVIII variant can be a conservatively modified variant of a region of native FVIII protein. In particular embodiments, a FVIII variant can be characterized by a certain percent identity, e.g., 85% identical, relative to the sequence of a region of native FVIII protein.

The term FVIII protein can refer to either a native FVIII or any of the variants of FVIII disclosed herein. In particular embodiments, FVIII is a full length human FVIII.

Since the size of a full-length human FVIII cDNA is quite large (>7 kbp), it cannot be easily packaged in viral vectors, often resulting in low titers of viruses. Shorter cDNAs coding for FVIII variants have therefore been used in gene therapy preclinical research. For instance, a B-domain deleted FVIII (BDD-FVIII) variant exhibits similar FVIII functional activity as the full-length native FVIII. In particular embodiments, BDD-FVIII includes a B-domain deleted human FVIII. In particular embodiments, BDD-FVIII is encoded by the sequence as set forth in SEQ ID NO: 21.

N6 (also referred to as BDD-F8/N6 or F8/N6) is a FVIII variant with a 226 aa B-domain variant sequence (Miao et al., Blood 103:3412-3419, 2004) In particular embodiments, nucleic acids encoding F8/N6 are codon-optimized (Ward et al., Blood 117:798-807, 2011). In particular embodiments, N6 results in increased secretion as compared to BDD-FVIII. In particular embodiments, BDD-F8/N6 includes a human FVIII variant with a 226 aa B-domain. In particular embodiments, BDD-F8/N6 is encoded by the sequence as set forth in SEQ ID NO: 28.

V3 (also referred to as BDD-F8/V3 or F8-V3) has a 17 aa peptide coding sequence replacing the 226 aa sequence in F8/N6 (McIntosh et al., Blood 121:3335-3344, 2013). In particular embodiments, V3 results in increased secretion as compared to BDD-FVIII. In particular embodiments, BDD-F8/V3 includes a human FVIII variant that replaces the 226 aa N6 spacer with a 17 aa peptide. In particular embodiments, BDD-F8/V3 is encoded by the sequence as set forth in SEQ ID NO: 25.

RH (also referred to as F8-RH) is an FVIII variant with an R1645H mutation (Siner et al., Blood 121:4396-4403, 2013). In particular embodiments, RH results in increased secretion compared to BDD-FVIII. In particular embodiments, RH results in a more stable FVIII single chain molecule as compared to native FVIII. In particular embodiments, RH results in increased FVIII functional activity because of a slower dissociation of the A2-domain upon thrombin activation. In particular embodiments, BDD-FVIII-RH includes a human BDD-FVIII variant with an R1645H mutation that eliminates the furin cleavage site for generating a more stable FVIII single chain molecule. In particular embodiments, BDD-FVIII-RH is encoded by the sequence as set forth in SEQ ID NO: 24. In particular embodiments, BDD-F8/N6-RH includes a human BDD-F8/N6 variant with an R 1645H mutation that eliminates the furin cleavage site for generating a more stable FVIII single chain molecule. In particular embodiments, BDD-F8/N6-RH is encoded by the sequence as set forth in SEQ ID NO: 32.

Furin-cleavage site deleted BDD-FVIII variants (Nguyen et al., J Thromb Haemostas. 15:110-121, 2017) exhibit an increase in FVIII functional activity compared with BDD-FVIII, likely due to its slower dissociation of the A2-domain upon thrombin activation.

X10 is a FVIII variant including mutations in the A1 domain, particularly at V861, F105Y, S108A, E115D, H117Q, L129F, K132G, Q134H, T147M, and P152L. In particular embodiments, X10 results in enhanced expression and/or secretion as compared to native FVIII. In particular embodiments, BDD-FVIII-X10 includes a human BDD-FVIII variant with a deleted B-domain and mutations in the A1 domain to enhance expression and/or secretion. In particular embodiments, BDD-FVIII-X10 is encoded by the sequence as set forth in SEQ ID NO: 26. In particular embodiments, BDD-F8/N6-X10 includes a human BDD-F8/N6 variant and mutations in the A1 domain to enhance expression and/or secretion. In particular embodiments, BDD-F8/N6-X10 is encoded by the sequence as set forth in SEQ ID NO: 33.

K12 is a FVIII variant including mutation in the C1 and C2 domains, particularly at V18571, H1859R, M1907K, M1926K, L1975V, A1993V, H2007Q, D2066E, K2085M, Q2113H, S2157N, and R2159H. In particular embodiments, K12 results in increased FVIII functional activity as compared to native FVIII. In particular embodiments, BDD-FVIII-K12 includes a human BDD-FVIII variant that contains mutations in C1 and C2 domains to increase functional activity. In particular embodiments, BDD-FVIII-K12 is encoded by the sequence as set forth in SEQ ID NO: 22. In particular embodiments, BDD-F8/N6-K12 includes a human BDD-F8/N6 variant that contains mutations in C1 and C2 domains to increase functional activity. In particular embodiments, BDD-F8/N6-K12 is encoded by the sequence as set forth in SEQ ID NO: 29. In particular embodiments, BDD-F8/N6-K12-RH includes a human BDD-F8/N6 variant that contains mutations in C1 and C2 domains to increase functional activity and an R1645H mutation that eliminates the furin cleavage site for generating a more stable FVIII single chain molecule. In particular embodiments, BDD-F8/N6-K12-RH is encoded by the sequence as set forth in SEQ ID NO: 30.

N41, N239, N1810, and N2118Q are FVIII variants that introduce glycosylation mutation sites outside the B domain of FVIII. For example, N2118Q (also referred to as 2118Q-FVIII) is a FVIII variant including an N2118Q mutation in the C1 domain that eliminates the 2118 glycosylation sites. In particular embodiments, N2118Q results in a less immunogenic peptide. In particular embodiments, BDD-FVIII-N2118Q includes a human BDD-FVIII variant with N2118Q mutation that eliminates the 2118 glycosylation sites to generate a less immunogenic FVIII molecule. In particular embodiments, BDD-FVIII-N2118Q is encoded by the sequence as set forth in SEQ ID NO: 23. In particular embodiments, BDD-F8/N6-N2118Q includes a human BDD-FVIII variant with an N2118Q mutation and N6 mutations to generate high expression of a less immunogenic FVIII molecule. In particular embodiments, BDD-F8/N6-N2118Q is encoded by the sequence as set forth in SEQ ID NO: 31. In particular embodiments, BDD-FVIII-X10-N2118Q includes a human BDD-FVIII variant with N2118Q mutation and X10 mutations to generate high expression of a less immunogenic FVIII molecule. In particular embodiments, BDD-FVIII-X10-N2118Q is encoded by the sequence as set forth in SEQ ID NO: 27. In particular embodiments, BDD-F8/N6-X10-N2118Q includes a human BDD-FVIII variant with N2118Q mutation, N6 mutation, and X10 mutations to generate high expression of a less immunogenic FVIII molecule. In particular embodiments, BDD-F8/N6-X10-N2118Q is encoded by the sequence as set forth in SEQ ID NO: 34. In particular embodiments, BDD-cF8-X10-N2118Q includes a canine BDD-FVIII variant with N2118Q mutation and X10 mutations to generate high expression of a less immunogenic cFVIII. In particular embodiments, BDD-cF8-X10-N2118Q is encoded by the sequence as set forth in SEQ ID NO: 35.

F309S refers to a single residue mutation, F309S, within the 11-residue hydrophobic beta-sheet within the A1 domain of FVIII. In particular embodiments, this FVIII variant increases FVIII secretion and reduces the ATP requirement for secretion as compared to native FVIII.

In particular embodiments, mutations to make a FVIII variant can be combined to make additional FVIII variants. In particular embodiments, additional FVIII variants include an N2118Q mutation including: BDD-FVIII-N2118Q, BDD-FVIII*-N2118Q, F8/N6-N2118Q, F8/V3-N2118Q, BDD-FVIII-RH-N2118Q, BDD-FVIII*-RH-N2118Q, F8/N6RH-N2118Q, F8/V3RH-N2118Q, BDD-FVIII-X10-N2118Q, F8/N6-X10-N2118Q, BDD-FVIII*-X10-N2118Q, F8/V3X10-N2118Q, BDD-FVIII-X10-RH-N2118Q, BDD-FVIII*-X10-RH-N2118Q, F8/N6-X10-RH-N2118Q, F8/V3-X10-RH-N2118Q, BDD-FVIII-F309S-N2118Q, BDD-FVIII*-F309S-N2118Q, F8/N6F309S-N2118Q, F8/V3F309S-N2118Q, BDD-FVIII-F309S-N2118Q-RH, BDD-FVIII*-F309S-N2118Q-RH, F8/N6-F309S-N2118Q-RH, F8/V3-F309S-N2118Q-RH, BDD-FVIII-K12-N2118Q, BDD-FVIII*-K12-N2118Q, F8/N6K12-N2118Q, F8/V3K12-N2118Q, BDD-FVIII-K12-N2118Q-RH, BDD-FVIII*-K12-N2118Q-RH, F8/N6-K12-N2118Q-RH, F8/V3-K12-N2118Q-RH, BDD-FVIII-X10-F309S-N2118Q, BDD-FVIII*-X10-F309S-N2118Q, F8/N6X10-F309S-N2118Q, F8/V3X10-F309S-N2118Q, BDD-FVIII-X10-F309S-N2118Q-RH, BDD-FVIII*-X10-F309S-N2118Q-RH, F8/N6-X10-F309S-N2118Q-RH, F8/V3-X10-F309S-N2118Q-RH, BDD-FVIII-X10-K12-N2118Q, BDD-FVIII*-X10-K12-N2118Q, F8/N6X10-K12-N2118Q, F8/V3X10-K12-N2118Q, BDD-FVIII-X10-K12-N2118Q-RH, BDD-FVIII*-X10-K12-N2118Q-RH, F8/N6-X10-K12-N2118Q-RH, F8/V3-X10-K12-N2118Q-RH, BDD-FVIII-K12-F309S-N2118Q, BDD-FVIII*-K12-F309S-N2118Q, F8/N6K12-F309S-N2118Q, F8/V3K12-F309S-N2118Q, BDD-FVIII-K12-F309S-N2118Q-RH, BDD-FVIII*-K12-F309S-N2118Q-RH, F8/N6-K12-F309S-N2118Q-RH, F8/V3-K12-F309S-N2118Q-RH, BDD-FVIII-X10-K12-F309S-N2118Q, BDD-FVIII*-X10-K12-F309S-N2118Q, F8/N6X10-K12-F309S-N2118Q, F8/V3X10-K12-F309S-N2118Q, BDD-FVIII-X10-K12-F309S-N2118Q-RH, BDD-FVIII*-X10-K12-F309S-N2118Q-RH, F8/N6-X10-K12-F309S-N2118Q-RH, or F8/V3-X10-K12-F309S-N2118Q-RH, wherein * indicates a furin cleavage site deletion.

In particular embodiments, additional FVIII variants include: BDD-FVIII*-RH, F8/N6RH, F8/V3RH, BDD-FVIII*X10, F8/N6X10, F8/V3X10, BDD-FVIII-X10RH, BDD-FVIII*-X10RH, F8/N6X10RH, F8/V3X10RH, BDD-FVIII*-F309S, F8/N6-F309S, F8/V3-F309S, BDD-FVIII-RH-F309S, BDD-FVIII*RH-F309S, F8/N6RH-F309S, F8/V3RH-F309S, BDD-FVIII-K12, BDD-FVIII*K12, F8/V3K12, BDD-FVIII-K12RH, BDD-FVIII*-K12RH, F8/V3K12RH, BDD-FVIII-X10-F309S, BDD-FVIII*-X10-F309S, F8/N6X10-F309S, F8/V3X10-F309S, BDD-FVIII-X10-F309S-RH, BDD-FVIII*-X10-F309S-RH, F8/N6-X10-F309S-RH, F8/V3-X10-F309S-RH, BDD-FVIII*-X10-K12, F8/N6X10-K12, F8/V3X10-K12, BDD-FVIII-X10-K12-RH, BDD-FVIII*-X10-K12-RH, F8/N6-X10-K12-RH, F8/V3-X10-K12-RH, BDD-FVIII-K12-F309S, BDD-FVIII*-K12-F309S, F8/N6K12-F309S, F8/V3K12-F309S, BDD-FVIII-K12-F309S-RH, BDD-FVIII*-K12-F309S-RH, F8/N6-K12-F309S-RH, F8/V3-K12-F309S-RH, BDD-FVIII-X10-K12-F309S, BDD-FVIII*-X10-K12-F309S, F8/N6X10-K12-F309S, F8/V3X10-K12-F309S, BDD-FVIII-X10-K12-F309S-RH, BDD-FVIII*-X10-K12-F309S-RH, F8/N6-X10-K12-F309S-RH, F8/V3-X10-K12-F309S-RH, wherein * indicates a furin cleavage site deletion.

In particular embodiments, expression constructs encoding a FVIII variant can be codon optimized.

(ii) Genetic Constructs. Desired genes encoding variant Factor VIII can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, mammalian artificial chromosomes (Vos, 1998, Curr. Op. Genet. Dev. 8:351-359), liposomes (Tarahovsky and Ivanitsky, 1998, Biochemistry (Mosc) 63:607-618), ribozymes (Branch and Klotman, 1998, Exp. Nephrol. 6:78-83), triplex DNA (Chan and Glazer, 1997, J. Mol. Med. 75:267-282), etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressed by the cell and, in certain instances, preferably heritable and expressed in its cell progeny.

The term “genetic construct” refers to a polynucleotide vehicle to introduce genetic material into a cell. In particular embodiments, the term genetic construct includes plasmids and vectors. Plasmids can be linear or circular. In particular embodiments, a genetic construct of the disclosure is circular and is linearized through action of the gene-editing components encoded on the genetic construct. Genetic constructs can include, for example, an origin of replication, a multicloning site, and/or a selectable marker. An expression genetic construct typically includes an expression cassette. The term “expression cassette” includes a polynucleotide construct that is generated recombinantly or synthetically and includes regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell.

The terms “nucleic acid”, “nucleotide sequence”, and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms include known analogs of natural nucleotides and nucleotides that are modified in the base, sugar and/or phosphate moieties. Analogs of a particular nucleotide have the same base-pairing specificity (e.g., an analog of A base pairs with T). A polynucleotide may include one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified by, for example, conjugation with a labeling component or target-binding component. A nucleotide sequence may incorporate non-nucleotide components. The terms also include nucleic acids including modified backbone residues or linkages, that (i) are synthetic, naturally occurring, and non-naturally occurring, and (ii) have similar binding properties as a reference polynucleotide (e.g., DNA or RNA). Examples of such analogs include phosphorothioates, phosphoramidates, methyl phosphonates, chiral-methyl phosphonates, 2-O-methyl ribonucleotides, peptide-nucleic acids (PNAs), and morpholino structures.

The term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

In particular embodiments, the term “gene” refers to a nucleotide sequence that encodes a protein (e.g., a variant Factor VIII), a negative selection marker, a selectable marker, or gRNA, as described herein. This definition includes various sequence polymorphisms, mutations, and/or sequence variants wherein such alterations do not substantially affect the function of the encoded protein or gRNA. The nucleic acid sequences can include both the full-length nucleic acid sequences as well as non-full-length sequences derived from a full-length protein. The sequences can also include degenerate codons of the native sequence or sequences that may be introduced to provide codon preference in a specific cell type. In particular embodiments, the term “gene” may include not only coding sequences but also regulatory regions such as promoters, enhancers, 5′ UTR, 3′UTR, termination regions, and non-coding regions. The term further can include all introns and other DNA sequences spliced from an mRNA transcript, along with variants resulting from alternative splice sites. Gene sequences encoding a molecule can be DNA or RNA that directs the expression of the molecule. These nucleotide sequences may be a DNA strand sequence that is transcribed into RNA or an RNA sequence that is translated into protein.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., gRNA, siRNA). In particular embodiments, a gene encodes or codes for a protein if transcription of DNA and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function. In particular embodiments, a gene encodes or codes for a functional polynucleotide when transcription of the gene produces the functional polynucleotide. In particular embodiments, the functional polynucleotide includes gRNA.

The terms “regulatory sequences”, “regulatory elements”, and “control elements” are interchangeable and refer to polynucleotide sequences that are upstream (5′ non-coding sequences), within, or downstream (3′ non-translated sequences) of a polynucleotide sequence to be transcribed or expressed. In particular embodiments, upstream and downstream relate to the 5′ to 3′ direction, respectively, in which RNA transcription takes place. In particular embodiments, upstream is toward the 5′ end of a nucleic acid and downstream is toward the 3′ end of a nucleic acid. Regulatory sequences influence, for example, the timing of transcription, amount or level of transcription, RNA processing or stability, and/or translation of a polynucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

The term “operably linked” refers to polynucleotide sequences or amino acid sequences placed into a functional relationship with one another. For instance, a promoter or enhancer is operably linked to a coding sequence or to a non-coding sequence (e.g., gRNA) if it regulates, or contributes to the modulation of, the transcription of the coding or non-coding sequence. In particular embodiments, regulatory sequences operably linked to a coding sequence or non-coding sequence are typically contiguous to the coding sequence or non-coding sequence. However, enhancers can function when separated from a promoter by up to several kilobases or more. Accordingly, some polynucleotide elements may be operably linked but not contiguous.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include the AFP (α-fetoprotein) promoter, amylase 1C promoter, aquaporin-5 (AP5) promoter, al-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, CMV (cytomegalovirus viral) promoter, minCMV promoter, COX-2 promoter, CXCR4 promoter, desmin promoter, E2F-1 promoter, EF1α (elongation factor lα) promoter, EGR1 promoter, elF4A1 promoter, elastase-1 promoter, endoglin promoter, FerH promoter, FerL promoter, fibronectin promoter, Flk-1 promoter, Flt-1 promoter, GAPDH promoter, GFAP promoter, Gp1bα promoter, GPIIb promoter, GRP78 promoter, GRP94 promoter, HE4 promoter, hGR1/1 promoter, hNIS promoter, Hsp68 promoter, Hsp68 minimal promoter, HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, Nphsl promoter, OG-2 promoter, PGK (Phospho Glycerate kinase) promoters, PGK-1 promoter, polymerase III (Pol III) promoter, PSA promoter, ROSA promoter, Rous Sarcoma Virus (RSV) long-terminal repeat (LTR) promoter, SP-B promoter, stabilin-2 promoter, Survivin promoter, SV40 (simian virus 40) promoter, SYN1 promoter, SYT8 gene promoter, Tie2 promoter, TRP1 promoter, Tyr promoter, ubiquitin B promoter, VE-cadherin promoter, and WASP promoter. In particular embodiments, the promoter includes ICAM2 promoter, stabilin-2 promoter, Tie2 promoter, Flk-1 promoter, or VE-cadherin promoter. In particular embodiments, the promoter is a megakaryocyte specific promoter, Gp1bα promoter. In particular embodiments, the promoter includes an LSEC-specific promoter, a hepatocyte specific promoter, or a ubiquitous promoter. In particular embodiments, an LSEC-specific promoter includes an ICAM2 promoter, Stabukum-2m promoter, Tie2 promoter, Flk-1 promoter, or VE-cadherin promoter. In particular embodiments, the hepatocyte-specific promoter includes a human α1-antitrypsin (hAAT) promoter. In particular embodiments, the ubiquitous promoter includes an SV40 promoter, a CMV promoter, a PGK promoter, or a CAG promoter.

In particular embodiments, an “enhancer” or an “enhancer element” is a cis-acting sequence that increases the level of transcription associated with a promoter, and can function in either orientation relative to the promoter and the coding sequence that is to be transcribed, and can be located upstream or downstream relative to the promoter or the coding sequence to be transcribed. There are art-recognized methods and techniques for measuring function(s) of enhancer element sequences. A particular example of an enhancer includes the ubiquitous chromatin opening element (UCOE). In particular embodiments, the enhancer includes a hepatic control region (HCR).

Nuclear localization signals (NLS) are generally short peptides that act as a signal fragment that mediates the transport of proteins from the cytoplasm into the nucleus. Examples of nuclear localization signals are known in the art (see, e.g., Lange et al., J. Biol. Chem., 2007, 282:5101-5105). Particular examples include the NLS from SV40 (PKKKRKV; SEQ ID NO: 19) or the NLS from nucleoplasmin (RPAATKKAGQAKKK; SEQ ID NO: 20).

MicroRNAs (miRNAs) are small endogenous non-coding RNAs of 22 nt in length that take crucial roles in many biological processes. These short RNAs regulate the expression of mRNAs by binding to their 3′-UTRs or by translational repression. miRNAs typically affect the extent to which specific mRNAs are translated into proteins by enhancing degradation rates of the mRNAs to which they bind. The selectivity of which mRNAs are degraded is due to qualitative and concentration differences among the miRNAs produced by each cell type, and on the miRNA binding affinities for the slightly differing miR target site (miRTS) sequences. By inserting different numbers of miRTs with high-to-low binding affinities into the non-coding regions of any desired product's cDNA, it is thus possible to adjust product levels in specific tissue or cell subtypes with even greater precision than can be achieved via transcriptional controls alone. Finer tuning of product levels between tissue or cell types can thus be obtained by selecting appropriate miRTSs based on the desired tissue or cellular expression. In particular embodiments, a miRNA target site (also referred to as miRNA target sequence) is incorporated into the 3′UTR. In particular embodiments, the miRNA target sequence includes miRT-122 and/or miRT-142-3p. In particular embodiments, miRT-122 inhibits expression in hepatocytes. In particular embodiments, miRT-142-3p inhibits expression in hematopoietic cells.

In particular embodiments, regulatory elements include an enhancer, a promoter, a FVIII coding sequence, and a 3′UTR. In particular embodiments, the 3′UTR can include an miRNA target sequence. In particular embodiments, the genetic construct can further include a nuclear localization signal.

As described more fully below, genetic constructs disclosed herein can also include one or more sequences to facilitate targeted genetic engineering, such as homology arms.

(iii) Vectors. In particular embodiments, a gene encoding a variant Factor VIII can be introduced into cells in a vector. A “vector” is a nucleic acid molecule that is capable of transporting another nucleic acid (e.g., genetic construct). Vectors may be, e.g., plasmids, cosmids, viruses, or phage. In particular embodiments, a vector carries the genetic construct into a cell.

Vectors derived from viruses can be used for gene delivery. Viruses that can be used include adenoviruses, adeno-associated viruses (AAV), and alphaviruses. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19:673-686.

In particular embodiments, vectors that can be used include retroviral vectors (see Miller, et al., 1993, Meth. Enzymol. 217:581-599). The term “retroviral vector” refers to a viral vector or plasmid containing structural and functional genetic elements, or portions thereof, that are primarily derived from a retrovirus. In particular embodiments, the transfer results in integration of the nucleic acid into the genome of the cell.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Where reference is made herein to elements such as cloning sites, promoters, regulatory elements, heterologous nucleic acids, etc., it is to be understood that the sequences of these elements are present in RNA form in the retroviral particles and are present in DNA form in the DNA plasmids.

In particular embodiments, a retroviral vector can include a lentiviral vector. The term “lentiviral vector” refers to a viral vector or plasmid containing structural and functional genetic elements, or portions thereof, that are primarily derived from a lentivirus. “Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV). A variety of lentiviral vectors are known in the art (Naldini et al. (1996) Science 272(5259): 263-267; Naldini et al. (1996) Proceedings of the National Academy of Sciences 93(21): 11382-11388; Zufferey et al. (1997) Nature biotechnology 15(9): 871-875; Dull et al. (1998) Journal of virology 72(11): 8463-8471; U.S. Pat. Nos. 6,013,516; and 5,994,136), many of which may be adapted to produce a viral vector or transfer plasmid.

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Therapeutically effective amounts of vectors within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(iv) Targeted Genetic Engineering. Within the teachings of the current disclosure, any gene editing system capable of precise sequence targeting and modification can be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. Alternatively, a targeting element and a cutting element can be linked together into one dual purpose molecule. Different gene editing systems can adopt different components and configurations while maintaining the ability to precisely target, cut, and modify selected genomic sites.

Engineered guide RNA associated with nucleases which target specific DNA sequences predictably generate DNA double strand breaks (DSB) at the targeted sequence. Use of gene editing systems to induce DSB can provide promising therapies when removal or silencing of a problematic gene (e.g., generating a loss-of-function mutation or creating an indel mutation or repair) is needed. Thus, gene-editing systems can be engineered to create a DSB at a desired target in a genome of a cell and harness the cell's endogenous mechanisms to repair the induced break by non-homologous end joining (NHEJ).

When insertion of a therapeutic nucleic acid sequence is intended, the systems can also include a homology-directed repair template (also referred to herein as a DNA repair template) which can include homology arms associated with the therapeutic nucleic acid sequence. In this instance, engineered guide RNA is again associated with nucleases which target specific DNA sequences predictably generating DSB at the targeted sequence. Following creation of a DSB at the desired target in the genome of a cell, the cell's endogenous mechanisms to repair the induced break is harnessed by homology repair, such as HDR) homology-mediated end joining (HMEJ), homology-independent targeted integration (HITI)-associated microhomology-mediated end joining (MMEJ), or HITI-associated non-homologous end joining (HITI-NHEJ) generally depending on the length of homology arms (e.g., as used herein HDR occurs if a region of homology is >75 bp and HITI occurs if a region of homology is <75 bp).

For gene addition or correction, homology-directed repair (HDR) of a DSB can be used. In this situation, gene-editing components generally include the engineered guide RNA and nuclease, and a homology-directed repair template with homology to the target DSB locus flanking a therapeutic gene.

HDR refers to DNA repair that takes place in cells, for example, during repair of double-stranded and single-stranded breaks in DNA. HDR requires nucleotide sequence homology between sequences of an HDR template and the target nucleic acid to repair the sequence where the break occurred in the target nucleic acid. In particular embodiments, the HDR template includes a non-homologous donor polynucleotide (donor sequence) flanked by two regions of homology (i.e., the homology arms), such that HDR between the target nucleic acid region and the two flanking homology arms results in insertion of the non-homologous donor polynucleotide at the target region. In particular embodiments, the homology arms will have at least 50% sequence identity to a genomic sequence with which recombination is desired. In particular embodiments, at least 60%, at least 70%, at least 80%, at least 90%, at least 95%, at least 98%, at least 99%, or at least 99.9% sequence identity is present between a homology arm and a target nucleic acid sequence. In particular embodiments, each homology arm can be 50 base pairs (bp), 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 475 bp, 500 bp, 525 bp, 550 bp, 575 bp, 600 bp, 625 bp, 650 bp, 675 bp, 700 bp, 725 bp, 750 bp, 775 bp, 800 bp, 825 bp, 850 bp, 875 bp, 900 bp, 925 bp, 950 bp, 975 bp, 1000 bp, 1250 pb, 1500 bp, or longer. In particular embodiments, the length of each homology arm can depend on the size of the donor polynucleotide and the target nucleic acid.

A DNA repair template includes a polynucleotide that can be directed to and inserted into a target site of interest to modify a target nucleic acid (e.g., in a genome). In particular embodiments, a DNA repair template is used as a template to copy the donor polynucleotide sequences into the target site of interest. Repair of the break in the target nucleic acid sequence can result in the transfer of genetic information (i.e., polynucleotide sequences) from the DNA repair template at the site or in close proximity of the break in the target nucleic acid sequence. Accordingly, new genetic information (i.e., polynucleotide sequences) may be inserted or copied at a target nucleic acid site. HDR may result in alteration of the target nucleic acid sequence (e.g., insertion, deletion, mutation) if the DNA repair template sequence differs from the target nucleic acid sequence. The DNA repair template may contain at least one or more single base changes, insertions, deletions, inversions or rearrangements with respect to the genomic sequence, so long as sufficient homology is present between sequences of the homology arms and the target nucleic acid sequence to support HDR. In particular embodiments, an entire DNA repair template, a portion of the DNA repair template, or a copy of the donor polynucleotide is integrated at the site of the target nucleic acid sequence. In particular embodiments, insertion or copying of the DNA repair template leads to correction of endogenous genes (e.g., Factor VIII genes).

In particular embodiments, HMEJ-based repair is used to increase precision gene editing in non-dividing cells. The DNA template in HMEJ is similar to HDR, but the homologous regions are flanked by sgRNA targeting sites. Compared to MMEJ, HMEJ harbors longer homology arms to achieve higher gene repairing efficiency. In particular embodiments, the DNA repair template is excised from a plasmid or AAV vector.

In particular embodiments, the donor polynucleotide can include a gene of interest. In particular embodiments, a gene of interest includes a polynucleotide that encodes a variant Factor VIII. In particular embodiments, the gene of interest can include a polynucleotide sequence to modify a regulatory sequence of a gene, to introduce a regulatory sequence to a gene (e.g., a promoter, an enhancer, an internal ribosome entry sequence, a start codon, a stop codon, a localization signal, or polyadenylation signal), or to modify a nucleic acid sequence (e.g., introduce a mutation). Gene sequences encoding variant Factor VIII can be readily identified by those of ordinary skill in the art.

Particular embodiments use the CRISPR gene editing system to provide functional variant Factor VIII expression.

Particular embodiments combine CRISPR RNA (crRNA) and trans-activating CRISPR RNA (tracrRNA) into a guide RNA (gRNA) or synthetic single guide RNA (sgRNA). In particular embodiments, a gRNA or sgRNA are the RNA molecules used to specify a particular target area for cleavage by a nuclease. In particular embodiments, gRNA includes two parts: crRNA, a nucleotide sequence (e.g., 17-20 nucleotides) complementary to the target DNA, and a tracrRNA sequence, which serves as a binding scaffold for the Cas nuclease. When the crRNA and tracrRNA elements are combined into a single RNA molecule, the molecule is referred to as sgRNA, though gRNA and sgRNA are often used interchangeably. In particular embodiments, gRNA includes sgRNA. For certain gene editing systems, the target sequence may be adjacent to a PAM (e.g., 5′-20 nt target-NGG-3′) or can include a PAM. In particular embodiments, guide RNA (gRNA) includes a target site adjacent to the PAM targeted by the genome editing complex. The gRNA can include at least the 16, 17, 18, 19, 20, 21, or 22 nucleotides adjacent to the PAM.

Exemplary CRISPR-Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

A single Cas enzyme can be programmed by a gRNA molecule to site-specifically cleave a specific target nucleic acid. Cas9 is an exemplary Type II CRISPR Cas protein. Cas9 includes two distinct endonuclease domains (HNH and RuvC/RNase H-like domains), one for each strand of the target nucleic acid. RuvC and HNH together produce DSBs; separately each domain can produce single-stranded breaks. Base-pairing between the gRNA and target nucleic acid causes DSBs due to the endonuclease activity of Cas9. Binding specificity is determined by both gRNA-target nucleic acid base pairing and the PAM juxtaposed to the DNA complementary region. In particular embodiments, the CRISPR system only requires a minimal set of two molecules—the Cas protein and the gRNA.

A large number of Cas9 orthologs are known in the art (Fonfara et al. Nucleic Acids Research (2014) 42:2577-2590; Chylinski et al. Nucleic Acids Research (2014) 42:6091-6105; Esvelt et al. Nature Methods (2013) 10:1116-1121). A number of orthogonal Cas9 proteins have been identified including Cas9 proteins from Neisseria meningitidis, Streptococcus thermophilus and Staphylococcus aureus. Other Class 2 Cas proteins that can be used include Cas12a (Cpf1), Cas13a (C2c2), and Cas13B (C2c6).

In particular embodiments, polynucleotide sequences encoding mutant forms of Cas9 nuclease can be used in genetic constructs of the disclosure. For example, a Sniper Cas9, a variant of Cas9 with optimized specificity (minimal off-target effects) and retained on-target activity can be used (Lee et al. J Vis Exp. 2019 Feb. 26; (144); Lee et al. Nat Commun. 2018 Aug. 6;9(1): 3048; WO 2017/217768). As another example, a mutant Cas9 nuclease containing a D10A amino acid substitution can be used. This mutant Cas9 has lost double-stranded nuclease activity present in the wild type Cas9 but retains partial function as a single-stranded nickase. This mutant Cas9 generates a break in the complementary strand of DNA rather than both strands. This allows repair of the DNA template using a high-fidelity pathway rather than non-homologous end joining (NHEJ). The higher fidelity pathway prevents formation of insertions/deletions at the targeted locus while maintaining ability to undergo homologous recombination (Cong et al. Science (2013) 339(6121): 819-823). Paired nicking has been shown to reduce off-target activity by 50- to 1,500-fold in cell lines (Ran et al. Cell (2013) 154(6): 1380-1389).

In particular embodiments, a Cas protein can be fused to a heterologous polypeptide that provides for subcellular localization. Such heterologous peptides include, for example, a nuclear localization signal (NLS) such as the SV40 NLS for targeting to the nucleus (e.g., Lange et al. (2007) J. Biol. Chem. 282:5101-5105). Such subcellular localization signals can be located at the N-terminus, the C-terminus, or anywhere within the Cas protein. An NLS can include a stretch of basic amino acids and can be a monopartite sequence or a bipartite sequence.

In particular embodiments, a Cas protein can also include a heterologous polypeptide for ease of tracking or purification, such as a fluorescent protein, a purification tag, or an epitope tag. Examples of tags include green fluorescent protein (GFP), glutathione-S-transferase (GST), myc, Flag, hemagglutinin (HA), Nus, Softag 1, Softag 3, Strep, polyhistidine, biotin carboxyl carrier protein (BCCP), maltose binding protein (MBP), and calmodulin.

The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, increasing the efficiency of HDR.

Additional information regarding CRISPR-Cas systems and components thereof are described in U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233, 8,999,641, and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473, WO2015/089486, WO2016/205711, WO2017/106657, WO2017/127807, and applications related thereto.

Teachings of the disclosure in relation to CRISPR can be applied to other gene editing systems that similarly utilize nucleases.

Particular embodiments utilize zinc finger nucleases (ZFNs) as gene editing agents. For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933, 113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140 as well as Gaj et al., Nat Methods, 2012, 9(8): 805-7; Ramirez et al., Nucl Acids Res, 2012, 40(12): 5560-8; Kim et al., Genome Res, 2012, 22(7): 1327-33; Urnov et al., Nature Reviews Genetics, 2010, 11:636-646; Miller, et al. Nature biotechnology 25, 778-785(2007); Bibikova, et al. Science 300, 764(2003); Bibikova, et al. Genetics 161, 1169-1175(2002); Wolfe, et al. Annual review of biophysics and biomolecular structure 29, 183-212(2000); Kim, et al. Proceedings of the National Academy of Sciences of the United States of America 93, 1156-1160(1996); and Miller, et al. The EMBO journal 4, 1609-1614(1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12^{th} and 13^{th} positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

The present disclosure can utilize base editing systems, for example those that utilize a deaminase. Deamination of a nucleotide can cause changes in the sequence of a nucleic acid. Deamination of adenosine (A) results in an A-T to G-C transition. Deamination of cytosine (C) results in a C-G to T-A transition. Collectively, cytosine and adenosine deamination can be used to cause transitions from A to G, T to C, C to T, or G to A.

Examples of cytosine deaminase enzymes (CBEs) include APOBEC1, APOBEC3A, APOBEC3G, evoAPOBEC, BE4-YE1, CDA1, and AID. APOBEC1. Examples of adenosine base editors (ABEs) include a mutant TadA adenosine deaminases (TadA*) that accepts DNA as its substrate.

Particular base editing systems include a deaminase associated with a DNA binding domain such as a catalytically impaired nuclease domain. The DNA binding domain can localize the deaminase to a target nucleic acid in which one or more nucleotides are deaminated by the deaminase. Catalytically impaired nuclease domains are engineered from reference nuclease domain sequences but have a reduced or no ability to cause DSBs as compared to the reference (e.g., a wild-type) sequence.

Base editing systems can include a DNA glycosylase inhibitor that serves to override natural DNA repair mechanisms that might otherwise repair the intended base editing. A DNA glycosylase inhibitor can be a uracil DNA glycosylase inhibitor protein (UGI). One exemplary UGI is described in Wang et al. (Gene 99:31-37, 1991).

Exemplary base editing enzymes are described in e.g., Komor 2016 Nature 533:420-424; Rees 2017 Nat. Commun. 8:15790), Koblan 2018 Nat. Biotechnol 36(9): 843-846; Komor 2017 Sci. Adv. 3(8): eaao4774), Kim 2017 Nat. Biotechnol. 35:475-480), Li 2018 Nat. Biotechnol. 36:324-327)), Nishida 2016 Science 353(6305): aaf8729)), Nishimasu 2018 Science 361(6408): 1259-1262)), Hu 2018 Nature 556:57-63)), Gehrke 2018 Nat. Biotechnol. 36(10): 977-982)), Wang 2018 Nat. Biotechnol. 36:946-949)), Jiang 2018 Cell Res. 28(8): 855-861)), Rees 2018 Nat. Rev Genet. 19(12): 770-788 and Kantor 2020 Int. J. Mol. Sci. 21(17): 6240.

Dual base editors can edit both adenine and cytosine. (see, e.g., Sakata 2020 Nature Biotechnology, 38(7), 865-869; Grünewald 2020 Nat. Biotechnol. 38:861-864), and Zhang 2020 Nat. Biotechnol. 38:856-860).

In certain examples, a genetic construct of the disclosure includes elements to transcribe gRNA, express nuclease protein, and provide for expression of variant Factor VIII.

When targeted genetic engineering approaches are utilized, genes can be inserted at any location suitable for expression of the genetic construct. In particular embodiments, the genetic construct can replace the native factor VIII gene. In particular embodiments, the genetic construct can be inserted into any other suitable location within the genome for expression of the Factor VIII variant. In particular embodiments, the genetic construct can be inserted within a genomic safe harbor or a landing pad. Genomic safe harbor sites are intragenic or extragenic regions of the genome that are able to accommodate the predictable expression of newly integrated DNA without adverse effects on the host cell. A useful safe harbor must permit sufficient transgene expression to yield desired levels of the encoded molecule. A genomic safe harbor site also must not alter cellular functions. Methods for identifying genomic safe harbor sites are described in Sadelain et al., Nature Reviews (2012); 12:51-58; and Papapetrou et al., Nat Biotechnol. (2011) January; 29(1): 73-8. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, a genomic safe harbor meets criteria described herein and also demonstrates a 1:1 ratio of forward: reverse orientations of lentiviral integration further demonstrating the loci does not impact surrounding genetic material.

Particular genomic safe harbors sites include CCR5, HPRT, AAVS1, Rosa and albumin. See also, e.g., U.S. Pat. Nos. 7,951,925 and 8,110,379; U.S. Publication Nos. 20080159996; 201000218264; 20120017290; 20110265198; 20130137104; 20130122591; 20130177983 and 20130177960 for additional information and options for appropriate genomic safe harbor integration sites.

A landing pad is a synthetic segment of DNA sequence that has no specific function by itself but that has been designed to accelerate and secure the genomic integration of one or multiple heterologous genes optimizing their expression and stability.

(v) Nanoparticles and Microbubbles. Nanoparticle types include liposomes (microscopic vesicles including at least one concentric lipid bilayer surrounding an aqueous core), liposomal nanoparticles (a liposome structure used to encapsulate another smaller nanoparticle within its core), and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum, and silica).

In particular embodiments, ultrasound-mediated gene delivery (UMGD) is used for delivery of the genetic construct described herein. Effective UMGD relies on the presence of microbubbles, which have been demonstrated to significantly enhance gene transfer efficiency.

Composition and methods for forming microbubbles as ultrasound contrast agents are well established in the art. Therefore, the person skilled in the art knows the materials and methods to form the microbubbles used in the present disclosure. See, e.g., Ultrasound Contrast Agents: Basic Principles and Clinical Applications by B. B. Goldberg, et al. (Eds.), Taylor & Francis (2nd Edition, 2001). Examples of procedures for the preparation of microbubbles are described in: U.S. Pat. Nos. 4,446,442, 4,684,479, 4,718,433, 5,088,499, 5,123,414, 5,271,928, 5,413,774, 5,445,813, 5,556,610, 5,597,549, 5,686,060, 5,773,527, 5,798,091, 5,827,504, 6,217,850, 6,416,740, 6,443,898, and European Patent 0458745.

Microbubbles include a shell surrounding an internal void including a gas. Because of the surface energy involved in formation of the interface between the different phases, the microbubbles are expected to be relatively spherical in shape, as a result of minimization of the area of the interface.

In particular embodiments, microbubbles have a diameter between 0.2 and 300 μm. In particular embodiments, microbubbles have a diameter no more than 200, 100, 50, 10, 8, 7, 6, or 5 μm (measured as average number weighted diameter of the microbubble composition). In particular embodiments, microbubbles have a diameter in a range of 0.2-3 μm. In particular embodiments, microbubbles have an average diameter of 1 μm.

The microbubble shell typically includes a surfactant or a polymer. Surfactants suitable for use in microbubble preparation include any compound or composition that aids in the formation and maintenance of a microbubble by forming a layer at the interface between the gas and the medium, usually an aqueous medium, containing the microbubble. The surfactant may include a single compound or a combination of compounds. It will be appreciated by the person skilled in the art that a wide range of compounds capable of facilitating formation of the microbubbles can be used in the present disclosure.

In particular embodiments, the microbubbles are prepared according to methods described in Sun et al., (J Control Release, 182:1111-120, 2014) and U.S. application Ser. No. 17/051,141. In particular embodiments, microbubble shells are composed of lipids at a 82:10:8 molar ratio of 1,2-distearoyl-sn-glycero-3-phosphocholine (DSPC), 1,2-distearoyl-sn-glycero-3-phosphate (DSPA), and N-(Carbonylmethoxypolyethyleneglycol 5000)-1,2-distearoyl-sn-glycero-e-phospho-ethanolamine (MPEG-5000-DSPE).

Therapeutically effective amounts of nanoparticles and/or microbubbles within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(vi) Compositions for Administration. Recombinant proteins of Factor VIII variants described herein, gene-editing components (e.g., genetic construct, sgRNA, nuclease, DNA repair templates, vectors), or gene-editing components incorporated within nanoparticles (all collectively “active ingredients”) can be formulated alone or in combination into compositions for administration to subjects. Salts and/or pro-drugs of active ingredients can also be used.

Exemplary generally used pharmaceutically acceptable carriers include any and all absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredient or helps to prevent denaturation or adherence to the container wall. Typical stabilizers can include polyhydric sugar alcohols; amino acids; organic sugars or sugar alcohols; sulfur-containing reducing agents; proteins such as human serum albumin, bovine serum albumin, gelatin or immunoglobulins; hydrophilic polymers such as polyvinylpyrrolidone; monosaccharides such as xylose, mannose, fructose and glucose; disaccharides; trisaccharides, and polysaccharides.

The compositions disclosed herein can be formulated for administration by, for example, injection. For injection, compositions can be formulated as aqueous solutions, such as in buffers including Hanks' solution, Ringer's solution, or physiological saline, or in culture media, such as Iscove's Modified Dulbecco's Medium (IMDM). Injectable compositions can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of active ingredients and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Compositions disclosed herein can be formulated for administration by, for example, injection, infusion, perfusion, or lavage. The compositions disclosed herein can further be formulated for intraosseous, intravenous, intradermal, intraarterial, intranodal, intralymphatic, intraperitoneal, intralesional, intraprostatic, intravaginal, intrarectal, intrathecal, intramuscular, intravesicular, and/or subcutaneous administration and more particularly by intraosseous intravenous, intradermal, intraperitoneal, intramuscular, and/or subcutaneous injection.

In particular embodiments, compositions disclosed herein can be formulated for administration by portal vein injection.

(vii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions (e.g., recombinant proteins, nucleic acids, and/or nanoparticles) disclosed herein. Treating subjects includes delivering therapeutically effective amounts. Therapeutically effective amounts include those that provide effective amounts and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of hemophilia A or in a clinical trial assessing the efficacy and safety of a hemophilia treatment.

A “therapeutic treatment” includes a treatment administered to a subject who displays symptoms or signs of hemophilia A and is administered to the subject for the purpose of diminishing or eliminating those signs or symptoms of hemophilia A. The therapeutic treatment can reduce, control, or eliminate the effects of hemophilia A.

Functions as an effective amount or therapeutic treatment are not mutually exclusive, and in particular embodiments, administered dosages may accomplish more than one treatment type.

In particular embodiments, therapeutically effective amounts provide reduction in symptoms of hemophilia A. A reduction in symptoms of hemophilia A can include an increase in functional Factor VIII expression and improved blood clotting following damage to a blood vessel.

In particular embodiments, the administration of a therapeutically effective amount results in an increase of functional Factor VIII in a subject's plasma of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100% compared to the level of functional Factor VIII in the subject's plasma prior to the administration.

In particular embodiments, the administration of a therapeutically effective amount results in a decrease in bleeding by a subject following injury to a blood vessel of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100% than the level observed prior to the administration following comparable damage to a blood vessel.

In certain examples, therapeutically effective amounts are confirmed by measuring and detecting an improvement in activated partial thromboplastin time (aPTT), complete blood count (CBC), or fibrinogen test.

Methods disclosed herein provide reduced immune responses against therapeutic forms of Factor VIII. Reduced immune responses can be reduced antibody responses (e.g., reduced IgG antibody responses). In addition to reducing the immune response, methods disclosed herein provide Factor VIII variants with increased expression, secretion, stability, and FVIII functional activity. In particular embodiments, FVIII expression can be measured using Western blotting, enzyme-linked immunoassay (ELISA), fluorescence-based assays, or other immunoassays. In particular embodiments, FVIII secretion can be measured using a one-stage clotting assay, FVIII-specific ELISA, or other assays used to measure expression. In particular embodiments, FVIII stability can be measured using fluorescence-based activity assays, circular dichroism (CD) spectroscopy, mass spectrometry, bleach-chase method, cycloheximide-chase method, pulse-chase method, or differential scanning calorimetry (DSC). In particular embodiments, FVIII functional activity can be measured using aPTT, CBC, a fibrinogen test, a thrombin generation assay (TGA), rotational thromboelastometry assay, ferric chloride (FeCl₃)-induced thrombosis injury model, a tail clip assay, or measuring blood flow rate.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of hemophilia A, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, for example, injection, inhalation, infusion, perfusion, or lavage. Routes of administration can include intraosseous, intravenous, intradermal, intraparenteral, intranasal, intramuscular, and/or subcutaneous.

In particular embodiments, compositions disclosed herein can be administered by portal vein injection.

In particular embodiments, compositions disclosed herein can be administered by ultrasound. Ultrasound is high frequency sound, with a frequency of 10 kHz or greater. In particular embodiments, the ultrasound frequency ranges from 20 kHz to 20 MHz. In particular embodiments, the ultrasound frequency range is in frequencies used in diagnostic sonography scanners, which are in the range from 1 MHz to 15 MHz. The frequency and intensity of ultrasound used is determined by the requirement to achieve selective microbubble destruction at the site of delivery. The requisite parameters for optimizing microbubble destruction have been studied, and are known to the person skilled in the art. See, e.g., U.S. application Ser. No. 17/051,141 and K. W. Walker, et al., (Invest. Radiol., 1997, 32(12), 728-34). In particular embodiments, compositions disclosed here can be administered by a pulsed therapeutic ultrasound transducer applied to the surface of the liver for 10 seconds to 10 minutes at 2-18 MHz frequency and 1-20 Hz pulse repetition frequency (PRF). In particular embodiments, compositions disclosed here can be administered by a pulsed therapeutic ultrasound transducer applied to the surface of the liver for one minute at 1.1 MHz frequency and 14 Hz PRF. In particular embodiments, the ultrasound can be applied at low energy (e.g., 50 W/cm², 150 us PD) or high energy (e.g., 110 W/cm², 150 us PD). In particular embodiments, low energy targets endothelial cells. In particular embodiments, high energy targets hepatocytes. In particular embodiments, low energy ultrasound includes an intensity ranging from 0-75 W/cm². In particular embodiments, high energy ultrasound includes an intensity greater than 76 W/cm². In particular embodiments, ultrasound is administered transcutaneously. In particular embodiments, diagnostic ultrasound is utilized to guide the administration of therapeutic transcutaneous ultrasound.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

(viii) Exemplary Embodiments.
1. A factor VIII protein including a deletion of glycans at residue 2118 within the C1 domain.
2. A factor VIII protein including a mutation at residue 2118, wherein the mutation reduces or eliminates glycosylation at residue 2118.
3. The factor VIII protein of embodiment 2, wherein the mutation includes an asparagine to glutamine mutation.
4. The factor VIII protein of embodiments 1 or 2, further including a B domain deletion (BDD-FVIII) or a B domain truncation.
5. The factor VIII protein of embodiment 4, wherein the B domain truncation includes a truncated B domain having 226 amino acids and only 6 N-linked glycosylation sites (N6) or a truncated B domain having 17 amino acids (V3).
6. The factor VIII protein of any of embodiments 1-5, further including a mutation in the B domain at residue 1645.
7. The factor VIII protein of embodiment 6, wherein the mutation in the B domain includes an arginine to histidine mutation.
8. The factor VIII protein of any of embodiments 1-7, further including a mutation in the A1 domain.
9. The factor VIII protein of embodiment 8, including V861, F105Y, S108A, E115D, H117Q, L129F, K132G, Q134H, T147M, and P152L mutations in the A1 domain (X10).
10. The factor VIII protein of embodiments 8 or 9, wherein the mutation in the A1 domain includes F309S.
11. The factor VIII protein of any of embodiments 1-10, further including a furin-cleavage site deletion.
12. The factor VIII protein of any of embodiments 1-11, further including mutations in the C1 and C2 domains.
13. The factor VIII protein of embodiment 12, including V18571, H1859R, M1907K, M1926K, L1975V, A1993V, H2007Q, D2066E, K2085M, Q2113H, S2157N, and R2159H mutations (K12).
14. The factor VIII protein of any of embodiments 1-13, wherein the factor VIII protein includes a BDD-FVIII-N2118Q, BDD-FVIII*-N2118Q, F8/N6-N2118Q, F8/V3-N2118Q, BDD-FVIII-RH-N2118Q, BDD-FVIII*-RH-N2118Q, F8/N6RH-N2118Q, F8/V3RH-N2118Q, BDD-FVIII-X10-N2118Q, F8/N6-X10-N2118Q, BDD-FVIII*-X10-N2118Q, F8/V3×10-N2118Q, BDD-FVIII-X10-RH-N2118Q, BDD-FVIII*-X10-RH-N2118Q, F8/N6-X10-RH-N2118Q, F8/V3-X10-RH-N2118Q, BDD-FVIII-F309S-N2118Q, BDD-FVIII*-F309S-N2118Q, F8/N6F309S-N2118Q, F8/V3F309S-N2118Q, BDD-FVIII-F309S-N2118Q-RH, BDD-FVIII*-F309S-N2118Q-RH, F8/N6-F309S-N2118Q-RH, F8/V3-F309S-N2118Q-RH, BDD-FVIII-K12-N2118Q, BDD-FVIII*-K12-N2118Q, F8/N6K12-N2118Q, F8/V3K12-N2118Q, BDD-FVIII-K12-N2118Q-RH, BDD-FVIII*-K12-N2118Q-RH, F8/N6-K12-N2118Q-RH, F8/V3-K12-N2118Q-RH, BDD-FVIII-X10-F309S-N2118Q, BDD-FVIII*-X10-F309S-N2118Q, F8/N6X10-F309S-N2118Q, F8/V3X10-F309S-N2118Q, BDD-FVIII-X10-F309S-N2118Q-RH, BDD-FVIII*-X10-F309S-N2118Q-RH, F8/N6-X10-F309S-N2118Q-RH, F8/V3-X10-F309S-N2118Q-RH, BDD-FVIII-X10-K12-N2118Q, BDD-FVIII*-X10-K12-N2118Q, F8/N6X10-K12-N2118Q, F8/V3X10-K12-N2118Q, BDD-FVIII-X10-K12-N2118Q-RH, BDD-FVIII*-X10-K12-N2118Q-RH, F8/N6-X10-K12-N2118Q-RH, F8/V3-X10-K12-N2118Q-RH, BDD-FVIII-K12-F309S-N2118Q, BDD-FVIII*-K12-F309S-N2118Q, F8/N6K12-F309S-N2118Q, F8/V3K12-F309S-N2118Q, BDD-FVIII-K12-F309S-N2118Q-RH, BDD-FVIII*-K12-F309S-N2118Q-RH, F8/N6-K12-F309S-N2118Q-RH, F8/V3-K12-F309S-N2118Q-RH, BDD-FVIII-X10-K12-F309S-N2118Q, BDD-FVIII*-X10-K12-F309S-N2118Q, F8/N6X10-K12-F309S-N2118Q, F8/V3X10-K12-F309S-N2118Q, BDD-FVIII-X10-K12-F309S-N2118Q-RH, BDD-FVIII*-X10-K12-F309S-N2118Q-RH, F8/N6-X10-K12-F309S-N2118Q-RH, or F8/V3-X10-K12-F309S-N2118Q-RH, wherein * indicates a furin cleavage site deletion.
15. The factor VIII protein of any of embodiments 1-14, wherein the factor VIII protein includes BDD-FVIII-N2118Q, BDD-FVIII-X10-N2118Q, BDD-F8/N6-N2118Q, or BDD-F8/N6-X10-N2118Q, or BDD-cF8-X10-N2118Q.
16. The factor VIII protein of embodiment 15, wherein the factor VIII protein is encoded by the sequence as set forth in SEQ ID NOs: 23, 27, 31, 34, or 35 or a sequence having at least 90% sequence identity to the sequence as set forth in SEQ ID NOs: 23, 27, 31, 34, or 35.
17. A nucleic acid encoding a factor VIII protein including a deletion of glycans at residue 2118 within the C1 domain.
18. A nucleic acid encoding a factor VIII protein of any of embodiments 1-16.
19. The nucleic acid of embodiment 18, having the sequence as set forth in SEQ ID NOs: 23, 27, 31, 34, or 35 or a sequence having at least 90% sequence identity to the sequence as set forth in SEQ ID NOs: 23, 27, 31, 34, or 35.
20. The nucleic acid of embodiments 18 or 19, having the sequence as set forth in SEQ ID NO: 21, 22, 24-26, 28-30, 32, or 33 with nucleotide substitutions to generate an N2118Q mutation.
21. The nucleic acid of embodiment 20, wherein the nucleotide substitutions include replacing AAT or AAC with CAA or CAG.
22. The nucleic acid of any of embodiments 18-21, within a genetic construct including a promoter operably linked to the nucleic acid.
23. The nucleic acid of embodiment 22, wherein the genetic construct further includes an enhancer operably linked to the nucleic acid.
24. The nucleic acid of embodiments 22 or 23, wherein the genetic construct further includes a 3′UTR.
25. The nucleic acid of embodiments 23 or 24, wherein the enhancer includes a ubiquitous chromatin opening element (UCOE) enhancer or a hepatic control region (HCR).
26. The nucleic acid of any of embodiments 22-25, wherein the promoter includes a liver sinusoidal endothelial cell (LSEC)-specific promoter.
27. The nucleic acid of embodiment 26, wherein the LSEC-specific promoter includes ICAM2, Stabilin-2, Tie2, Flk-1, or VE cadherin.
28. The nucleic acid of any of embodiments 22-25, wherein the promoter includes a hepatocyte-specific promoter.
29. The nucleic acid of embodiment 28, wherein the hepatocyte-specific promoter includes an hAAT promoter.
30. The nucleic acid of embodiment 22-25, wherein the promoter includes a ubiquitous promoter.
31. The nucleic acid of embodiment 30, wherein the ubiquitous promoter includes an SV40 promoter, CMV promoter, PGK promoter, or CAG promoter.
32. The nucleic acid of any of embodiments 24-31, wherein the 3′UTR includes an miRT-122 or an miRT-142-3p.
33. The nucleic acid of any of embodiments 22-32, wherein the genetic construct further includes a nuclear localization signal.
34. The nucleic acid of embodiment 33, wherein the nuclear localization signal includes an SV40 nuclear localization signal.
35. The nucleic acid of any of embodiments 18-34, within a vector for delivery to a cell.
36. The nucleic acid of embodiment 35, wherein the vector is a viral vector.
37. The nucleic acid of embodiment 36, wherein the viral vector is a lentiviral vector.
38. The nucleic acid of embodiment 36, wherein the viral vector is an adeno-associated viral vector (AAV).
39. The nucleic acid of any of embodiments 22-38, wherein the genetic construct further includes homology arms.
40. The nucleic acid of embodiment 39, wherein the homology arms are homologous to an endogenous factor VIII locus.
41. The nucleic acid of embodiment 39, wherein the homology arms are homologous to a site within a genomic safe harbor.
42. The nucleic acid of any of embodiments 18-41, wherein the nucleic acid includes cDNA.
43. A nanoparticle including the nucleic acid of any of embodiments 17-42.
44. A composition including (i) a factor VIII protein of any of embodiments 1-16, a nucleic acid of any of embodiments 17-41, and/or a nanoparticle of embodiment 43 and (ii) a pharmaceutically acceptable carrier.
45. A method of treating a subject for hemophilia, the method including administering a therapeutically effective amount of the composition of embodiment 44 to the subject, thereby treating the subject for the hemophilia.
46. The method of embodiment 45, wherein the hemophilia is hemophilia A.
47. The method of embodiments 45 or 46, wherein the administering includes intraosseous, intravenous, intradermal, intraarterial, intranodal, intralymphatic, intraperitoneal, intralesional, intraprostatic, intravaginal, intrarectal, intrathecal, intramuscular, intravesicular, and/or subcutaneous administration.
48. The method of embodiment 47, wherein the intravenous administration includes portal vein injection.
49. The method of any of embodiments 45-47, wherein the administering includes intraosseous administration.
50. The method of embodiments 45 or 47, wherein the administering utilizes ultrasound.
51. The method of any of embodiments 45-50, wherein the therapeutically effective amount has lowered immunogenicity as compared to native FVIII.
52. A method for expressing a genetic construct encoding a factor VIII protein within a population of cells, the method including administering the nucleic acid of any of embodiments 17-41 and/or the nanoparticle of embodiment 42 in a sufficient dosage and for a sufficient time to the population of cells thereby expressing the genetic construct within the population of cells.
53. The method of embodiment 52, wherein the population of cells is in vivo at the time of the administering.
54. The method of embodiment 52, wherein the cells are ex vivo at the time of the administering.
55. The method of any of embodiments 52-54, wherein the population of cells include liver sinusoidal endothelial cells (LSECs).
56. The method of any of embodiments 52-55, wherein the administering includes ultrasound-mediated gene delivery (UMGD).
57. The method of embodiment 56, wherein the UMGD utilizes a microbubble or a nanobubble.
58. The method of embodiment 57, wherein the microbubble has a diameter in a range of 0.2-3 microns (μm).
59. The method of embodiment 57, wherein the microbubble has an average diameter of 1 μm
60. The method of any of embodiments 57-59, wherein the microbubble or the nanobubble is delivered intravenously.
61. The method of any of embodiments 56-60, wherein the UMGD utilizes transcutaneous ultrasound.
62. The method of any of embodiments 56-61, wherein the UMGD utilizes a peak negative pressure in the range of 0.5-2.5 megapascals (MPa).
63. The method of any of embodiments 56-62, wherein the UMGD utilizes a pulse duration in the range of 18-2000 microseconds (μs).
64. The method of any of embodiments 56-63, wherein the UMGD utilizes a frequency in a range of 0.8-1.4 megahertz (MHz).
65. The method of any of embodiments 56-64, wherein the UMGD utilizes a frequency of 1.1 MHz.
66. The method of any of embodiments 56-65, wherein the UMGD utilizes a pulse repetition frequency (PRF) in a range of 1-50 Hertz (Hz).
67. The method of any of embodiments 56-65, wherein the UMGD utilizes a PRF of 14 Hz.
68. The method of any of embodiments 56-67, wherein the UMGD utilizes an intensity of 0-75 W/cm².
69. The method of any of embodiments 56-67, wherein the UMGD utilizes an intensity of 50 W/cm² and a pulse duration of 150 μs.
70. The method of any of embodiments 56-69, wherein the UMGD utilizes an intensity of 76-200 W/cm².
71. The method of any of embodiments 56-69, wherein the UMGD utilizes an intensity of 110 W/cm² and a pulse duration of 150 μs.
72. The method of any of embodiments 56-71, wherein the UMGD results in preferential delivery of the nucleic acid or nanoparticle to epithelial cells over hepatocytes.
73. The method of any of embodiments 52-72, wherein the administering includes intraosseous, intravenous, intradermal, intraarterial, intranodal, intralymphatic, intraperitoneal, intralesional, intraprostatic, intravaginal, intrarectal, intrathecal, intramuscular, intravesicular, and/or subcutaneous administration.
74. The method of embodiment 73, wherein the intravenous administration includes portal vein injection.
75. The method of any of embodiments 52-73, wherein the administering includes intraosseous administration.
76. The method of any of embodiments 52-75, wherein the subject is a human, mouse, canine, or non-human primate.
77. The method of any of embodiments 52-76, wherein the administering includes pipetting.

(ix) Experimental Examples. Example 1. Influence of N-glycosylation in the A and C domains on the immunogenicity of factor VIII.

Abstract. The most significant complication in hemophilia A treatment is the formation of inhibitors against factor VIII (FVIII) protein. Glycans and glycan-binding proteins are central to a properly functioning immune system. This example focuses on whether glycosylation of FVIII plays an important role in induction and regulation of anti-FVIII immune responses. The potential roles of four N-glycosylation sites including N41 and N239 in the A1 domain, N1810 in the A3 domain, and N2118 in the C1 domain of FVIII in moderating its immunogenicity were investigated. Glycomics analysis of plasma derived FVIII revealed that sites N41, N239, and N1810 contain mostly sialylated complex glycoforms, while high mannose glycans dominate at site N2118. A missense variant that substitutes Asparagine (N) to Glutamine (Q) was introduced to eliminate glycosylation on each of these sites. Following gene transfer of plasmids encoding BDD-FVIII and each of these four FVIII variants, it was found that specific activity of FVIII in plasma remained similar among all treatment groups. Slightly increased or comparable immune responses in N41Q, N239Q, and N1810Q FVIII variant plasmids treated mice and significantly decreased immune responses in N2118Q FVIII plasmid treated mice were observed when compared to BDD-FVIII plasmid treated mice. The reduction of inhibitor response by N2118Q FVIII variant was also demonstrated in AAV-mediated gene transfer experiments. Furthermore, a specific glycopeptide epitope surrounding the N2118 glycosylation site was identified and characterized to activate T cells in a FVIII-specific proliferation assay. These results indicate that N-glycosylation of FVIII can have significant impact on its immunogenicity.

Introduction. Inhibitor formation remains the major complication with treatment of hemophilia A (HA). Inhibitors, neutralizing antibodies specific to factor VIII (FVIII), form in 30% of patients during FVIII replacement therapy (Darby et al., 1977-99. J Thromb Haemost. 2004, 2(7): 1047-1054; and Hoyer and Scandella. Semin Hematol. 1994, 31(2 Suppl 4): 1-5). Many factors contribute to the induction of inhibitors (Lacroix-Desmazes et al., Front Immunol. 2019, 10:2991), however, much is still unknown about the key risk factors and the associated induction mechanism including why inhibitor formation only occurs in some patients but not in others. FVIII is a plasma glycoprotein. During processing in the endoplasmic reticulum (ER) and Golgi, sugar residues and oligosaccharide chains are covalently attached to the amino acids of its polypeptide chain. The differences in post-translational modifications, such as glycosylation may contribute to the differences in immune responses among patients.

In the last few years there have been multiple epidemiological studies performed highlighting the differential immunogenicity of FVIII products used in the treatment of HA patients (Lai et al., Blood. 2017, 129(24): 3147-3154; and Peyvandi et al., J Thromb Haemost. 2018, 16(1): 39-43). It was found that there is a 1.87 fold increase in inhibitor risk associated with recombinant FVIII (rFVIII) products compared to plasma-derived (pdFVIII) and a 1.6 fold increase in inhibitor risk associated with second generation baby hamster kidney (BHK) cell derived rFVIII compared to third generation Chinese hamster ovary (CHO) cell derived rFVIII (Lai et al., Blood. 2017, 129(24): 3147-3154). One hypothesis that may explain these differences is glycosylation since the glycan structures on each product differ based on the cell types they are derived from (Lai et al., Haematologica. 2018, 103(11): 1925-1936; and Krishnamoorthy et al., Cell Immunol. 2016, 301:30-39). Further studies are needed to understand and elucidate the mechanisms of inhibitor development, particularly the effects of glycosylation on FVIII immunogenicity that could lead to different responses in patients.

Multiple studies were recently performed analyzing the glycosylation of various pdFVIII and rFVIII products (Kannicht et al., Thromb Res. 2013, 131(1): 78-88; Canis et al., J Thromb Haemost. 2018, 16(8): 1592-1603; and Qu et al., PLoS One. 2020, 15(5): e0233576). Most N-linked glycosylation was detected in the B-domain of FVIII, with only four sites identified outside of the B-domain: N41 and N239 in the A1 domain, N1810 in the A3 domain, and N2118 in the C1 domain (Kannicht et al., Thromb Res. 2013, 131(1): 78-88; and Qu et al., PLoS One. 2020, 15(5): e0233576). Furthermore, while the locations of N-glycosylation sites remain the same in rFVIII and pdFVIII, the composition of glycoforms and occupancy at each site varies greatly from product to product (Canis et al., J Thromb Haemost. 2018, 16(8): 1592-1603; and Qu et al., PLoS One. 2020, 15(5): e0233576).

The development of inhibitors to FVIII is a T-cell dependent process and interactions between glycans and antigen presenting cells (APCs) have been observed. Sialic acids are known to interact with sialic acid-binding Ig-type lectins (siglecs) (Perdicchio et al., Proc Natl Acad Sci USA. 2016, 113(12): 3329-3334) including Siglec-5, an inhibitory receptor (Pegon et al., Haematologica. 2012, 97(12): 1855-1863), as well as asialoglycoprotein receptor (ASGPR) (Bovenschen et al., J Thromb Haemost. 2005, 3(6): 1257-1265) to protect FVIII from endocytosis. High mannose glycans on FVIII have been observed to interact with mannose-specific receptors on dendritic cells (DCs) in vitro, potentially leading to FVIII endocytosis (Dasgupta et al., Proc Natl Acad Sci USA. 2007, 104(21): 8965-8970). Given the research suggesting the potential roles played by different types of glycans (Perdicchio et al., Proc Natl Acad Sci USA. 2016, 113(12): 3329-3334; Bovenschen et al., J Thromb Haemost. 2005, 3(6): 1257-1265; Dasgupta et al., Proc Natl Acad Sci USA. 2007, 104(21): 8965-8970; and Pereira et al., Front Immunol. 2018, 9:2754), whether elimination of N-glycans at the four aforementioned sites in the A and C domains of BDD-FVIII would induce changes in the immune responses in a HA murine model when compared to WT BDD-FVIII in a gene therapy setting was explored.

Methods. Mutagenesis of N-linked glycans. Mutagenesis of the BDD-FVIII cDNA in the liver-specific plasmid (pBS-HCRHPI-hFVIII) (Miao. Adv Genet. 2005, 54:143-177) was carried out using the Agilent QuikChange II XL Mutagenesis kit (Agilent Technologies, Inc). A substitution was made swapping an Asparagine (N) at the site of interest with a Glutamine (Q) residue to eliminate N-glycosylation at the specified site. FVIII N to Q variants were created at sites N41, N239, N1810, and N2118 on the BDD-FVIII plasmid, respectively. Primers used were listed in FIG. 1.

Mice. All the experimental mice were housed in a specific pathogen-free (SPF) facility in Seattle Children's Research Institute according to the animal care guidelines of the National Institutes of Health and Seattle Children's Research Institute. The experimental protocols used in this example were approved by the Institutional Animal Care and Use Committee of Seattle Children's Research Institute.

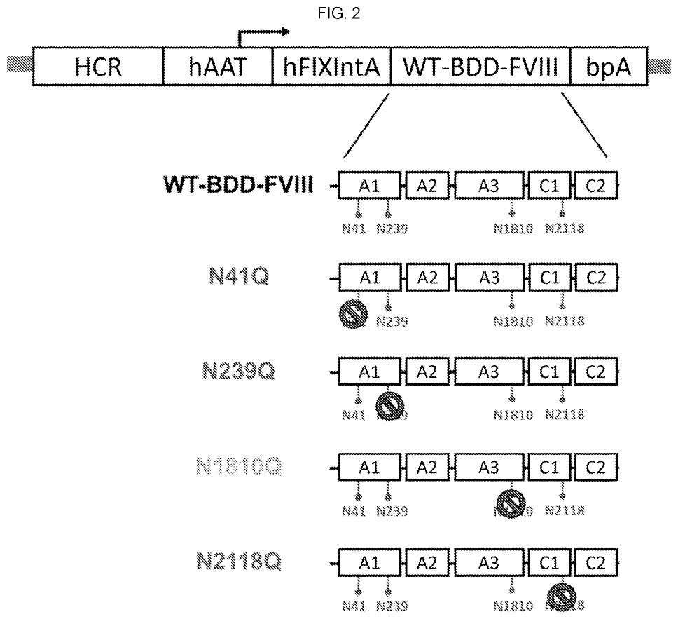

Delivery of plasmids carrying BDD-FVIII and its glycosylation variants genes into HA mice. Male HA mice with a FVIII exon 16 knockout in an Sv129/BL6 mixed background between the ages of 8-14 weeks were used in all experiments. Mice were injected with liver-specific plasmids carrying BDD-FVIII and its glycosylation variants genes (FIG. 2) via hydrodynamic injection at a concentration of 25 μg/mL and a volume (ml) equal to 9% the body weight of the mouse (g). A second challenge (SC) of the same plasmids via hydrodynamic injection was performed on Day 86 to elicit robust secondary immune responses. Blood was collected by retro-orbital bleed in one-tenth volume of 3.8% sodium citrate periodically following plasmid injections.

Adeno-associated viral vector (AAV) production, purification, and delivery. AAV-BDD-FVIII and AAV-2118Q-FVIII were produced by the triple plasmid transfection system in HEK 293 cells. Cell line, AAV production and determination of vector titers are described in the Supplemental Methods. AAVs were administered into HA mice at a dosage of 1×10¹² vg/mouse via tail vein injection.

Measurement of FVIII functional activity and antigen levels, and anti-FVIII inhibitory antibodies. Post hydrodynamic injection of FVIII plasmids into mice, the coagulant activity of FVIII, FVIII: C, was measured by a one-stage, activated partial thromboplastin time (aPTT)-based assay using a Stago® Compact Max instrument (Chen et al., Front Immunol. 2020, 11:638; and Chen et al., Mol Ther Nucleic Acids. 2020, 20:534-544). FVIII inhibitors were measured using Bethesda assay (Chen et al., Front Immunol. 2020, 11:638; and Chen et al., Mol Ther Nucleic Acids. 2020, 20:534-544). Measurement of FVIII antigen levels (FVIII:Ag) and total anti-FVIII IgG were examined by ELISA as described in Supplemental Methods.

CD4⁺ T-cell Proliferation Assay. Mice with high titer anti-FVIII inhibitors were generated as described in the Supplemental Methods. Mouse splenocytes were isolated and stained with BD Horizon™ violet cell proliferation dye 450 (VPD450) (BD Biosciences). The labeled splenocytes were cultured and stimulated by adding FVIII as a positive proliferation control, Factor IX as a nonspecific antigen control, or peptides, either glycosylated or non-glycosylated, corresponding to site N2118. Mannan (0.1, 1, and 10 μM, respectively) was added in mannan inhibition experiments. After 96 hours, cells were stained with anti-mouse CD4 and Fixable Viability Stain (Fisher Scientific) and analyzed by flow cytometry (BDT LSR II analyzer (Fisher Scientific)). The enhancement of proliferation was calculated by subtracting the background levels in non-stimulated cells.

Synthesis of non-glycosylated and glycosylated peptides. Non-glycosylated peptides, 15 amino acids in length, corresponding to sequences including site N2118 were synthesized and provided by GenScript. Glycosylated peptides, including peptides with either a single N-acetyl-glucosamine (GlcNAc) attachment or a high mannose glycan (Man6GlcNAc2), were synthesized in house. Peptides with GlcNAc were synthesized by using wang-resin through the Fmoc-strategy. To prepared mannosylated N-glycopeptides, Man6GlcNAc oxazoline was prepared as described in Umekawa et al., (J Biol Chem. 2008, 283(8): 4469-4479), and coupled to GlNAc-attached peptides. The detailed synthetic methods of glycosylated peptides were described in Supplemental Methods.

Statistical Analysis. All statistical analyses were carried out utilizing GraphPad Prism 7 software. The data was compared using one-way or two-way analysis of variance (ANOVA) or multiple t-tests. P-value's<0.05 were considered statistically significant.

Supplemental Methods. Measurement of total anti-FVIII IgG. 96 well flat bottom ELISA plates (Corning, Kennebunk, ME) were coated overnight at 4° C. with 0.44 U FVIII/well in coating buffer. Plates were then incubated with blocking buffer (1×Tris-Buffered Saline (TBS), 0.1% Tween-20, and 20% non-fat milk for 1 hour at 37° C. Plates were washed three times with wash buffer I 1×TBS and 0.1% Tween-20) between incubation steps. Diluted plasma samples in blocking buffer were then added to the plate to incubate for 2 hours at 37° C. Plates were then incubated for 1 hr at 37° C. with goat anti-mouse IgG conjugated with HRP (Thermo Scientific). Subsequently Enhanced K-Blue (TMB) substrate (Neogen, Lexington, KY) was added and color was allowed to develop before stopping the reaction using 2N H2SO4. Concentrations of anti-FVIII IgG were evaluated by interpolation against the linear range on a standard curve created using mouse isotype control (Thermo Scientific) that was run in duplicate alongside samples on all plates.

FVIII antigen (FVIII:Ag) ELISA. FVIII:Ag levels in mouse plasma post hydrodynamic injection with FVIII plasmids were determined by ELISA using murine anti-FVIII antibody (GMA-8020, Green Mountain Antibody, Burlington, VT) and biotin-labeled murine anti-FVIII antibody (GMA-8015, Green Mountain Antibody, Burlington, VT). A standard curve was generated from serially diluted normal human plasma upon which samples were interpolated to determine FVIII antigen levels following the the previously described protocol (Chen et al., Mol Ther Nucleic Acids. 2020, 20:534-544).

Generation of hemophilia A mice with high-titer inhibitors. Anti-FVIII inhibitor mice were generated by priming the mice with 3 units (U) of recombinant full-length FVIII in 200 uL PBS 3× a week for four weeks. Mice were then sacrificed on week 5 for spleen collection or boosted one-week prior to spleen collection. Blood was drawn via retro-orbital bleed on the day of sacrifice in the same manner as before in order to measure IgG titer via enzyme-linked immunosorbent assay (ELISA).

Detailed synthetic methods of glycosylated peptides. The synthesis of peptides with GlcNAc was performed on a CEM Liberty Blue Peptide Synthesizer with microwave-assisted protocols. HBTU was used as activator and DIEPA was used as base, piperidine in DMF (20%, v/v) was used as deprotected reagent. Double coupling was performed with microwave at 50° C. Each coupling cycle took 10 min. Cocktail of TFA/TIS/Dodt/H2O (92.5:2.5:2.5:2.5) was used to cleave peptides off from resin. Additionally, 5% aqueous hydrazine was used to deprotect the protected acetyl group to free the GlcNAc residue on peptides. Crude peptides attached with one GlcNAc were purified by using a Xbridge peptide BEH C18 HPLC column (5 μm, 10 mm×250 mm) and analyzed by an analytical Xbridge peptide BEH C18 column (5 μm, 4.6 mm×250 mm). Purified GlcNAc-attached peptides were characterized by HPLC and LC-MS/MS. The HPLC condition is as following: solvent A (H2O with 0.1% TFA) and solvent B (ACN with 0.1% TFA) with gradient elution from 5% to 20% solvent B in 30 mins in a flow rate of 1 mL/min for analysis and 4 mL/min for purification.

To prepared mannosylated N-glycopeptides, Man6GlcNAc was purchased from NatGlycan LLC (Atlanta, GA, USA). Man6GlcNAc oxazoline was prepared as previously described in Umekawa et al., (J Biol Chem. 2008, 283(8): 4469-4479), and dissolved in 100 mM MES buffer (pH 7.0) together with GlNAc-attached peptides, then adequate amount of Endo-A-N171A was added to the reaction mixture and incubated at 30° C. for 10 mins. After the reaction was quenched, the mixture containing the target compound was subjected to characterization by HPLC with an Xbridge Peptide BEH C18 column (3.5 μm, 4.6 mm×250 mm) column and purified by an Xbridge Peptide BEH C18 column (5 μm, 10 mm×250 mm). The HPLC condition is as following: solvent A (H2O with 0.1% TFA) and solvent B (ACN with 0.1% TFA) with gradient elution from 5% to 20% solvent B in 30 mins in a flow rate of 1 mL/min for analysis and 4 mL/min for purification.

Cell line. HEK293 cell line (a human embryonic kidney cell line) was purchased from ATCC. The cells were cultured in DMEM supplemented with 10% fetal bovine serum, 100 μg/mL penicillin, and 100 units/mL streptomycin (Invitrogen, Carlsbad, CA), and maintained in a humidified 37° C. incubator with 5% CO2.

AAV production and purification. HEK293 cells were seeded in 2-L roller bottles 24 hour prior to transfection. The three plasmids of pH28, pFA6 and pAAV-BDD-FVIII or pAAV-N2118Q-FVIII were delivered at the mole ratio of 1:1:1 into 293 cells using PolyJet™ DNA In Vitro Transfection Reagent (SignaGen Laboratories) when transfection. The old media were replaced with fresh DMEM containing 2% of FBS at 12 hours after transfection. The medium collected at 96 hours post transfection, and precipitated with 40% of PEG (finial concentration 8%) overnight at 4° C. Next, it was centrifugated at 3800 rpm, 40 min at 4° C., resuspended in HBS buffer (pH 8.0) and treated with DNase I and RNase I at 37° C. for one hour. AAV vectors were purified by iodixanol gradient ultracentrifugation. AAV vector fractions were extracted and exchanged in 0.2M NaCl-Phosphate Buffered Saline (PBS, NaCl 137 mM, KCl 2.7 mM, Na2HPO4 10 mM, KH2PO4 1.8 mM, pH 7.2). Vector genome titers were determined by quantitative real-time PCR (qPCR), with vector titers expressed as vg/ml.

qRT-PCR assay for AAV vector titer. To detect AAV vector titer, the sample was treated following below protocol:

10 ul of AAV vector was added into 90 ul of DNase solution containing DNase I (1 U/mL), incubated for 30 min at 37° C., adding 1 uL of 0.5 M EDTA (to a final concentration of 5 mM), and subsequently heated for 10 min at 75° C. to cease DNase I activity. 50 ul of lysis buffer containing proteinase K (40 mg/mL) was added, then incubated for 1 h at 56° C., and finally heated for 10 min at 95° C. The copy numbers of vector genomes released were quantified by real-time PCR and expressed in vector genomes/milliliter. The primers used are targeting the FVIII gene, forward 5′-TGACCCTGAAGTTCATCTGC-3′ (SEQ ID NO: 9), reverse 5′-GAAGTCGTGCTGCTTCATGT-3′ (SEQ ID NO: 10).

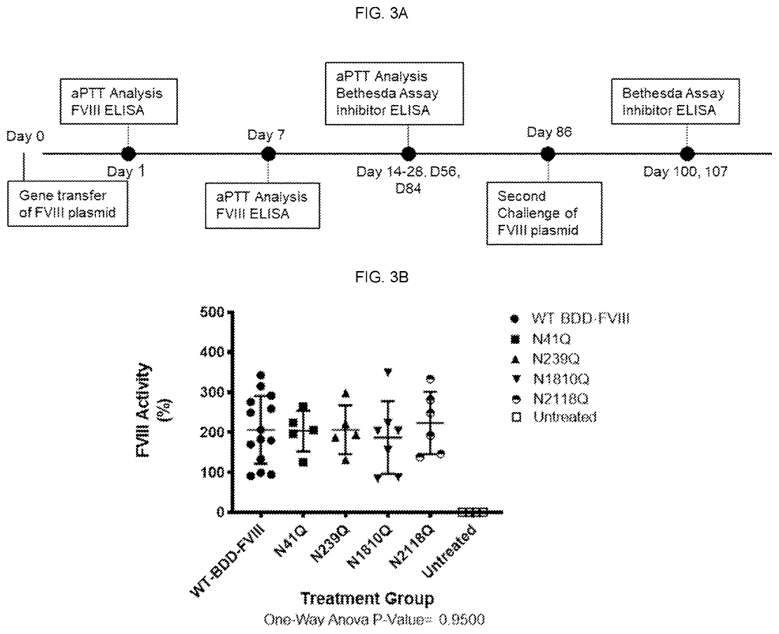

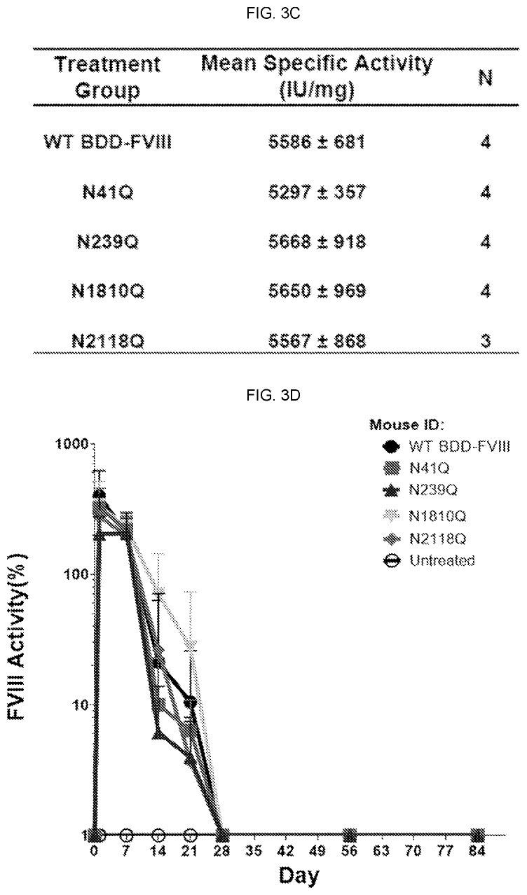

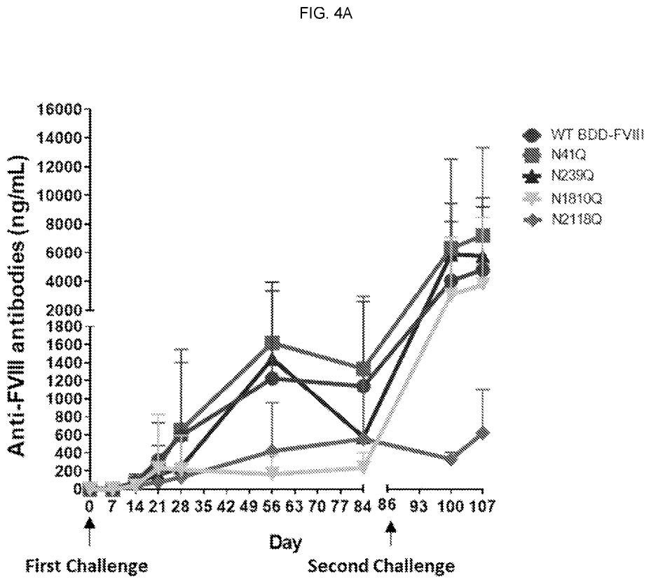

Results. Comparison of FVIII expression resulting from the wild type and glycosylation variant genes. FVIII variants were created through site directed mutagenesis of the cDNA of a WT BDD-FVIII plasmid at each of the four N-glycosylation sites in the A and C domains by substituting the N residue with a Q residue (pBS-HCRHPI-hFVIII) (Miao, Adv Genet. 2005, 54:143-177) (FIGS. 1 and 2). These variants could effectively eliminate glycosylation at each site. The plasmids carrying the genes encoding BDD-FVIII and glycosylation variants were then hydrodynamically injected, respectively, into HA mice. FVIII functional activity levels in treated mouse plasma were evaluated one-week post-injection (FIG. 3A). Mice injected with the WT BDD-FVIII plasmid had an average activity of 206±84% and mice injected with N41Q, N239Q, N1810Q, and N2118Q FVIII variant plasmids had average activities of 203±50%, 206±60%, 187±91%, and 223±77%, respectively (FIG. 3B). No statistically significant differences in FVIII activities between the variants and WT BDD-FVIII were observed by a one-way ANONVA analysis (p-value close to 1). FVIII:Ag levels were determined by FVIII ELISA. The specific activities of FVIII produced in all treated groups also showed no statistical differences (FIG. 3C). Taken together, these results indicated that mutation in each of these sites likely did not induce any significant conformational changes in the protein structure to affect FVIII functional activity and production in vivo. FVIII functional activities were monitored weekly for the duration of the experiment via aPTT analysis (FIG. 3A) and the % FVIII in circulation dropped to undetectable levels in all treatment groups by Day 28 (FIG. 3D).

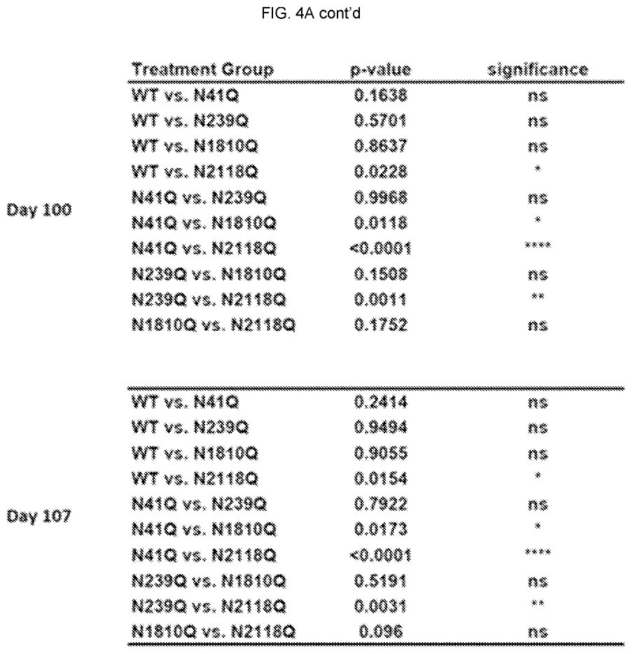

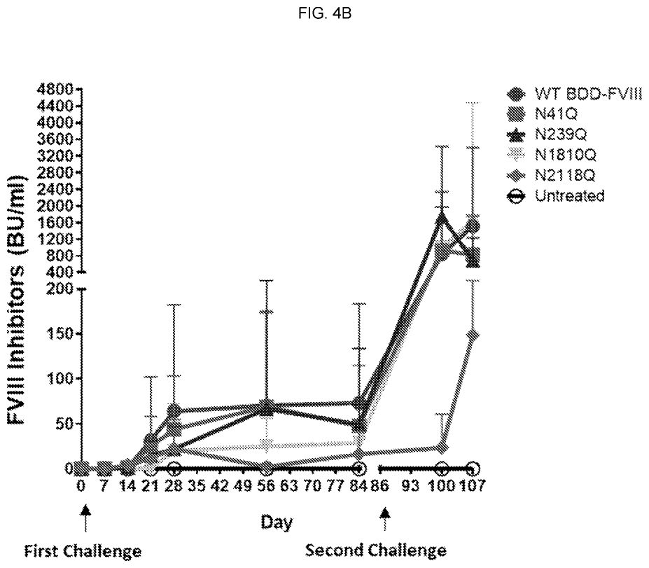

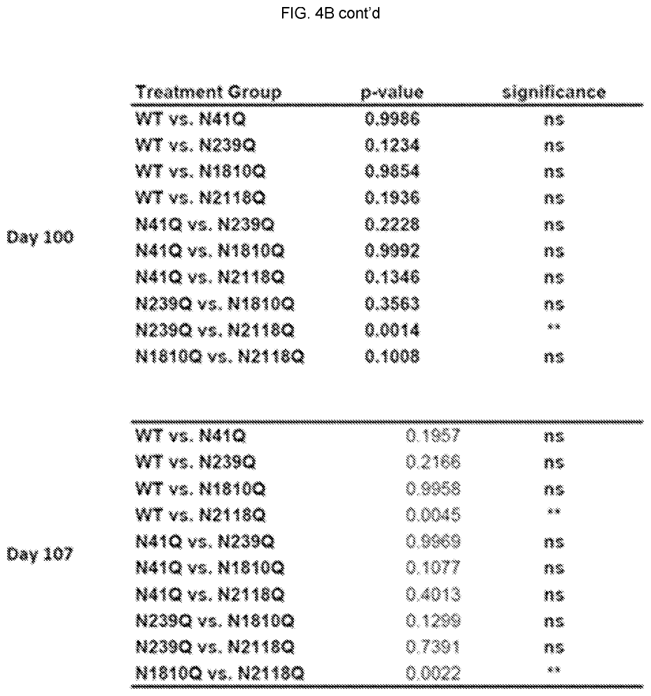

Evaluation of the impact of N-glycosylation on the immunogenicity of FVIII following plasmid-mediated gene transfer. After confirming that the mutagenesis experiments to eliminate individual sites of glycosylation had not affected FVIII functional activity and stability in vivo and given the drop in FVIII functional activity by day 14, the inhibitor responses were explored in the treated mice. HA mice were injected hydrodynamically with WT BDD-FVIII plasmid or one of the four variant plasmids. Following plasmid transfer, anti-FVIII IgG measured by ELISA appeared at low levels (<100 ng/ml) in all injection groups by day 14, then began to rise and peak around day 56, at which point some N1810Q and N2118Q groups had lower antibody levels compared to the other three groups. Subsequently, a second plasmid challenge was performed on day 84, via hydrodynamic injection. While all other groups experienced a drastic increase in anti-FVIII levels after the second challenge, N2118Q group did not (FIG. 4A). Bethesda assays were also performed on the same samples and time points to examine the inhibitor responses (FIG. 4B). These results parallels those of antibody responses with N1810Q and N2118Q groups having lower inhibitor titers compared to other group on day 56 and N2118Q group having significantly lower inhibitor titer post-second challenge. Taken together, these assays confirmed that when site N2118 is mutated to eliminate glycosylation, there is a decrease in inhibitor development compared to WT BDD-FVIII and the other N-glycosylation variants. It is also worth noting that there is a trend that anti-FVIII antibody levels in N41Q and N239Q groups were higher than in the WT BDD-FVIII group.

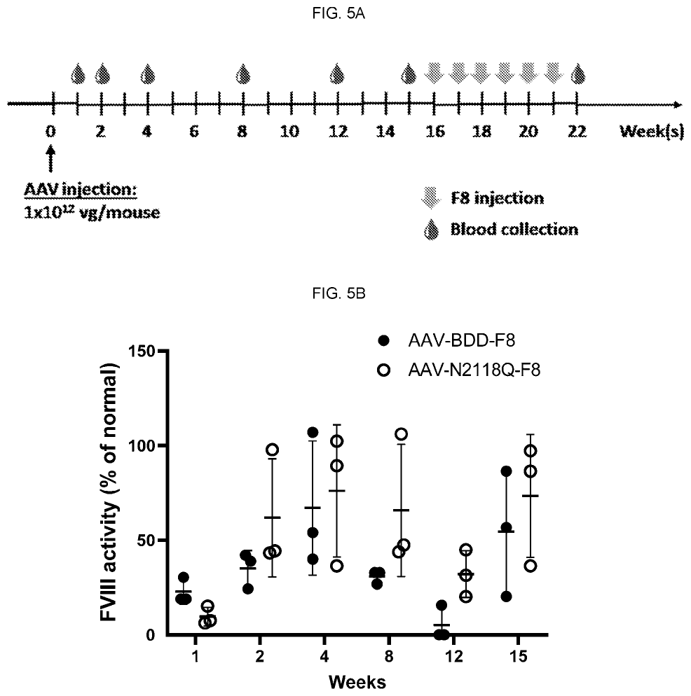

Evaluation of the impact of N-glycosylation on the immunogenicity of FVIII following AAV-mediated gene delivery. Groups of HA mice (n=3/group) received AAV-BDD-FVIII and AAV-N2118Q-FVIII at a dosage of 1×10¹² vg/mouse via intravenous injection (FIG. 5A). FVIII activities were detected at week 1 and gradually increased to average of 70% of normal plasma FVIII functional activity (100%) on week 4. In AAV-BDD-FVIII treated mice, FVIII activities dropped to lower levels on week 8 and continued to decline to <10% (1 mouse) or undetectable levels (2 mice) on week 12 (FIG. 5B). In the two mice with undetectable FVIII levels, very low levels of anti-FVIII antibodies were detected in ELISA but no inhibitor titers by Bethesda assay. In AAV-2118Q-FVIII treated mice, FVIII expression remained at similar levels on week 4 and 8, however moderately dropped to lower levels at week 12. Interesting, both groups of mice had FVIII levels reverted to higher levels on week 15, suggesting potential tolerance induction between week 12 and week 15. When these experiments were repeated with a new batch of AAVs, the same trend was observed. Next, the groups of mice were challenged with 5 U of FVIII weekly for six weeks. One week after the final challenge, inhibitor titers were examined (FIG. 5C). The prevalence of inhibitor development of AAV-BDD-FVIII and AAV-N2118Q-FVIII injected mice was 100% and 33%, respectively (FIG. 5C), indicating mice treated with AAV-2118Q-FVIII were more tolerant to FVIII compared with mice treated with AAV-BDD-FVIII.

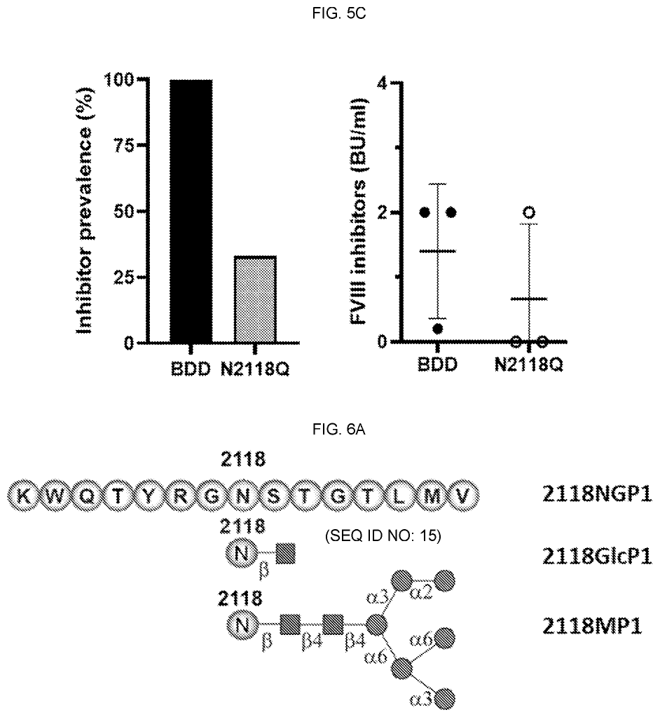

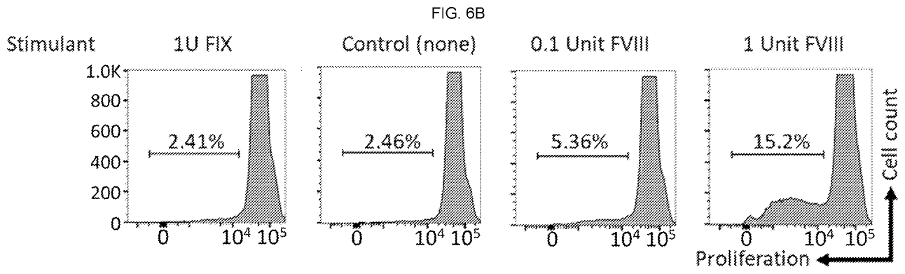

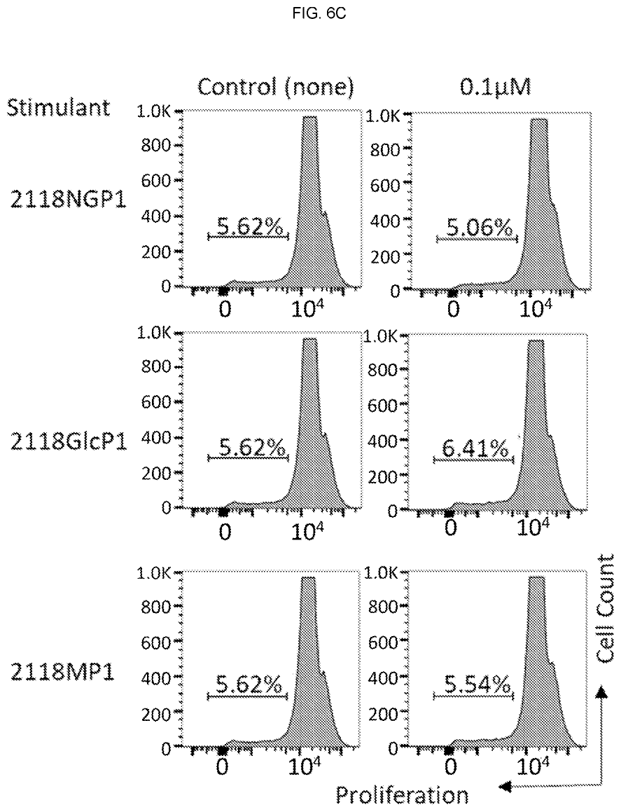

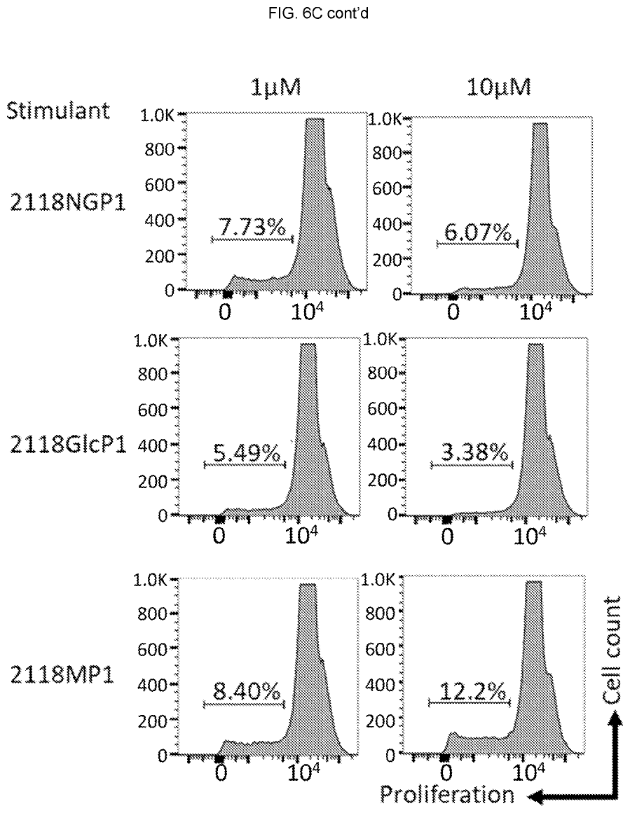

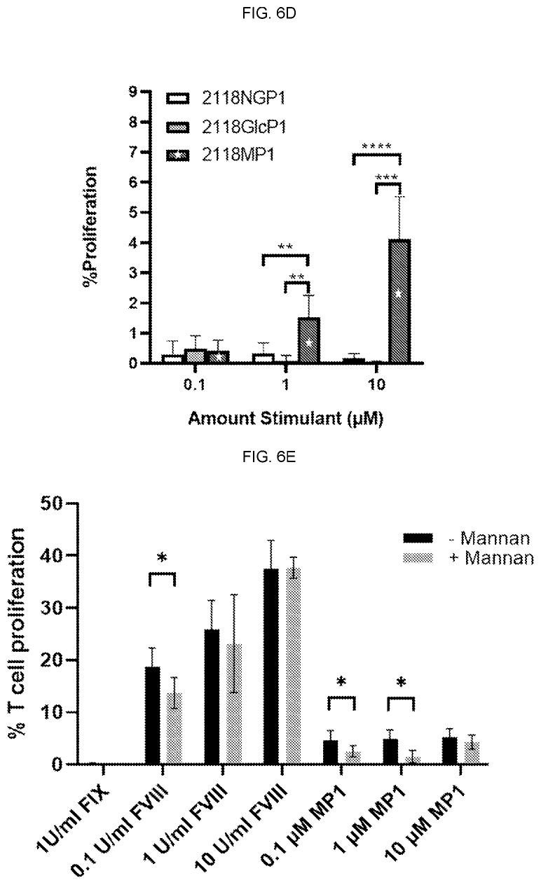

Identification of a potential T cell glycopeptide epitope surrounding site N2118 of FVIII. Given the reduced immune response in mice treated with the FVIII variant N2118Q seen in both the IgG ELISA and Bethesda assays, the specific role of glycosylation at this site in interactions with the immune system were explored. As the development of inhibitors to FVIII is known to be a T-cell dependent process, an in-vitro proliferation experiment was performed using a non-glycosylated (2118NGP1) peptide, a GlcNAc-attached peptide (2118GlcP1) and a mannosylated peptide (2118MP1) corresponding to the amino acid (aa) sequence around N2118 site of FVIII with the asparagine located centrally within the 15-mer peptide (Peptide 1: K₂₁₁₁WQTYRGN₂₁₁₈STGTLMV₂₁₂₅) to investigate CD4⁺ T-cell responses (FIG. 6A).

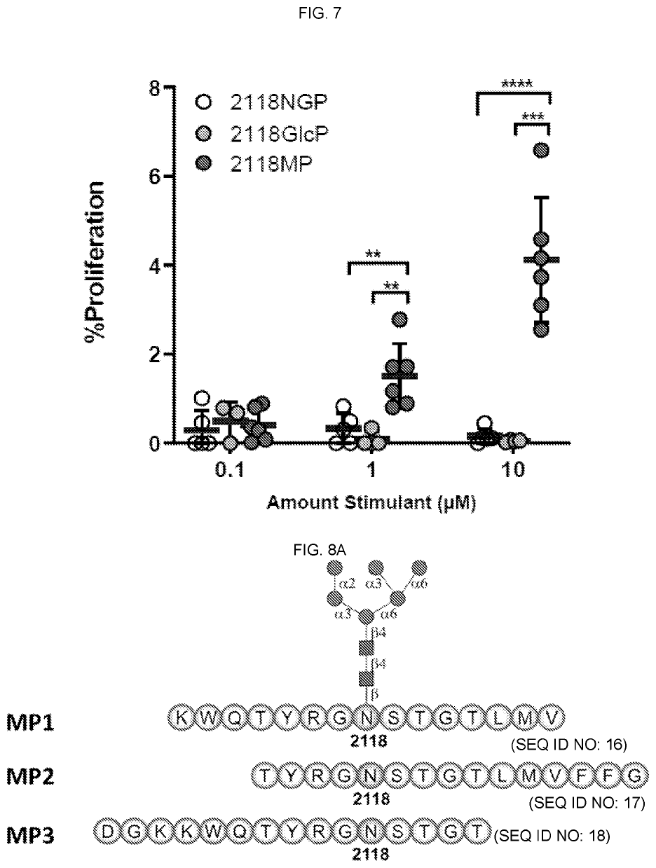

These peptides were examined for their ability to induce FVIII-specific T cell proliferation using splenocytes isolated from FVIII-primed HA mice with high-titers of anti-FVIII IgGs. A dose-dependent increase was observed in response to increasing doses of FVIII in the FVIII-specific CD4⁺ T-cell proliferation assay and no response was observed in the presence of factor IX (FIX) as a nonspecific antigen control (FIG. 6B). Having confirmed the FVIII specificity of cell proliferation, CD4⁺ T-cell proliferation was looked at in response to stimulation with either 2118NGP1, 2118GlcP1 or 2118MP1. Representative runs are shown in FIG. 6C. Multiple runs were performed under the same conditions and the summary of data was shown in FIGS. 6D and 7. In response to the increased doses of high mannose N2118 peptide (2118MP1) from 0.1 μM to 10 μM, CD4⁺ T-cells showed increased levels of proliferation with 4.2±1.3% at 10 μM, while 2118NGP1 and 2118GlcP1 had negligible effects on cell proliferation. These results indicated that the specific mannosylated peptide 2118MP1 may encompass a T-cell responsive glycopeptide epitope region of FVIII as opposed to the unglycosylated version.

Next, mannan inhibition experiments were performed to verify the effect of mannosylation of FVIII on induction of T cell responses using the FVIII-specific T cell proliferation assay. It was found that T cell proliferation was only partially inhibited in response to 0.1 U/ml FVIII and no inhibition was observed at 1 U/ml or 10 U/ml FVIII. However, mannan inhibited T cell proliferation in response to all three concentrations of 2118MP1(0.1, 1 & 1 μM). These data further confirmed that mannosylation at 2118 site is responsible for T cell activation.

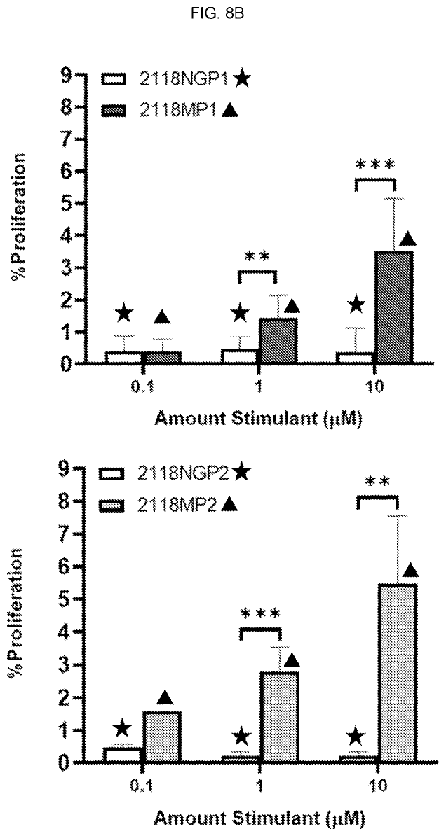

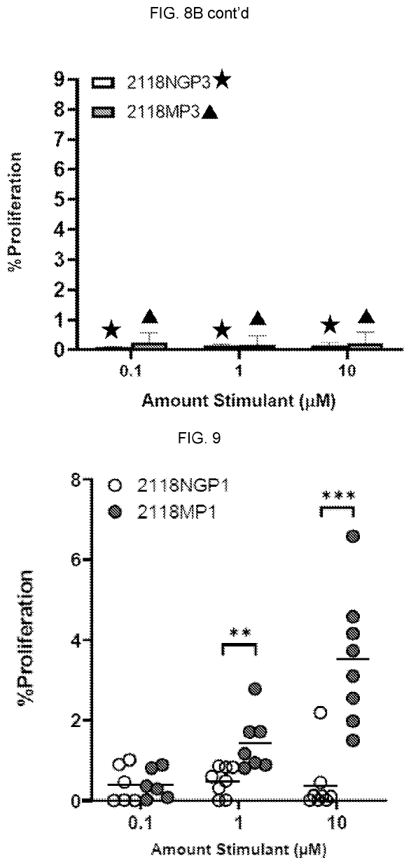

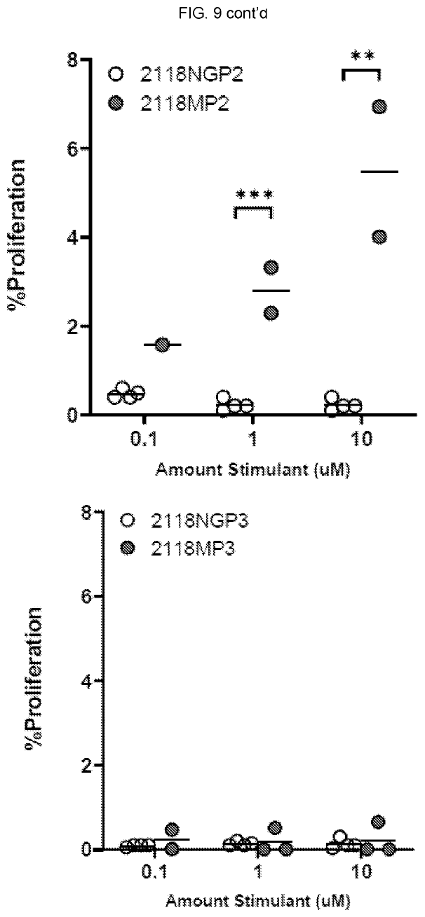

Screening of glycopeptide epitope surrounding N2118 μsing overlapping peptides. To further narrow down the region of FVIII responsible for eliciting an increased immune response in the presence of a mannosylated site N2118, overlapping non-glycosylated and glycosylated (Man6) peptides with site N2118 shifted towards either the N- or C-terminus of the 15-mer peptide (Peptide 2: T₂₁₁₄YRGN₂₁₁₈STGTLMVFFG₂₁₂₈; Peptide 3: D₂₁₀₈GKKWQTYRGN₂₁₁₈STGT₂₁₂₂) were synthesized. These peptides were analyzed to compare proliferation with mannosylated and non-glycosylated peptide 1 (K₂₁₁₁WQTYRGN₂₁₁₈STGTLMV₂₁₂₅), where the N-glycosylation site is centrally located (FIG. 8A). Compared to 2118MP1 with enhanced proliferative CD4⁺ T-cell responses relative to its non-glycosylated counterpart 2118NGP1 (FIGS. 8B and 9), stimulation with 2118MP2 resulted in even higher levels of CD4⁺ T-cell proliferation than with 2118MP1, at 2.9±0.6% at 1 μM and 5.5±2.0% at 10 μM, and with significantly increased proliferation in comparison to 2118NGP2 (FIGS. 8B and 9). On the other hand, 2118MP3 showed little to no proliferation in comparison to the other mannosylated peptides and its non-glycosylated counterpart 2118NGP3 (FIGS. 8B and 9). Proliferation of CD4+ T-cells in the presence of all three non-glycosylated peptides (NGP's) were either at or near background levels. These data indicated that the essential sequence of the potential T-glycopeptide epitope region is located in the overlapping region of 2118MP1 and 2118MP2.

Discussion Post-translational modifications of proteins, such as glycosylation, and the possible effects on the immune response in HA have become of interest in recent years. Recent studies have determined the location of glycosylation sites and the composition of glycan structures in different FVIII products (Kannicht et al., Thromb Res. 2013, 131(1): 78-88; Canis et al., J Thromb Haemost. 2018, 16(8): 1592-1603; and Qu et al., PLoS One. 2020, 15(5): e0233576). Sialic acids as the terminal sugar may act as protective sugar moieties from mounting immune responses while high-mannose structures may act as immunogenic moieties (Perdicchio et al., Proc Natl Acad Sci USA. 2016, 113(12): 3329-3334; Dasgupta et al., Proc Natl Acad Sci USA. 2007, 104(21): 8965-8970; and Pereira et al., Front Immunol. 2018, 9:2754). Given that the largest barrier to successful FVIII treatment remains to be inhibitor development in patients, the roles of specific sites of N-glycosylation on FVIII immunogenicity were investigated through the removal and addition of glycans in a HA mouse model.

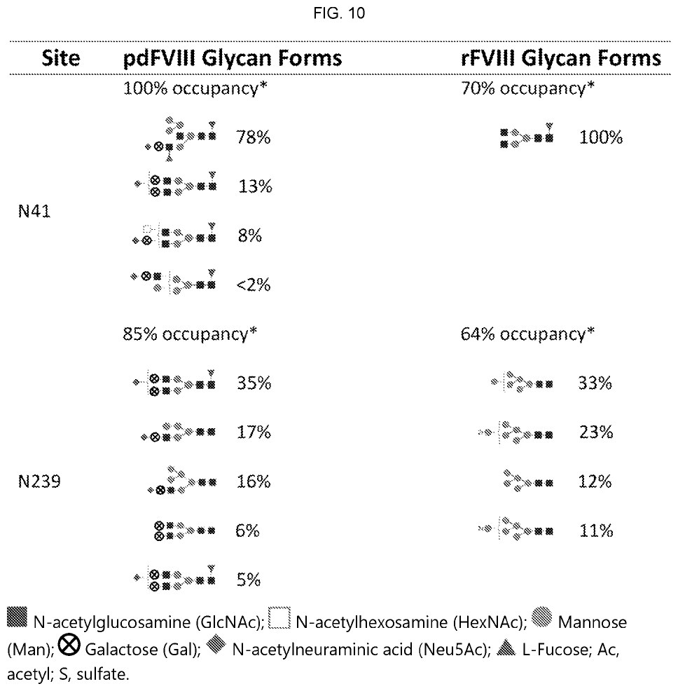

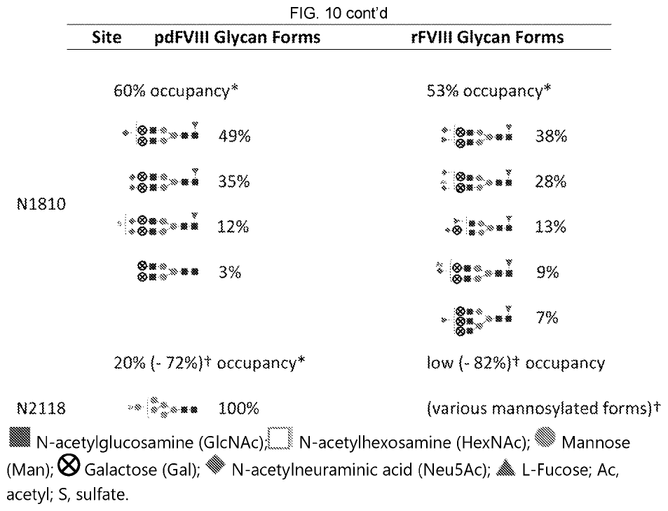

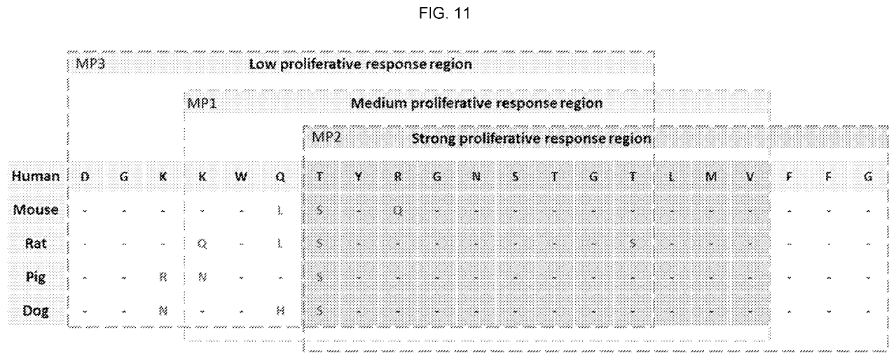

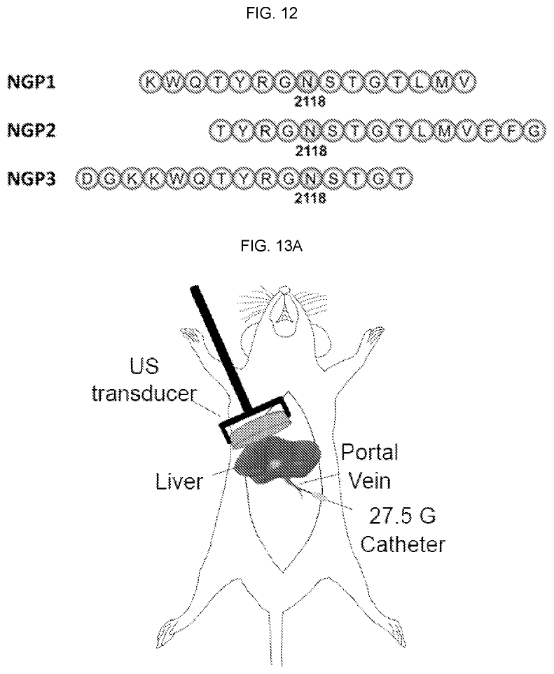

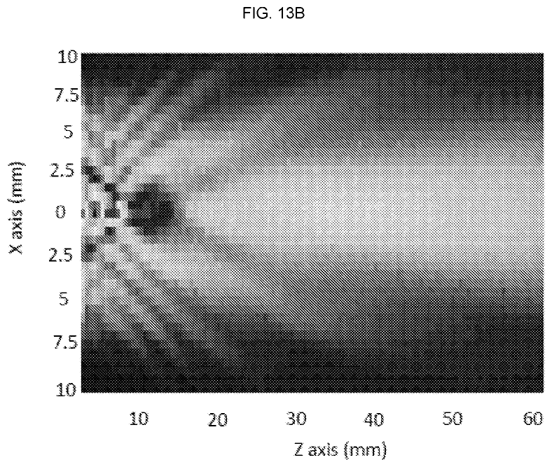

N-linked glycosylation occurs in the presence of an N—X-serine(S)/threonine (T) (where X is not proline) consensus sequence and is the most common form of glycosylation in human cells (Lyons et al., Front Pediatr. 2015, 3:54). There are three classes of N-linked glycans: complex-type, high-mannose type, and hybrid-type (Lyons et al., Front Pediatr. 2015, 3:54). Five consensus N—X—S/T sequences in FVIII have been identified outside of the B-domain (Kannicht et al., Thromb Res. 2013, 131(1): 78-88; Canis et al., J Thromb Haemost. 2018, 16(8): 1592-1603; and Qu et al., PLoS One. 2020, 15(5): e0233576) including N41 and N239 in the A1 domain, N582 in the A2 domain, N1810 in the A3 domain and N2118 in the C1 domain (FIG. 2). However, no glycosylation was detected on N582 in either pdFVIII or rFVIII (Kannicht et al., Thromb Res. 2013, 131(1): 78-88; and Qu et al., PLoS One. 2020, 15(5): e0233576). Detailed analysis (Qu et al., PLoS One. 2020, 15(5): e0233576) illustrated a comparison of the percentages of various types of N-glycans on pdFVIII and rFVIII (FIG. 10), revealing dramatic differences on the four N-glycosylation sites outside the B-domain between pdFVIII and rFVIII. Most notably, in pdFVIII, N2118 was occupied by high-mannose glycans, whereas N41, N239 and N1810 sites were occupied mostly by sialylated complex and hybrid-type glycoforms. On the other hand, in rFVIII, few or high-mannose glycans were found in N2118, whereas N41 was occupied with sialylated glycans and N239 with high-mannose ones. It should also be noted that all 4 N-glycosylation sites are surface exposed residues and thus able to interact with immune moieties in vivo (Shen et al., Blood. 2008, 111(3): 1240-1247; Stoilova-McPhie et al., Blood. 2002, 99(4): 1215-1223).

Glycosylation of proteins can not only influence its interactions with immune cells, but also contribute to the stability and function. The mutagenesis of sites N41, N239, N1810, and N2118 from N to Q were first examined to see if they would affect FVIII functional activity and/or accelerate an immune response and FVIII clearance. The homologous Q residue is chosen since it is never glycosylated due to the requirement of the special N—X—S/T structure to form N/D-turn conformation to undergo glycosylation (Imperiali et al., Curr Opin Chem Biol. 1999, 3(6): 643-649). It was reported that fully deglycosylated FVIII exhibited significantly decreased activity as well as decreased interactions with phosphatidylserine containing membranes (Kosloski et al., AAPS J. 2009, 11(3): 424-431). Nevertheless, previous reports showed that a single mutation of these four N-glycosylation sites either decreased or maintained the same antigen levels and activity of FVIII (Wei et al., Biochem J. 2018, 475(5): 873-886; Selvaraj et al., Blood. 2011, 118(21): 2238; Delignat et al., Front Immunol. 2020, 11:393; and Ito et al., J Thromb Haemost. 2022, 20(3): 574-588). In this example, following delivery of plasmids carrying the WT BDD-FVIII and the four glycosylation variants, mice produced similar levels of FVIII expression and specific activities, based on aPTT and FVIII:Ag ELISA data. These results indicated that the removal of each of these four N-glycosylation sites, respectively, does not result in significant conformational changes which would affect protein activity/stability in these in vivo mouse models. These gene therapy treated mice provided the opportunity to focus on examining the impact of glycosylation on FVIII immunogenicity.

Given that anti-FVIII inhibitor development is the biggest obstacle standing in the way of successful FVIII treatment in patients, it is important to consider the impact that post-translational modifications such as glycosylation may have upon its immunogenicity. As shown in the results, mice treated with plasmids carrying WT BDD-FVIII and the four glycosylation variants all developed anti-FVIII inhibitors. Following first plasmid challenge, groups of mice treated with N1810Q and N2118Q showed a trend of decreased inhibitor responses to FVIII compared to mice treated with WT BDD-FVIII plasmid, whereas no significant difference in inhibitor responses were observed between N41Q, N239Q and WT BDD-FVIII plasmid-treated mice. Subsequently, each group of mice was treated with the same plasmid a second time on day 84. Mice treated with N2118Q group showed statistically significant lower inhibitor responses compared with WT BDD-FVIII, whereas N41Q, N139Q and N1810Q groups had slightly increased or comparable responses with WT BDD-FVIII group. The reduction of inhibitor response by N2118Q FVIII variant is also demonstrated in AAV-mediated gene transfer experiments. As shown in FIG. 10, the glycoforms detected at N2118 site were all high-mannose-type glycans, whereas N41, N139 and N1810 sites were occupied 60-100% with complex and hybrid-type glycans with mostly terminal sialic acids. It is striking that high-mannose glycans on N2118 site had such a significant impact on the immunogenicity of FVIII. It is noteworthy that compared to pdFVIII, N41 lost the terminal sialic acid and N239 changed from complex and hybrid-type glycans to high-mannose glycans in rFVIII (FIG. 10). Whether these changes contribute to the higher immunogenicity of rFVIII warrants further investigation. Furthermore, the N2118Q variant could be used as a more tolerogenic FVIII compared to WT BDD-FVIII.

High-mannose glycans were postulated to facilitate the uptake of FVIII via the mannose receptors on dendritic cells (DCs) and macrophages (Delignat et al., Front Immunol. 2020, 11:393; and Repesse et al., J Allergy Clin Immunol. 2012, 129(4): 1172-1173, author reply 1174-1175); however, a controversial study showed conflicting data (Herczenik et al., J Allergy Clin Immunol. 2012, 129(2): 501-509, 509 e501-505). Mannan inhibition in a T cell proliferation assay was studied. Two steps could be involved in mannan inhibition, the first step is the entry of FVIII with mannosylated residues into APCs involving mannose receptors and the second step involved is the interaction of mannosylated peptide presented on APCs with the T cell receptor. It was shown that mannan only partially inhibited the proliferation rate in response to FVIII at low FVIII concentrations but not at higher FVIII concentrations, indicating there is another major pathway that govern the uptake of FVIII by APCs in mice, consistent with previous studies (Herczenik et al., J Allergy Clin Immunol. 2012, 129(2): 501-509, 509 e501-505; and Delignat et al., Haemophilia. 2012, 18(2): 248-254). Whether this is the same in human needs further investigation. While glycans can affect immunogenicity of proteins by presenting itself as a glyco epitope (Wang. J Proteomics Bioinform. 2014, 7(2)), it can also shield other epitopes from T cells (Gram et al., PLoS Pathog. 2016, 12(4): e1005550) and antibodies (Lavie et al., Front Immunol. 2018, 9:910), regulate presentation of neighboring epitopes (Li et al., J Immunol. 2009, 182(10): 6369-6378), or be a part of a specific peptide epitope (glycopeptide epitope) (Sun et al., Nat Commun. 2020, 11(1): 2550). Due to the significant impact of N2118Q mutation on FVIII immunogenicity in the gene therapy treated HemA mice, the possibility of a potential glyco or glycopeptide epitope was considered around the N2118 site. In order to test this hypothesis, three 15-mer peptides centered around N2118 (K₂₁₁₁WQTYRGN₂₁₁₈STGTLMV₂₁₂₅) were synthesized with or without GlcNAc, or Man6GlcNAc2 attached, respectively. The highly mannosylated (Man6GlcNAc2) peptide 2118MP1 showed significant stimulatory effect of FVIII-specific T cell responses, whereas the non-glycosylated 2118NGP1 and GlcNAc-attached 2118GIcP1 showed no stimulation. Next, additional overlapping peptides were made, and a strong stimulatory mannosylated peptide 2118MP2 (T₂₁₁₄YRGN₂₁₁₈STGTLMVFFG₂₁₂₈) with FVIII-specific T cell responses and a non-stimulatory mannosylated peptide 2118MP3 (D₂₁₀₈GKKWQTYRGN₂₁₁₈STGT₂₁₂₂) were found. Notably, none of the non-glycosylated peptides showed any stimulatory effect in the FVIII-specific proliferation assay. In addition, 2118MP2 encompasses the most conserved region among FVIII derived from different species, whereas 2118MP3 encompasses the least conserved region (FIG. 8). These results together suggest a potent T-cell specific glycopeptide epitope between K₂₁₁₁ to G₂₁₂₈ of FVIII. Thus, deletion of glycans in N2118 site can significantly reduce the immunogenicity of FVIII due to the elimination of a potent glycopeptide epitope for antigen presentation to interact with and activate T cells. These results also indicate that there is a specific glycopeptide epitope instead of a general glycoepitope surrounding the N2118 glycosylation site.

Recent studies (Batsuli et al., J Thromb Haemost. 2018, 16(9): 1779-1788; Hartholt et al., J Thromb Haemost. 2017, 15(2): 329-340; and Vollack et al., Scand J Immunol. 2017, 86(2): 91-99) showed that antibodies may influence de novo antibody formation following exposure to distinct FVIII proteins. FVIII glycosylation may also influence the innate-like B cell recognition of these glycan epitopes for initiation of antibody response (Zerra et al., Front Immunol. 2020, 11:905), especially when considering that naturally occurring antibodies may facilitate inhibitor formation as has recently been shown with xenoglycans (Arthur et al., Blood. 2022, 139(9): 1312-1317). These studies, in addition to the present example, provide an understanding of how FVIII glycosylation may influence antibody formation against FVIII in a variety of contexts.

In conclusion, this example examined the impact of post-translational modifications, specifically N-glycosylation on the immunogenicity of FVIII synthesized in vivo following gene transfer of FVIII plasmids. Four N-glycosylation sites outside the B-domain were examined. Three sites with predominantly sialylated complexes or hybrid glycans did not significantly alter the immunogenicity of FVIII, whereas N2118 with high-mannose glycans showed significant impact on FVIII immunogenicity. A potent T-cell specific glycopeptide epitope surrounding N2118 was identified and characterized in FVIII for the first time. These results can enhance the understanding of inhibitory antibody formation against FVIII and facilitate the development of a more tolerant FVIII molecule for replacement protein and gene therapy for hemophilia treatment.

Example 2. Inhibitor formation and immunogenicity of FVIII. In hemophilia A treatment, inhibitor formation is the major complication. Thirty percent of patients develop inhibitor during FVIII replacement therapy. Epidemiological studies showed higher incidence of inhibitor development in patients treated with FVIII expressed by baby hamster kidney (BHK) cells than Chinese hamster ovary (CHO) cells.

Patients treated with second generation FVIII products, FVIII expressed by BHK cells had 1.6 times higher risk to inhibitor formation than patients treated with third generation FVIII products. One explanation for the findings is the glycan structures on each product differ based on the cell types they are derived from. In this example, the roles of glycosylation in FVIII immune response were investigated.

Comparison of FVIII expression resulting from the wild type and glycosylation variant genes. Most N-linked glycosylation was detected on the B domain of FVIII with only four glycosylation sites outside of the B domain, including N41, N239, N1810 and N2118. To elucidate the effects of N-glycosylation on each of the four sites in FVIII immune responses, four FVIII variant plasmids were created. The plasmids have amino acid substitution from asparagine to glutamine at each of the four glycosylation sites. The mutation can effectively eliminate the glycosylation at the specific site.

The FVIII expression was examined in hemophilia A (HA) mice carrying those plasmids via hydrodynamic injection. One week post the injection, no difference was found in FVIII activities in mice injected with either wild type BDD FVIII or the other FVIII variant plasmids.

The specific activities in mice receiving different FVIII plasmids didn't show significant difference either. These results indicate that the mutation didn't induce any significant conformation changes in the protein structures to affect the FVIII functional activity and production in vivo.

The impact of N-glycosylation on the immunogenicity of FVIII. Next, the immune responses against different FVIII variant were monitored. Anti-FVIII IgG levels appeared at low levels in all groups by day 14 and then began to rise and reach peak levels around day 56. By that time, N1810Q and N2118Q groups had lower IgG level compared to the other three groups.

Subsequently, a second plasmid challenge was performed on day 86. While all other groups had a drastic increase in anti-FVIII IgG levels, N2118Q group remained at a low level.

The inhibitor titers detected by a Bethesda assay parallels the anti-FVIII IgG levels. Low inhibitor titers were detected in the N1810Q and N2118Q groups after first plasmid transfer. After the second challenge, only the N2118Q group had lower inhibitor titer. Taken together, the results confirmed that when glycosylation at the 2118 site was eliminated, the immune responses decreased compared to WT-BDD FVIII and the other FVIII variants.

The impact of N-glycosylation on the immunogenicity of FVIII following AAV-mediated gene delivery. To confirm that N2118Q FVIII variant had lower immunogenicity, a different gene delivery method was used, particularly an adeno-associated virus (AAV) system.

HA mice were injected with AAV-BDD-FVIII and AAV-N2118Q-FVIII at dosage of 1012 vg/mouse. The FVIII activities were detectable one week post the injection and gradually increased to an average of 70% on week 4.

In AAV BDD-FVII treated mice, the activities dropped to lower levels on week 8 and continued to decline to less than 10% or undetectable levels on week 12.

In AAV-N2118Q-FVIII treated mice, the FVIII activities remained at a similar level on week 8 but slightly decrease on week 12. These results indicate the low titers of inhibitor developed in AAV-BDD-FVIII treated mice. On week 15, the FVIII activities reverted to higher levels suggesting potential tolerance induction between week 12 and 15 in those AAV injected mice.

Next, those mice were challenged with FVIII on a weekly basis for six weeks. One week after the final injection, inhibitor titers were detected. The AAV-BDD-FVIII treated mice had higher prevalence of inhibitor formation than AAV-N2118Q treated mice, indicating that mice treated with AAV-N2118Q-FVIII were more tolerant to FVIII infusion.

Identification of a potential T cell glycopeptide epitope surrounding site N2118 of FVIII. Since a reduced immune response was found in mice receiving N2118Q FVIII plasmid via hydrodynamic injection and AAV, the specific role of glycosylation at this site in interactions with the immune system was further investigated.

The 15-mer peptides around 2118 sites were used in in vitro proliferation experiments. The peptides were either without any glycosylation, with GlcNAc attached, or mannosylated at 2118 site. The splenocytes from HA mice with high titers of anti-FVIII IgG were isolated and cultured with those peptides, respectively.

The CD4+ T cell proliferation was detected, and multiple runs were performed and summarized. CD4+ T cells showed increased proliferation levels with 1 and 10 μM of MP1 while NGP1 and GlcP1 had negligible effects on cell proliferation. The results suggest that the MP1 had a T cell glycopeptide epitope compared to GlcP1 or NGP1.

The effect of mannosylation of FVIII on induction of T cell response. Next, to verify the effects of mannosylation of FVIII on T cell responses, mannan was used to block mannose receptor and observe how it affected T cells reacted with FVIII and MP1.

It was found that T cell proliferation was only partially inhibited in response to 0.1 U/ml FVIII. However, mannan inhibited T cell proliferation in response to MP1 at 0.1 and 1 μM. These data further confirmed that mannosylation at 2118 site is responsible for T cell activation.

Screening of glycopeptide epitope surrounding N2118 using overlapping peptides. To further identify the region of FVIII responsible for inducing an increased immune response against mannosylated peptide around 2118 site, the three mannosylated peptides and three non-glycosylated peptides were used. MP1 is 15-mer peptides with mannosylated at 2118 site in the middle of peptide. MP2 and MP3 are 15-mer peptides but shift either toward C or N terminus. The three non-glycosylated peptides, NGP1, NGP2 and NGP3, have the same amino acid sequence compared to their mannosylated counterparts.

Enhanced proliferation was observed when cells were cultured with MP1 and MP2 with dose dependence but nearly no proliferation was observed when cells were cultured with their non-glycosylated counterparts, NGP1 and NGP2. Cells cultured with MP2 had higher proliferation compared to MP1. On the other hand, MP3 showed little to no proliferation in comparison to the other mannosylated peptides and its non-glycosylated counterpart 2118NGP3. This data indicates that the essential sequence of the potential T-glycopeptide epitope region is located in the overlapping region of 2118MP1 and 2118MP2.

Conclusion. In conclusion, no significant changes were found in FVIII activities after removing each of the four glycosylation sites on FVIII in mouse model. The findings provide the opportunities to focus on investigating the impact of glycosylation on FVIII immunogenicity.

After plasmid transfer via hydrodynamic injection, mice carrying N1810 and N2118Q FVIII plasmids had lower immune responses. The mice receiving N2118Q FVIII plasmid had lower anti-FVIII IgG and inhibitor titers even after second plasmid challenge.

The reduction in immune responses of N2118Q was also observed in AAV experiments.

Reduced immune responses were detected in N2118Q FVIII variant, the specific roles of glycosylation were evaluated. Mannosylated peptides around 2118 are more immunogenic than GlcNc or non-glycosylated peptides.

In addition, the immunogenic region was narrowed down around the 2118 site. The potent T-cell specific glycopeptide epitope is located between K2111 and G2128 of FVIII.

These results can enhance the understanding of inhibitory antibody formation against FVIII and facilitate the development of a more tolerant FVIII molecule for replacement protein and gene therapy for hemophilia treatment.

### Example 3. Ultrasound Mediated Gene Delivery Specifically Targets Liver Sinusoidal Endothelial Cells for Sustained FVIII Expression in Hemophilia A Mice.

Abstract. Hemophilia A (HA) is a bleeding disorder in which an individual cannot produce functional clotting factor VIII (FVIII). The ability to target the native production site of factor VIII (FVIII)-liver sinusoidal endothelial cells (LSECs)—can improve the outcome of hemophilia A (HA) gene therapy. LSECs are a prime target for gene therapy because even minimal increases in FVIII functional activity can improve the quality of life. LSECs can be specifically targeted with ultrasound-mediated gene delivery (UMGD) at a lower power than those used to target hepatocytes in mice. In this example, energy conditions were explored that target LSECs with UMGD to induce persistent FVIII expression in HA mice. By testing a matrix of ultrasound-mediated gene delivery (UMGD) parameters for delivering a GFP plasmid into the livers of HA mice, specific conditions for targeted gene delivery to different cell types in the liver were determined. The mouse liver was injected via the portal vein with a combination of plasmid DNA and RN18 microbubbles (MBs). Simultaneously, to enhance gene transfer via cavitation, a pulsed therapeutic ultrasound (US) transducer was applied to the surface of the liver for one minute at 1.1 MHz frequency and 14 Hz pulse repetition frequency (PRF). HA mice were separated into two groups treated with different US conditions, low energy (LE; 50 W/cm2, 150 us PD) targeting predominantly endothelial cells or high energy (HE; 110 W/cm2, 150 us PD) targeting predominantly hepatocytes. This was coupled with a new endothelial-specific, high-expressing hFVIII plasmid, pUCOE-ICAM2-hF8/N6-X10. The activated partial thromboplastin time (APTT) assay was utilized to study hFVIII functional activity levels in the mouse plasma and the Bethesda assay for the formation of anti-FVIII inhibitors over 84 days. Livers were harvested at various time points, sectioned at 7 μm, stained, and imaged with a Leica DM6000 fluorescent microscope.

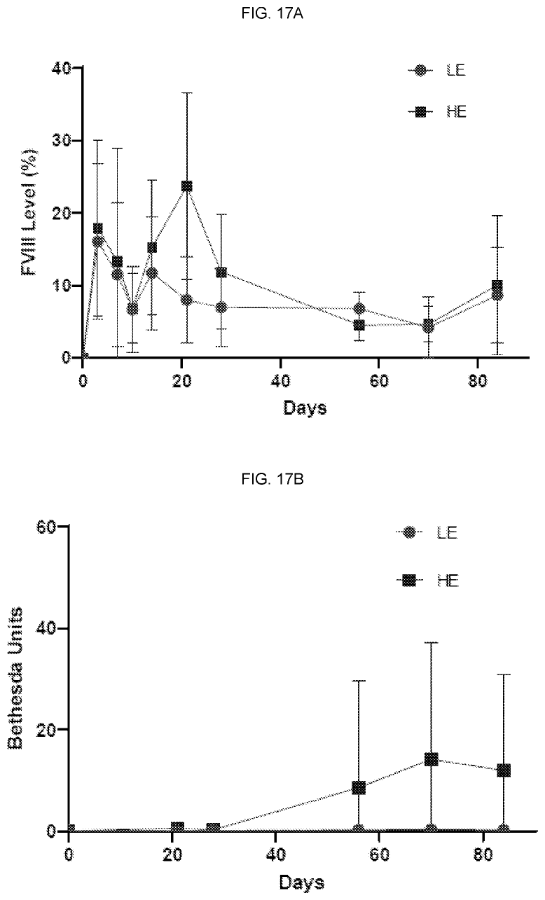

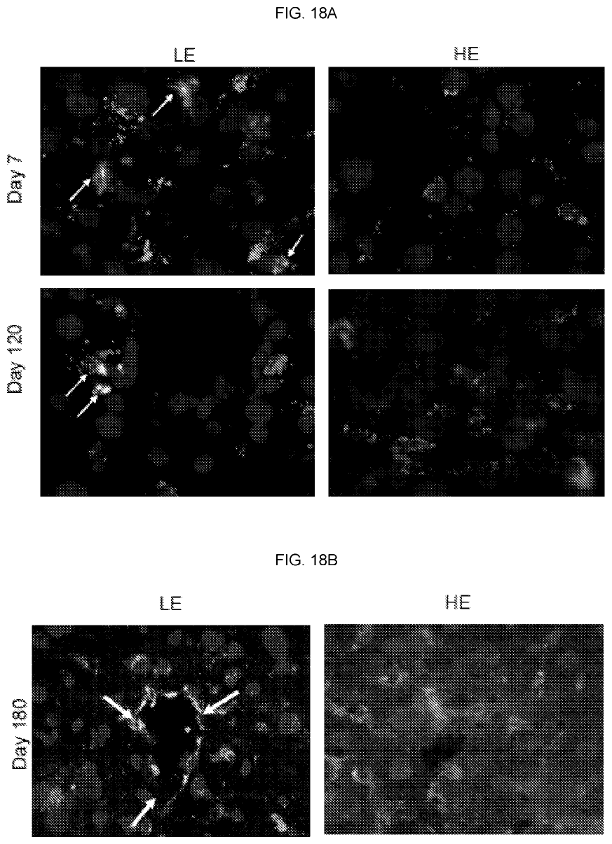

FVIII functional activity levels for the LE group were comparable to that of the HE group and stabilized at 10% at 84 days post-treatment, half of the HE-treated mice developed low-titer inhibitors, while none of the LE mice did (FIG. 17A). When assessing damage to the liver, alanine transaminase (ALT) levels were transiently increased in the initial few days post-treatment and rapidly returned back to the normal range in both the HE and LE groups. However, the LE mice showed a significantly smaller initial increase, indicating reduced transient liver damage compared to the HE group. Half of the HE-treated mice developed low-titer inhibitors over 84 days, while none of the LE mice had a measurable formation of inhibitors (FIG. 17B). RNAscope©® Multiplex Fluorescent staining showed occurrences of colocalization of hFVIII and Lyve-1 (an LSEC marker) mRNA at D7 and D120 in the LE mice. This indicates that the LE US conditions can efficiently deliver the endothelial-specific hFVIII plasmid to the LSECs to produce persistent, therapeutic levels of FVIII gene expression with reduced transient liver damage and inhibitor formation.

Introduction. Hemophilia A (HA) is an X-linked recessive genetic disorder that results in impaired production of factor VIII (FVIII) protein. FVIII participates in the intrinsic pathway of the coagulation cascade, and when its level is reduced or diminished, stable clots cannot be efficiently formed, leading to a HA phenotype that ranges from mild to severe. The current methods of treatment for HA involve repeated injections of FVIII to treat episodic bleeds or for prophylactic purposes. However, because of the relatively short half-life of FVIII, these repeated injections can be both costly and disruptive to the patient's everyday life (Chen, S L (2016). Am J Manag Care 22: s126-133.; Mannucci, P M (2020). Haematologica 105:545-553.). While advancements have been made to address the half-life of FVIII (Konkle, B A, et al. (2020). New Engl J Med 383:1018-1027.) as well as the formation of anti-FVIII inhibitors with the use of drugs such as emicizumab (Zhou, Z Y, et al. (2020). J Manag Care Spec Pharm 26:1109-1120.), frequent injections are still needed. Gene therapy as an alternative method of treatment for HA has garnered attention because it could provide a long-term solution to HA. In addition, small increases in FVIII production, as low as 1%, can have a marked improvement of the bleeding phenotype for severe patients. AAV-based gene delivery has shown promising results in HA gene therapy trials (George, L A, et al. (2021). N Engl J Med 385:1961-1973.), however, significant obstacles for those who develop high levels of anti-AAV antibodies demonstrate a need for a non-viral method of gene delivery (Hermans, C (2022). Orphanet J Rare Dis 17:154).

Ultrasound (US) mediated gene delivery (UMGD) in combination with microbubbles (MBs) has been shown to be an effective method for non-viral gene delivery. UMGD is an especially promising strategy for treating genetic diseases, including HA and other diseases with a basis in the liver. Microbubbles can briefly increase the membrane permeability of nearby cells through ultrasound-induced cavitation (Hernot, S, and Klibanov, A L (2008). Adv Drug Deliv Rev 60:1153-1166). This allows co-injected substances such as drugs or genetic material to have increased cellular uptake where the ultrasound is applied and has been tested successfully in vivo in a variety of organs (Chen, H H, et al. (2016). Expert Opin Biol Ther 16:815-826; Huang, Q, et al. (2012). Exp Neurol 233:350-356; Wan, C, et al. (2015). Mol Med Rep 12:4803-4814). In previous studies, the feasibility of delivering plasmid DNA (pDNA) into the liver of both HA and non-HA animals through UMGD has been established with optimization experiments in microbubble formulation (Sun, R R, et al. (2014). J Control Release 182:111-120), US parameter modifications (Shen, Z P, et al. (2008). Gene Ther 15:1147-1155; Tran, D M, et al. (2018). J Control Release 279:345-354), route of MB/pDNA delivery (Shen, Z P, et al. (2008). Gene Ther 15:1147-1155), and animal model (Manson, M A, et al. (2022). Blood Adv 6:3557-3568; Noble-Vranish, M L, et al. (2018). Mol Ther Methods Clin Dev 10:179-188; Song, S, et al. (2012). Mol Pharm 9:2187-2196). In one study (Song, S, et al. (2022). Mol Ther Nucleic Acids 27:916-926), a human FVIII (hFVIII) plasmid driven by a hepatocyte-specific promoter/enhancer (pHP-hF8/N6) was delivered via UMGD into HA mouse livers, attempting to target hepatic cells. While most of the animals had sustained FVIII gene expression of 8-20% up to day 60, a few individuals dropped to 0% FVIII activity with the concurrent formation of anti-FVIII inhibitors. These results demonstrated the possibility of long-term expression of an FVIII plasmid delivered via UMGD into mice, however, difficulties with immunogenicity remained.

This experimental example describes targeting the native production site of FVIII, liver sinusoidal endothelial cells (LSECs), as opposed to hepatocytes. FVIII produced in LSECs has the benefit of complexing immediately with Von Willebrand factor, which preserves its stability and function in the blood. In addition to this, the US power needed to target LSECs is predicted to be lower than that of hepatocytes because no endothelial membrane destruction is required to force pDNA and MBs to the extravascular space. This lower power can be less damaging to the liver, as well as less inflammatory to the immune system, resulting in a safe and novel application of UMGD for LSEC targeting. By utilizing a lower power of US wave and pulse duration combination, coupled with an endothelial-specific hFVIII plasmid, pUCOE-ICAM2-hF8N6X10, LSECs were specifically targeted and therapeutic treatment in HA mice was achieved using UMGD.

Materials and Methods. Animals. All procedures were performed according to the guidelines for animal care of Seattle Children's Research Institute (SCRI) as well as the National Institutes of Health. Protocols were approved by the Institutional Animal Care and Use Committees of SCRI. Eight-to sixteen-week-old FVIII exon 16 knockout HA/Bl6 mice, bred and maintained in a specific-pathogen-free (SPF) vivarium within SCRI, were used for the studies.

Plasmid and MB preparation. The hepatocyte-specific FVIII plasmid construct, pHP-hF8-X10 (Manson, M A, et al. (2022). Blood Adv 6:3557-3568), driven by a hepatocyte-specific promoter/enhancer (HP; composed of hepatic control region (HCR) and α1-antitrypsin promoter (hAAT)) was made by replacing the B-domain deleted (BDD)-hF8/N6 cDNA in the pHP-hF8/N6 plasmid (Cowan, P J, et al. (1998). J Biol Chem 273:11737-11744; Miao, C H (2005). Adv Genet 54:143-177) with a BDD-hF8-X10 variant cDNA (Cao, W, et al. (2020). Mol Ther Methods Clin Dev 19:486-495). The endothelial-specific FVIII plasmids, plCAM2-hF8N6-X10 and pUCOE-ICAM2-hF8N6-X10 were generated by replacing the HP promoter/enhancer in pHP-hF8-X10 with endothelial-specific ICAM2 promoter or UCOE-ICAM2 enhancer/promoter combinations and hF8-X10 cDNA with hF8N6-X10 cDNA. GFP reporter plasmid (pCMV-GFP) was driven by a ubiquitous cytomegalovirus (CMV) promoter. Large preparation of the plasmids were produced by either Genscript (Piscataway, NJ) or Aldevron (Fargo, ND) using standard industry techniques. Preparation of RN18 microbubbles was previously described by Sun et. al. (Sun, R R, et al. (2014). J Control Release 182:111-120). Briefly, 2,2-distearoyl-sn-glycero-3-phosphocholine (DSPC), 1,2-distearoyl-sn-glycero-3-phosphate (DSPA), and N-(carbonylmethoxypolyethyleneglycol 5000-DSPE) are mixed at a molar ratio of 82:10:8 (Avanti Polar Lipids, Alabaster, AL). Lipids were then resolubilized, and gas exchange was performed to fill the headspace of each vial with octafluoropropane gas (American Gas Group, Toledo, OH). MB size and concentration were measured using the qNano instrument with a NP2000 membrane (Izon Science, Christchurch, NZ). The average MB concentration and size was 1×10¹⁰ MB/ML and 1 μM, respectively. Immediately prior to use, MBs were activated by 45 seconds of vigorous agitation using a Vialmix™ shaker (Lantheus Medical Imaging, N. Billerica, MA).

US transducers and systems. The ultrasound system, as previously described (Song, S, et al. (2012). Mol Pharm 9:2187-2196; Song, S, et al. (2011). Gene Ther 18:1006-1014) was composed of a pulse generator, and a high-power radio frequency amplifier (JJ & A Instruments) controlled by a laptop interface that output ultrasound waves through a custom-designed single element, 16 mm diameter, 1.1 MHz transducer (Sonic Concepts, Bothell, WA). This transducer was applied directly to the exposed mouse liver with sterile ultrasound gel as a coupling agent.

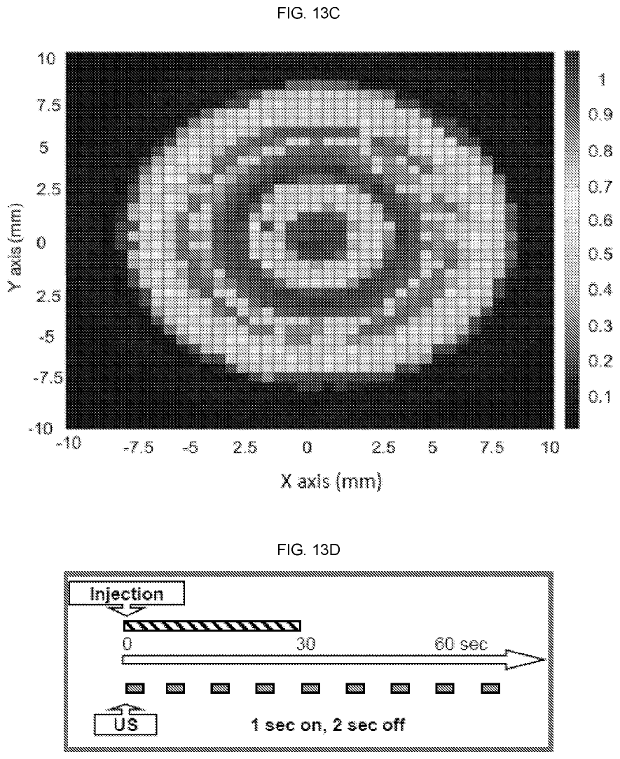

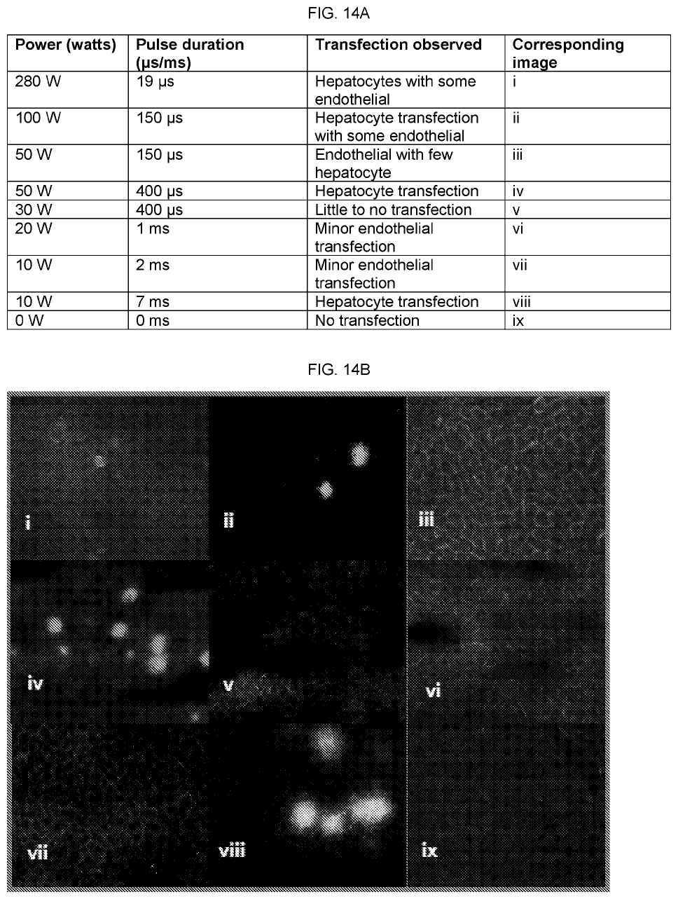

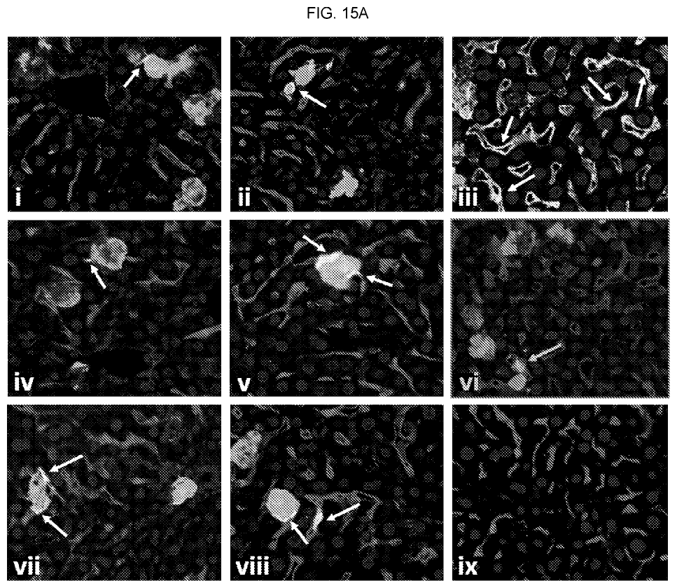

UMGD delivery of GFP reporter plasmid with matrix conditions. Mice were anesthetized by continuous inhalation of isoflurane during surgery and a midline incision was made to expose the liver and portal vein. A 400 μL mixture containing 2.5 μg per gram mouse of pCMV-GFP plasmid and RN18 MBs in PBS was injected into the liver via the portal vein for 30 seconds. Simultaneously to injection, the US transducer was applied to the liver surface, where the power and pulse duration settings were dependent upon experimental group and continued for 30 seconds after completion of injection for a total treatment time of 60 s (FIG. 13A). Hemostasis was applied to the portal vein until bleeding stopped. The muscle layer incision site was closed with sutures, the skin stapled together, and the mice are allowed to recover from anesthesia. Twenty-four hours post procedure, the livers were harvested and halved, with one half being placed in OCT and frozen at −80° C. and the other half getting fixed and stained as follows in the ‘Immunofluorescent staining’ methods. The frozen tissue was sectioned at 7 μm on a cryomicrotome (CM 3050S, Leica Co. Ltd, Deer Park, IL) for immunofluorescent staining, then imaged on a fluorescent microscope (DM6000B, Lecia, Deer Park, IL).

Hydrodynamic injection of plasmids. Mice were hydrodynamically injected with one of the three following plasmids at a concentration of 25 μg/mL and a volume equal to 9% the body weight of the mouse via the tail vein. Whole blood was collected on days 1 & 7 via retro-orbital collection to measure the level of FVIII activity. Livers were collected on day 7 and frozen in OCT at −80° C. for RNAscope or fixed for immunofluorescent staining.

The RNAscope® Fluorescent Multiplex Assay. Seven-micron sections of liver were produced on the Leica CM3050S Research Cryostat and mounted on slides. The slides were fixed in 10% neutral buffered saline for 1 hour then subjected to the RNAscope© Muliplex Fluorescent Reagent Kit v2 (ACD Bio, Newark, CA) according to protocols from ACD as follows. Sections were dehydrated sequentially in 50%, 70%, and 100% ethanol and stored in 100% ethanol overnight or up to 2 weeks. Slides are removed from the EtOH, hydrogen peroxide (ACD) was added for 10 minutes, then protease inhibitor IV (ACD) was added for 30 minutes at RT. A positive control probe (Mm-Ppib-Mus musculus peptidylprolyl isomerase B, a highly expressed protein in mouse tissue), negative control probe (binds dihydrodipicolinate reductase (dapB) protein found in bacteria such as E. coli), and custom-designed experimental probe, all from ACD, were incubated on slides for 2 hours at 40° C. Slides were then exposed to the amplification series, AMP 1-3 (ACD) at 40° C. This was followed by staining for each channel, which consisted of the sequential addition and incubation of HRP for that channel (1, 2, or 3), the fluorophore, and the HRP blocker. Slides were mounted with 4 drops of Fluoromount-GTM with DAPI and evaluated on a Leica DM6000 fluorescent microscope.

Immunofluorescent staining. Livers of mice that were given the pCMV-GFP plasmid were cryoprotected in 30% sucrose at 4° C. overnight following fixing in 4% paraformaldehyde (RT-15710, Electron Microscopy Sciences, PA) at room temperature 1 hour.

All mouse livers were cut to 7 μm thick with cryomicrotome (CM 3050S, Leica Co. Ltd, USA). Sections were prefixed in cold acetone −20° C. for 20 minutes and blocked in 0.5% fetal bovine serum in PBS (phosphate-buffered saline). One hour after blocking, the sections were incubated with primary antibodies against CD31 (14-0311-81, Thermo Scientific), LYVE-1 (AF2125, R&D Systems, MN), and GFP (A10262, Thermo Scientific, WA), or FVIII (SAF8C-AP, Affinity Biologicals, Ontario, Canada) then diluted in blocking buffer at 4° C. overnight. After three washes in PBS for 10 minutes, secondary antibodies conjugated with Alexa Fluor 488 or 594 (Invitrogen, CA, USA) diluted in blocking buffer were applied to section for 1 hour at room temperature. Sections were cover-slipped with mounting medium containing DAPI (H-1200-10, Vector Laboratories, CA, USA) for nuclear staining. Images were examined on a fluorescent microscope (DM6000B, Lecia).

Therapeutic UMGD of FVIII in HA mice. UMGD was performed as described in the section of UMGD delivery of GFP reporter plasmid above on HemA/B16 mice between 8 and 14 weeks of age. A pretreatment injection of recombinant hFVIII was given to avoid excessive bleeding of the hemophilic animals during surgery procedure. All of the mice received 2.5 μg per gram mouse of UCOE-ICAM-F8N6X10 plasmid, RN18 MBs, and hFVIII protein in PBS via the portal vein injection. Hemostasis was applied and gelfoam was placed on the injection site to aid in clot formation. These mice were followed for up to 180 days post-procedure.

Assessment of FVIII activity & anti-FVIII inhibitor antibodies. Whole blood was collected from mice in 3.8% sodium citrate solution via retro-orbital bleeding. Blood plasma was separated via centrifugation at 5000 rpm for 5 minutes. FVIII activity was measured via activated partial thromboplastin time (aPTT) using a modified clotting assay with FVIII-deficient plasma59. The anti-FVIII inhibitor concentration was measured using the Bethesda assay as previously detailed (Ye, P, et al. (2004). Mol Ther 10:117-126; Kasper, C K, et al. (1975). Thromb Diath Haemorrh 34:612).

Results. Screening ultrasound conditions that can target LSECs. To successfully target LSECs, previously established effective UMGD surgical methods were used while modifying the US conditions used (FIG. 13A). The transducer used in these experiments is H158; a single-element, unfocused transducer that generates a pressure profile and pressure map as shown in FIGS. 13B and 13C, respectively.

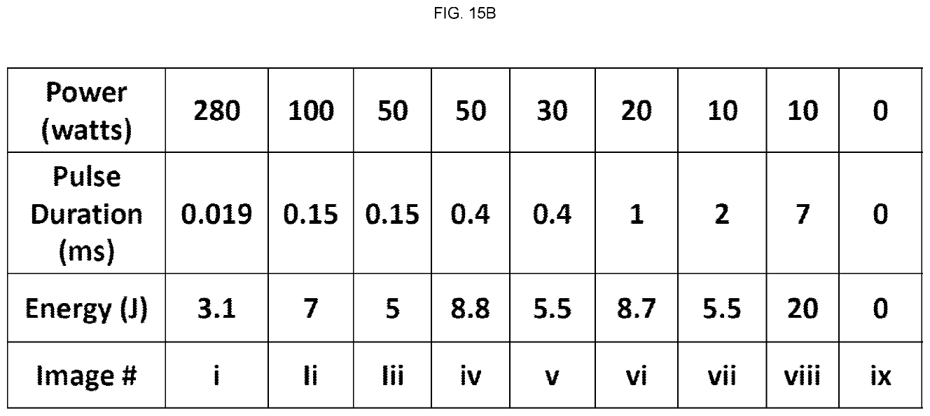

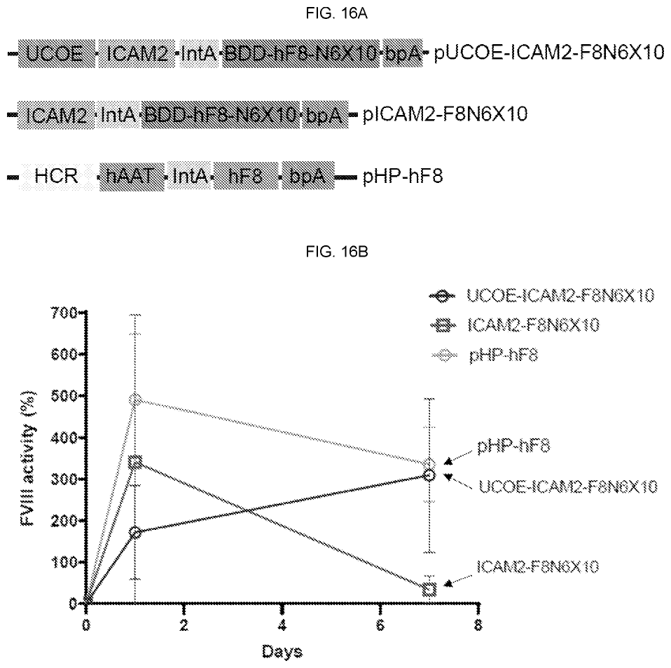

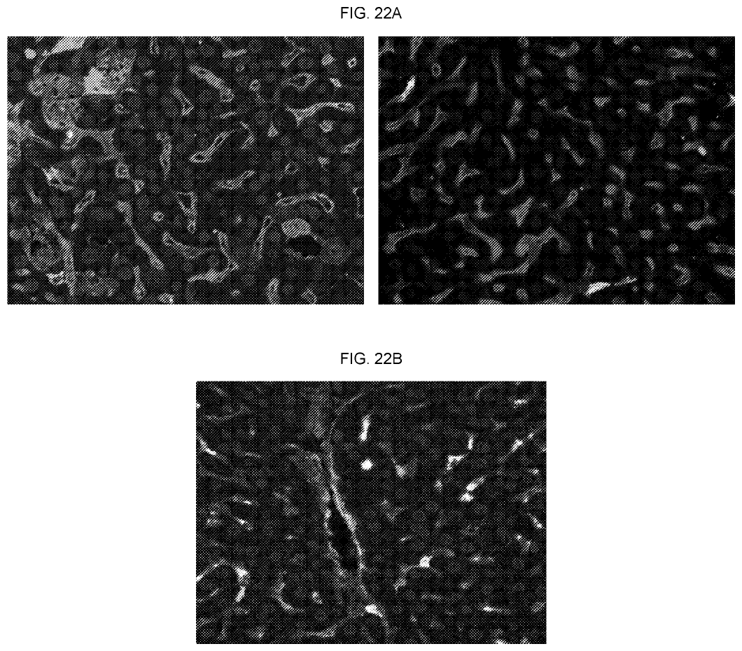

Previous gene transfer experiments targeting mainly hepatocytes (Tran, D M, et al. (2018). J Control Release 279:345-354) have highlighted the effect of altering the US parameter pairings-pulse duration and power, on the transfection level. For increasingly long pulse durations, the best gene expression was found at decreasing PNP values. It appeared the local maximums in each experimental pulse duration set clustered along a treatment energy curve between 7J to 9J energy curve. Thus, US parameters in the maxima or higher energy curve were chosen (7-9J; indicated as ii, iv, and vi in FIG. 15A) for hepatocyte-targeting and in a lower energy curve compared to the previous maxima. A matrix of US conditions where power ranges (5-5.5 J; indicated as iii, v, and vii in FIG. 15A) from 10 W-100 W and pulse duration (PD) from 150 μs-1 ms was created for LSEC-targeting. Two previous conditions with significantly shorter (19 μs and 280 W/cm²; indicated as i in FIG. 15A) and higher PDs (7 ms and 10 W/cm², indicated as viii in FIG. 15A) were also tested. Two BL6 mice per condition pairing were treated with UMGD using a GFP plasmid driven by ubiquitous cytomegalovirus (CMV) promoter (pCMV-GFP) and RN18 MB solution injected into the portal vein. This experiment was repeated once with separate groups of mice for a total of n=4 per condition. As a control, BL6 mice were injected with the same GFP plasmid and MB solution, without ultrasound treatment (indicated as ix in FIG. 15A). Twenty-four hours after UMGD, livers were harvested, sectioned to 7 μm, stained with GFP and an endothelial marker (Lyve-1), and imaged on a fluorescent microscope. FIG. 15B shows not only GFP cell transfection patterns in the liver but also regions of colocalization between the delivered GFP and the location of LSECs. The two previously used liver-targeting US conditions, 280 W/cm², 19 μs PD, 2.5 MPa PNP and 100 W/cm², 150 μs PD, 1.5 Mpa PNP US condition, displayed predominant transfection in hepatocytes with minor transfection in endothelial cells (indicated as i and ii in FIG. 15B). The 50 W/cm², 150 μs PD, 1.1 MPa peak negative pressure (PNP) condition showed the highest widespread endothelial-specific cell transfection (indicated as iii in FIG. 15B). Additionally, liver sections taken from the same mouse but at alternative depths and stained in the same way (FIG. 22A), showed minor hepatic as well as endothelial transfection on different slides. Based on these results, there is a location dependent effect on transfection within the liver due to the slight variation in US power at various depths. Despite this, the 50 W/cm², 150 μs PD showed the highest endothelial-specific transfection. Liver sections from the 50 W/cm², 150 μs PD mice were also stained with anti-CD-31 (alternative endothelial marker) and anti-GFP (FIG. 22B). Similarly, to the anti-Lyve stained sections, these sections had high levels of colocalization between GFP and CD-31. The other combinations showed transfection mainly in hepatocytes with minor/moderate transfection in endothelial cells (indicated as iv, v, vi, vii, and viii in FIG. 15B) and no GFP signal was detected in the untreated mouse liver (indicated as ix in FIG. 15B).

Selection of endothelial-specific high-expressing hFVIII plasmid. To target LSECs, an endothelial-specific human FVIII (hFVIII) variant plasmid (ICAM2-hF8X10/N6; FIG. 3A) under the control of an endothelial-specific human intercellular adhesion molecule 2 (ICAM2) promoter was designed (Cowan, P J, et al. (1998). J Biol Chem 273:11737-11744). In addition, a minimal ubiquitous chromatin opening element (UCOE) (Muller-Kuller, U, et al. (2015). Nucleic Acids Res 43:1577-1592) was incorporated into the endothelial-specific construct (UCOE-ICAM2-hF8X10/N6) to prevent transgene silencing and achieve consistent, stable, and high-level gene expression. The hF8/N6 gene encodes an FVIII B domain deletion variant that contains a N-terminal amino acid stretch with 6 glycosylation sites for enhanced secretion efficiency (Song, S, et al. (2022). Mol Ther Nucleic Acids 27:916-926; Miao, H Z, et al. (2004). Blood 103:3412-3419). In addition, the hF8X10 variant also includes 10 amino acid porcine FVIII-like substitutions in the A1 domain of the heavy chain to achieve higher levels of gene expression (Song, S, et al. (2022). Mol Ther Nucleic Acids 27:916-926; Cao, W, et al. (2020). Mol Ther Methods Clin Dev 19:486-495).

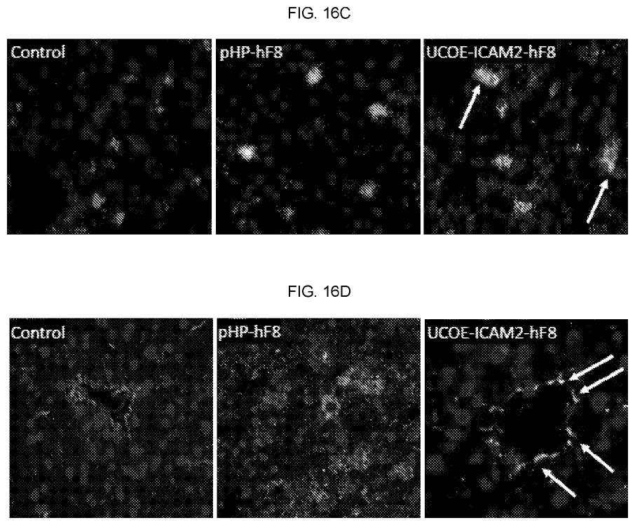

Three groups of HA/Bl6 mice were hydrodynamically injected with one of three plasmid constructs: pUCOE-ICAM2-F8N6X10, pICAM2-hF8N6X10, or pHP-hF8/N617, to compare the FVIII expression levels following gene delivery of these constructs. Blood plasma samples taken on day 1 and day 7 were measured for FVIII activity levels using the activated partial thromboplastin time (aPTT) assay (FIG. 16B). The average FVIII activity in the groups of mice given the pUCOE-ICAM2-F8N6X10 were 3-4 fold lower than that in PHP-hFVIII/N6 treated mice on day 1, however, both groups reached approximately 300% on day 7, with no significant difference between the two. The group of mice given the ICAM2-hF8N6X10 plasmid had a significantly lower FVIII activity percentage at approximately 40% on day 7.

Next, the target cells that showed FVIII gene expression in the treated mice wre examined with RNAscope and immunofluorescent imaging assays. Livers from both the pHP-hVIII/N6 and UCOE-ICAM2-hF8N6X10 injected mice were collected at day 7 and sectioned. For RNAscope imaging (FIG. 16C), which stains individual molecules of mRNA, the slides were incubated with probes that targeted hFVIII and Lyve-1 (a LSEC marker). Both groups of mice showed successful widespread distribution of hFVIII mRNA staining. However, only the mice injected with the endothelial-specific UCOE-ICAM2-hF8N6X10 plasmid, displayed evidence of colocalization between the hFVIII and Lyve-1. This demonstrates that this plasmid successfully generated specific FVIII expression in LSECs. Similar results were seen in the immunofluorescent images in both groups of mice (FIG. 16D). Here liver sections were stained using anti-hFVIII and anti-CD-31 (an endothelial marker) primary antibodies. hFVIII protein expression was visible in both groups of mice, however, only the UCOE-ICAM2-hF8N6X10 mice had evidence of colocalization between the hFVIII and CD-31 protein signals. This data reinforces the expectation that UCOE-ICAM2-hF8N6X10 will primarily target LSECs.

Targeting LSECs with UCOE-ICAM2-hF8N6X10 through UMGD. To compare the success of the different US condition pairings in targeting predominantly LSECs with an endothelial-specific hFVIII plasmid, HA mice were randomly separated into two experimental treatment groups. Mice in the low energy (LE) condition were treated with 50 W/cm² (PNP 1.1 MPa), 150 μs PD US, n=17, and mice in the high energy (HE) condition group 100 W/cm² (PNP 1.5 MPa), 150 μs PD US, n=14, for a total time of 60 seconds with direct placement of the US transducer on the surface of the liver. For the first 30 seconds of treatment, a solution of RN18 microbubbles and UCOE-ICAM2-hF8N6X10 plasmid were injected into the portal vein of the mouse. The experimental mice were allowed to recover and were followed over time.

To determine FVIII activity derived from the UCOE-ICAM2-hF8N6X10 plasmid following UMGD, blood was taken from HE & LE mice at multiple time points over 84 days and analyzed with the aPTT assay (FIG. 17A). Blood collection did not start until day 3 following the surgery to avoid measuring residual FVIII activity from the pre-treatment injection of FVIII protein which was given to aid in blood coagulation during open surgery of HA mice. The data showed no significant difference between the LE and HE FVIII activity at each timepoint, with activity ranging between 5-25%. Both groups of mice showed 10% FVIII activity at day 84.

HA mice are prone to develop anti-FVIII inhibitors following treatment with FVIII. To measure the formation of these inhibitors, plasma samples were tested with the Bethesda assay beginning at day 14 through day 84 in both groups of mice. FIG. 17B shows the average FVIII inhibitor formation during this time. Fifty percent of the HE mice had measurable levels of inhibitors after day 28 which initially increased before plateauing around day 70. None of the LE mice tested had any detectable inhibitors.

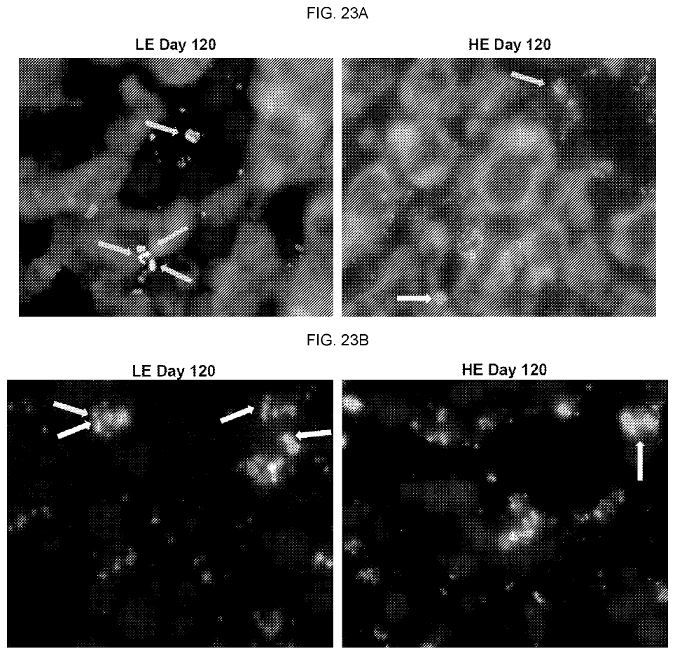

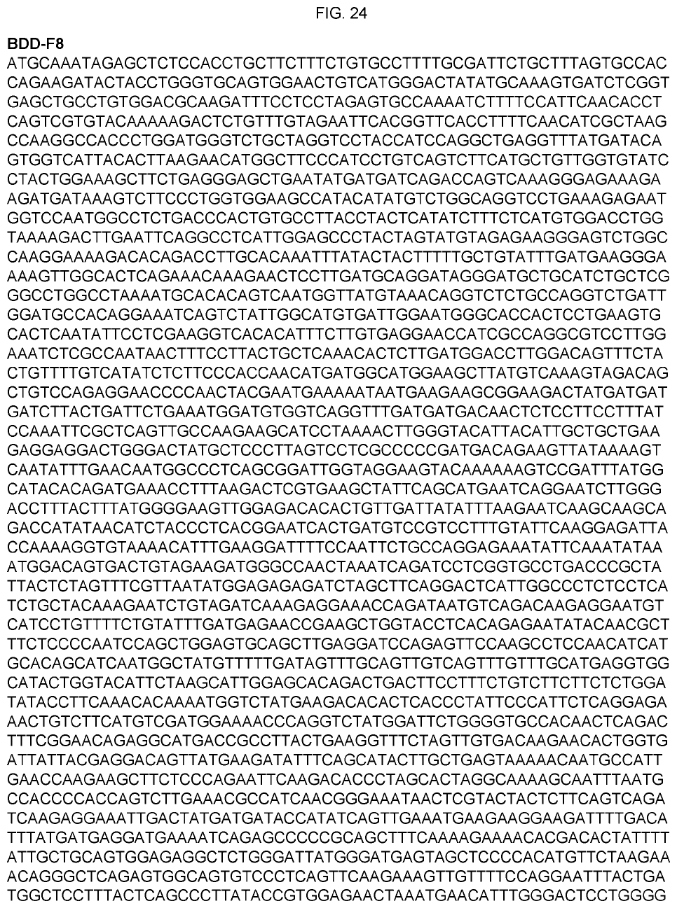

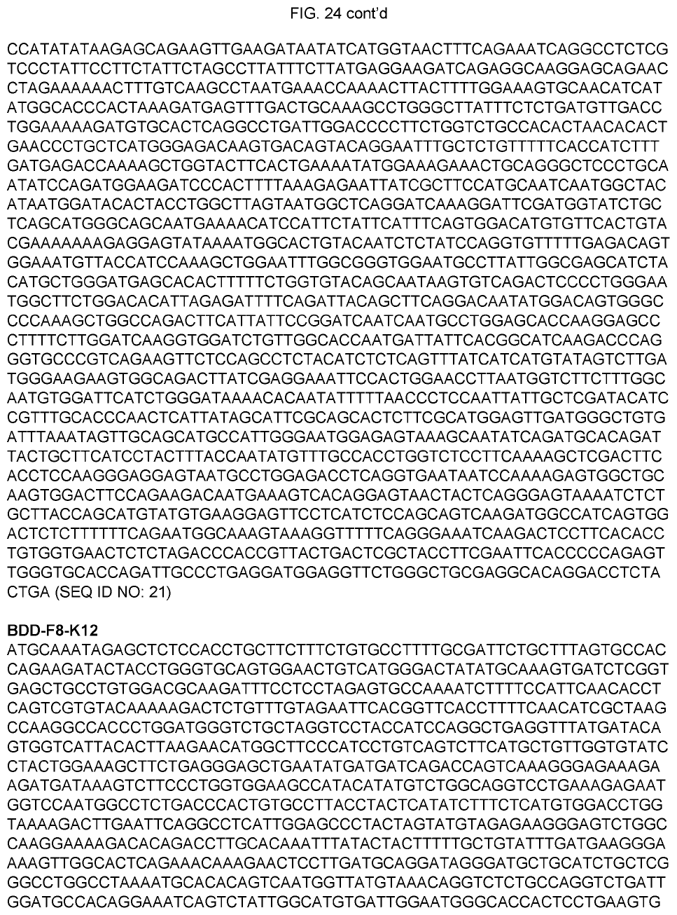

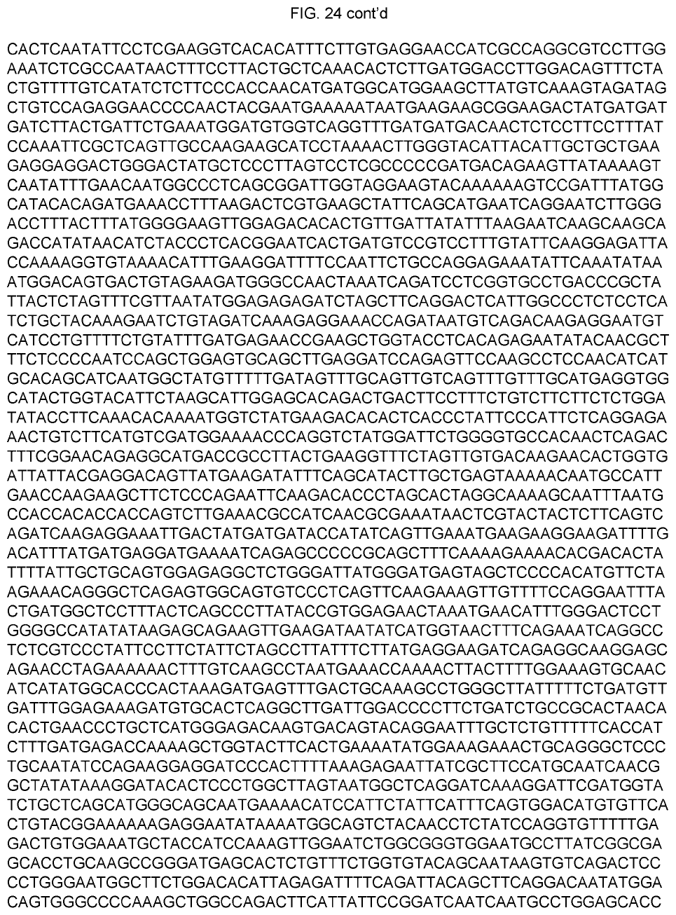

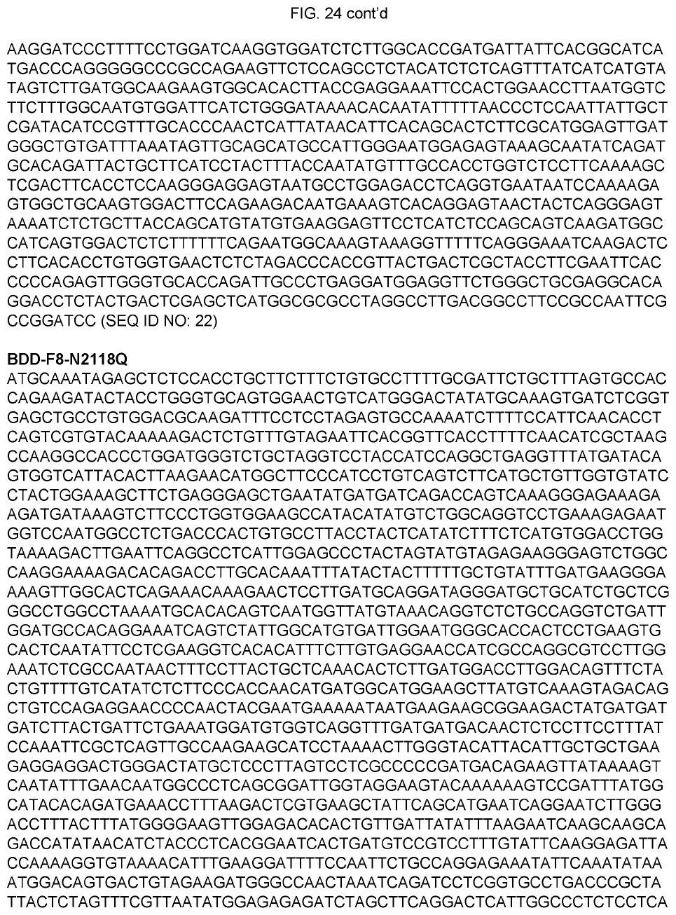

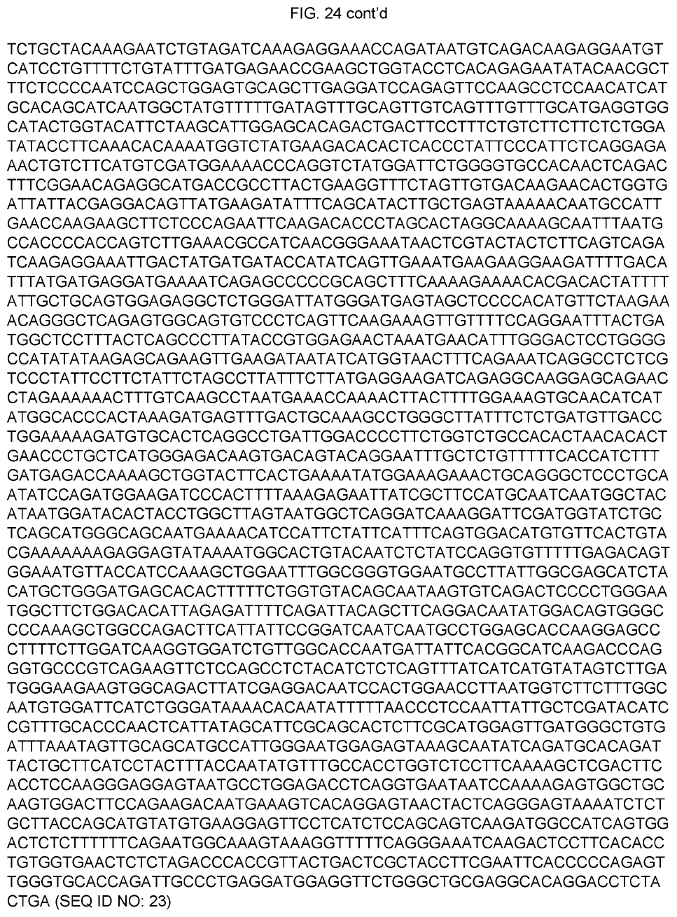

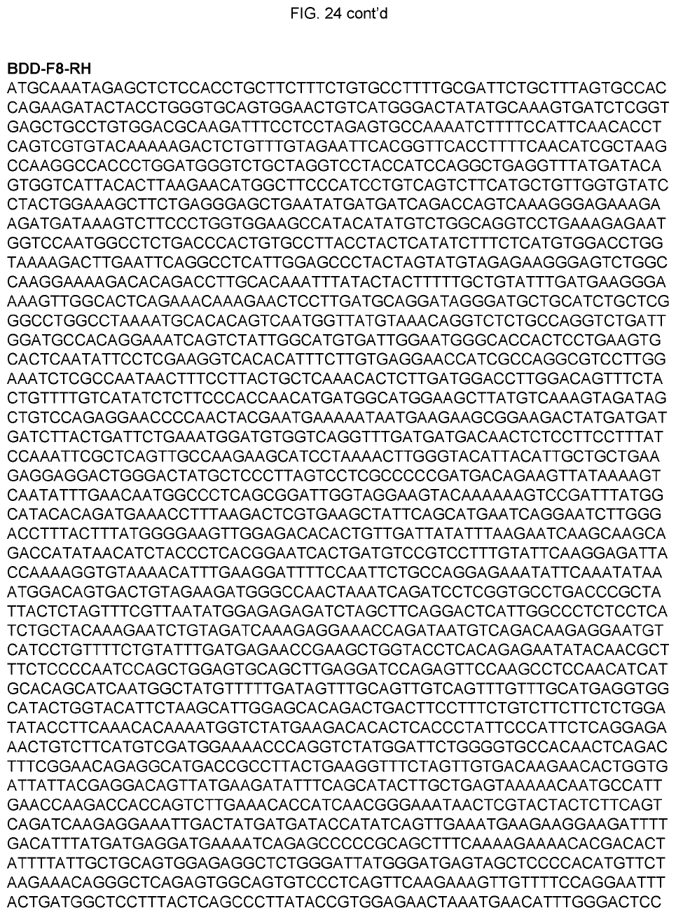

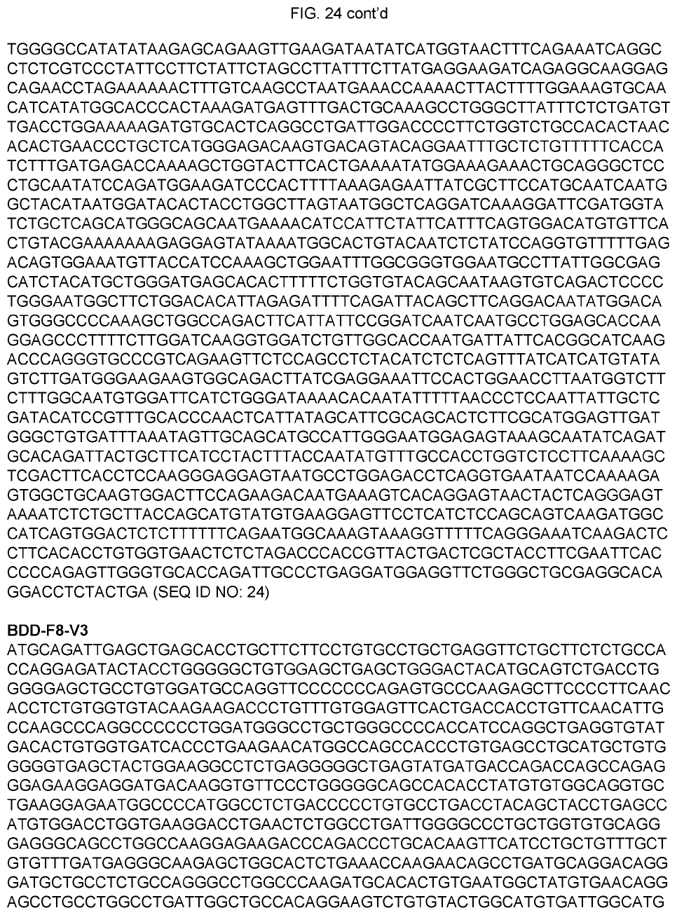

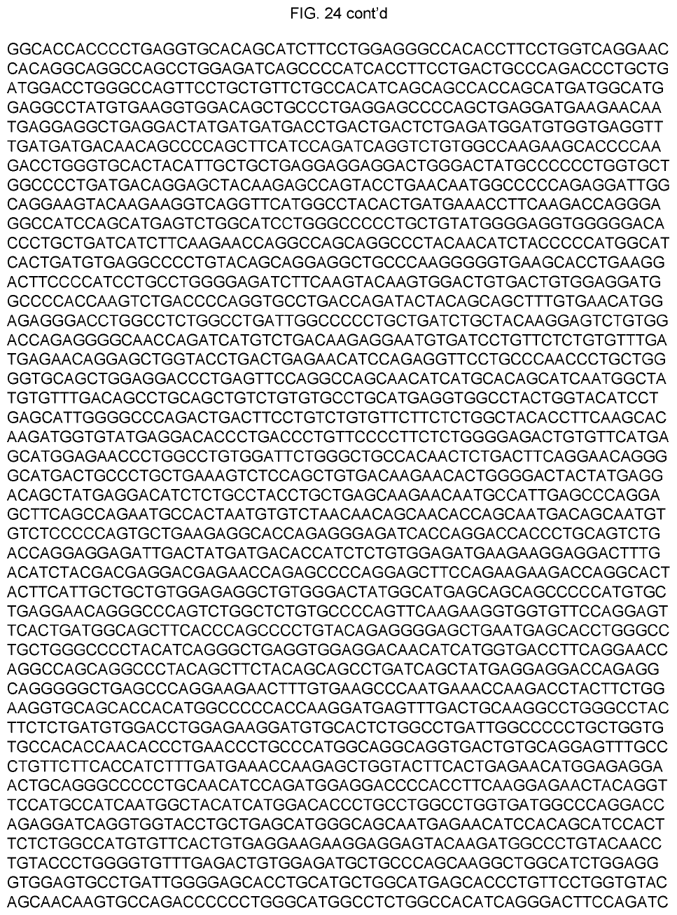

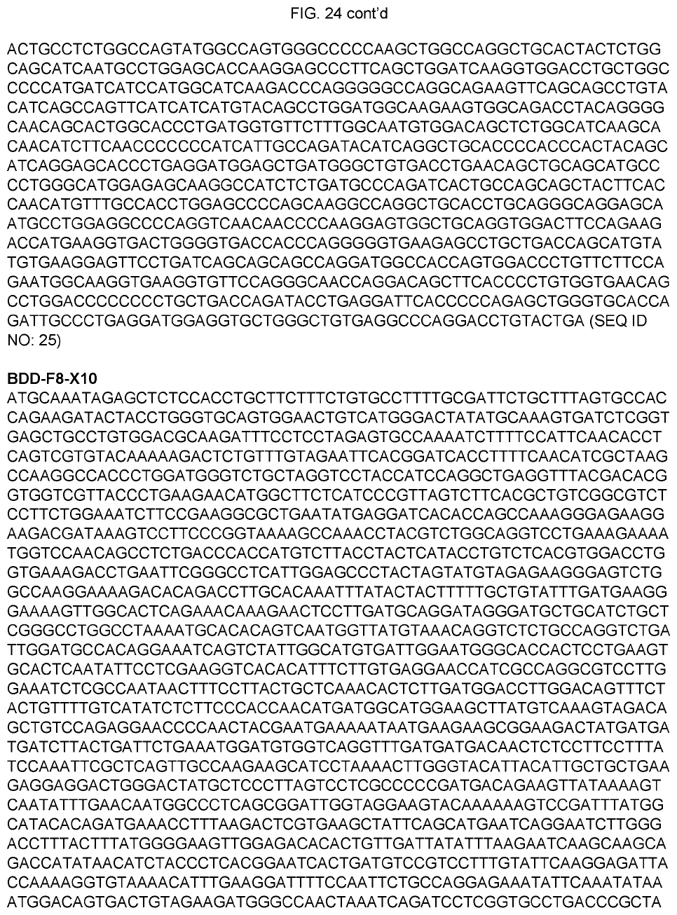

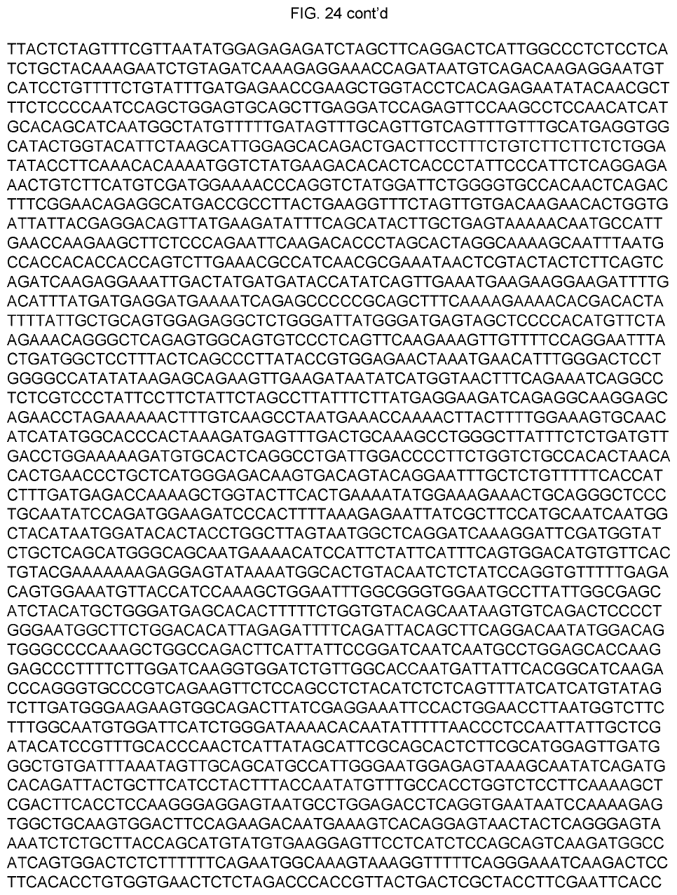

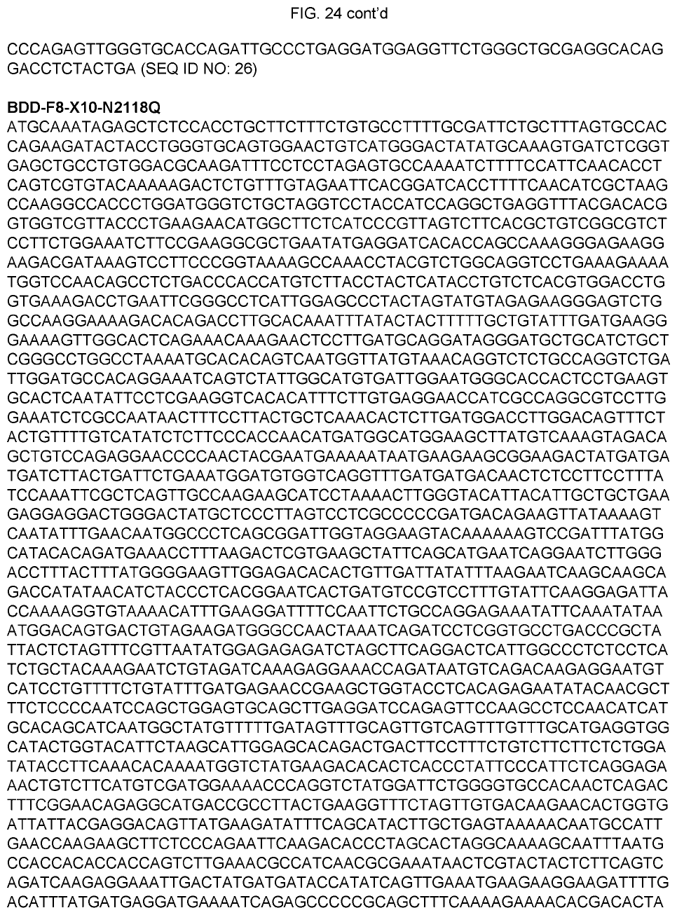

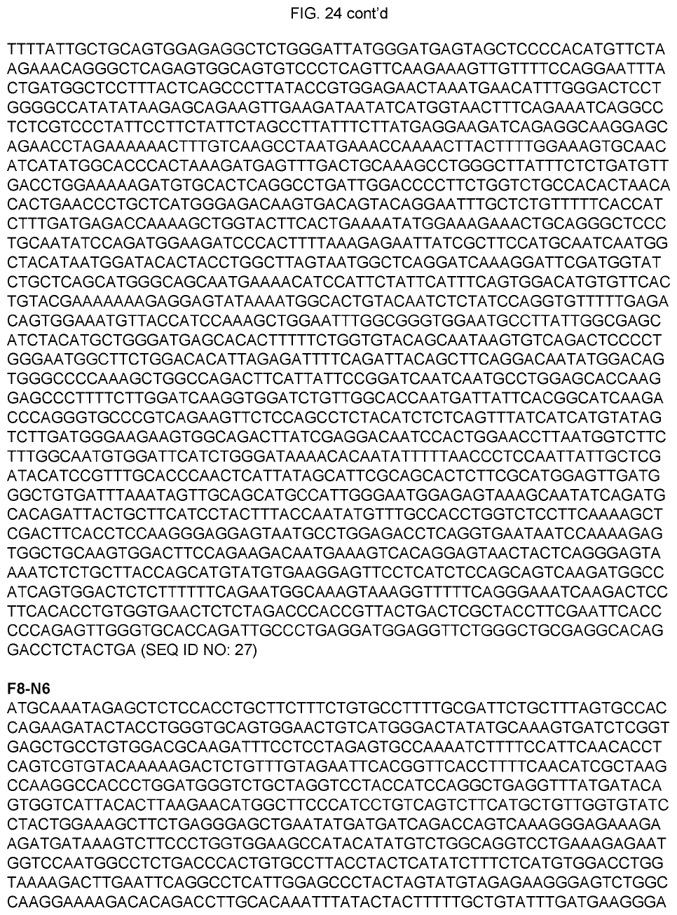

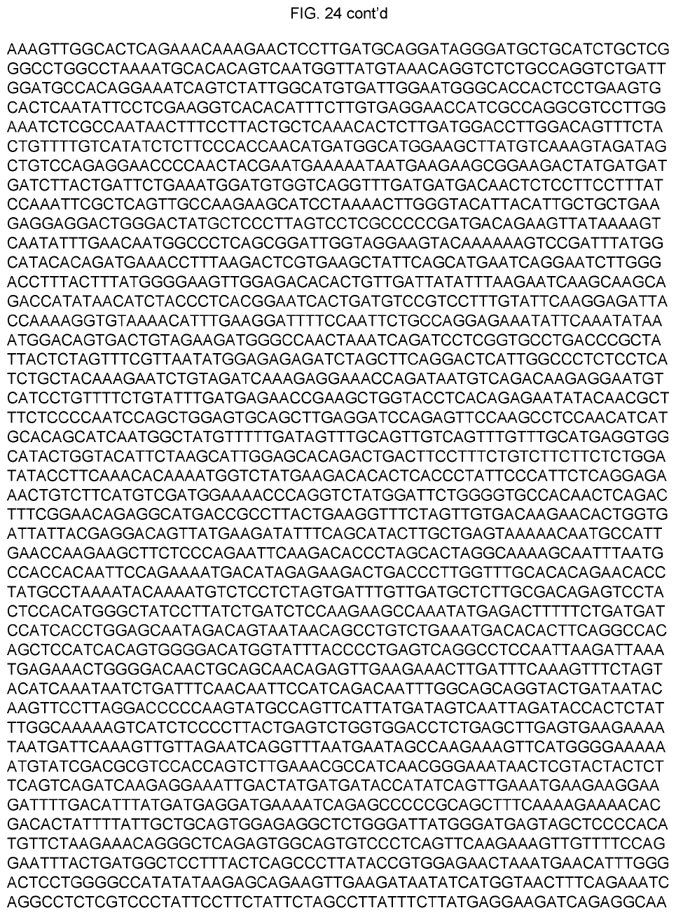

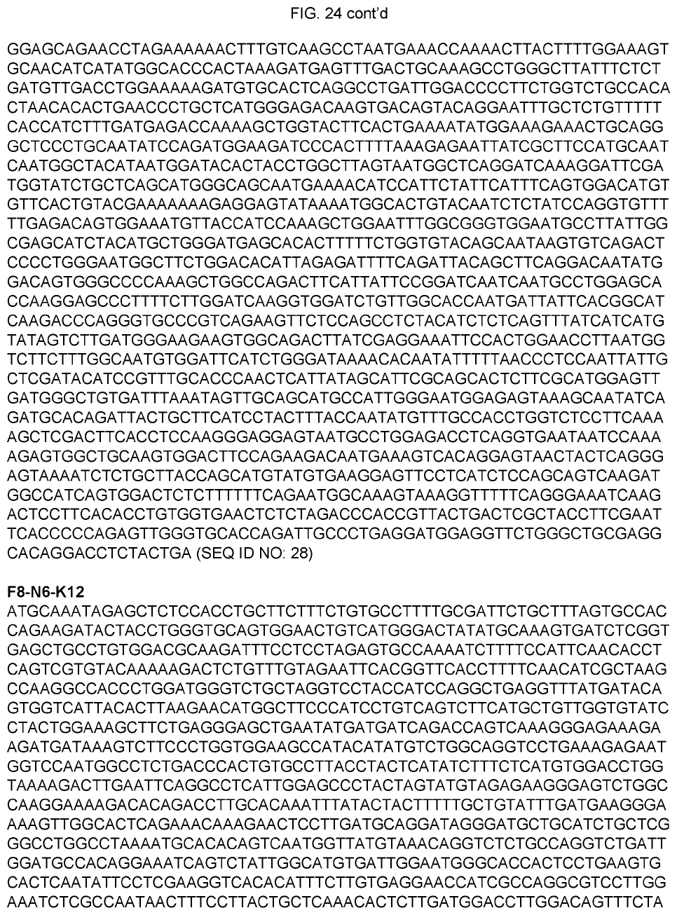

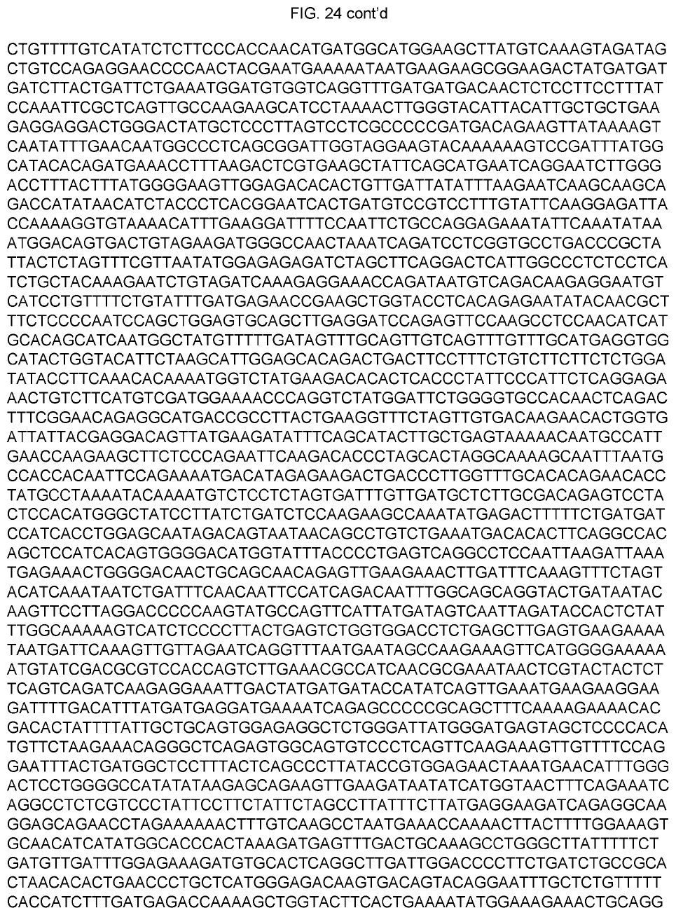

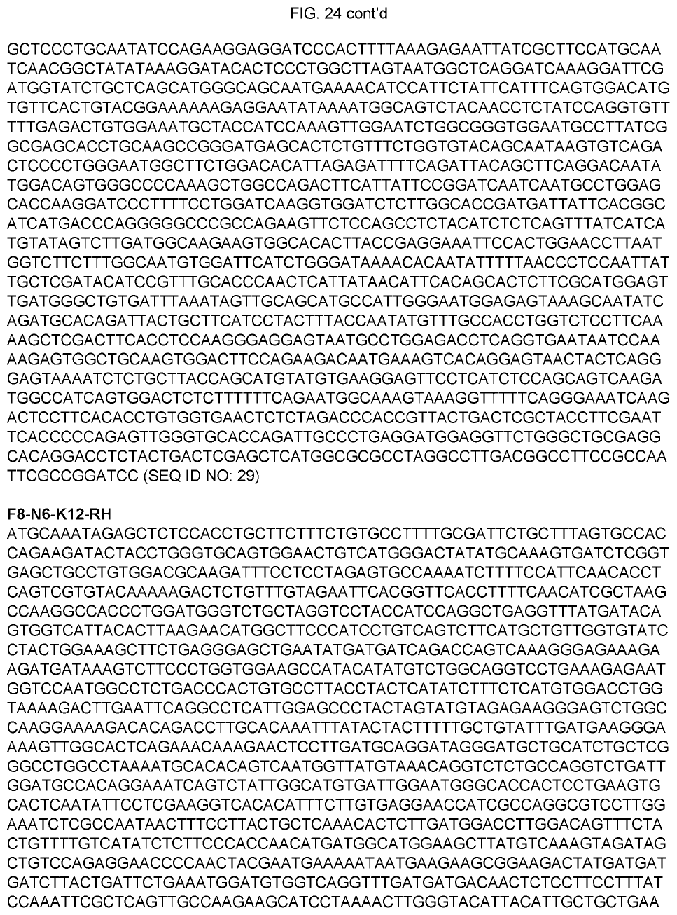

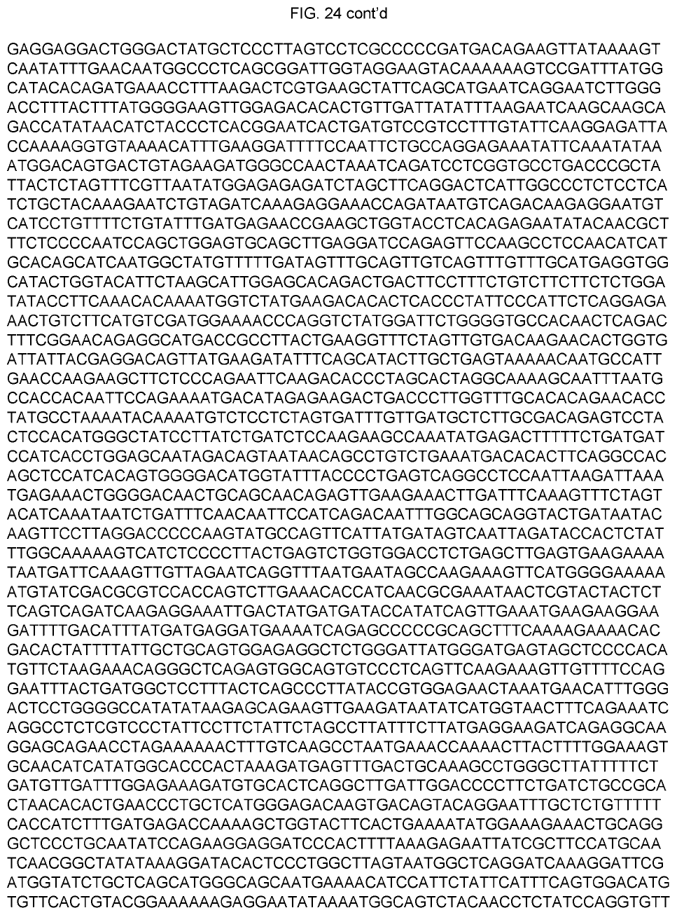

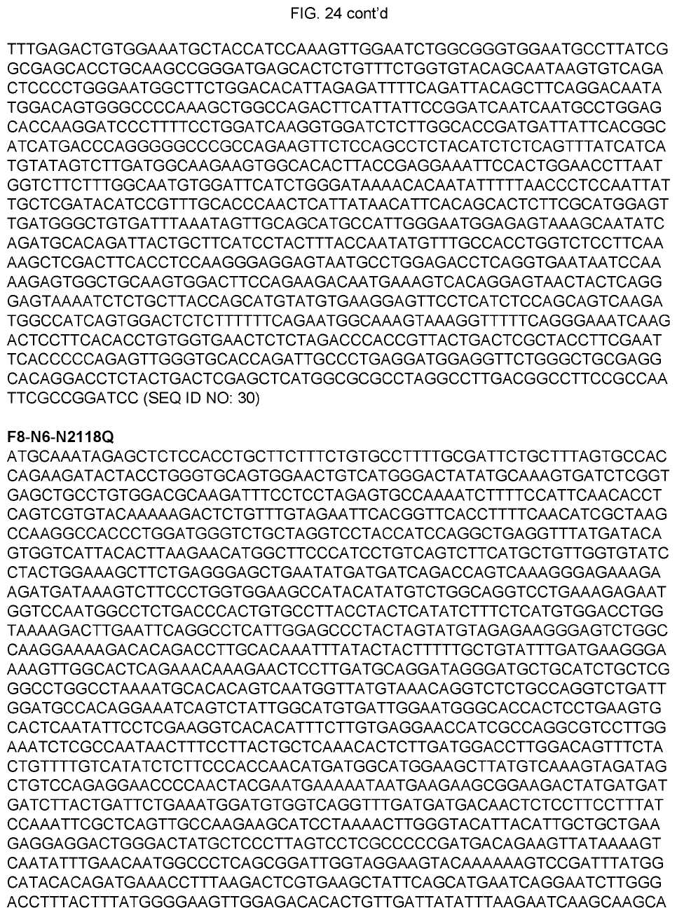

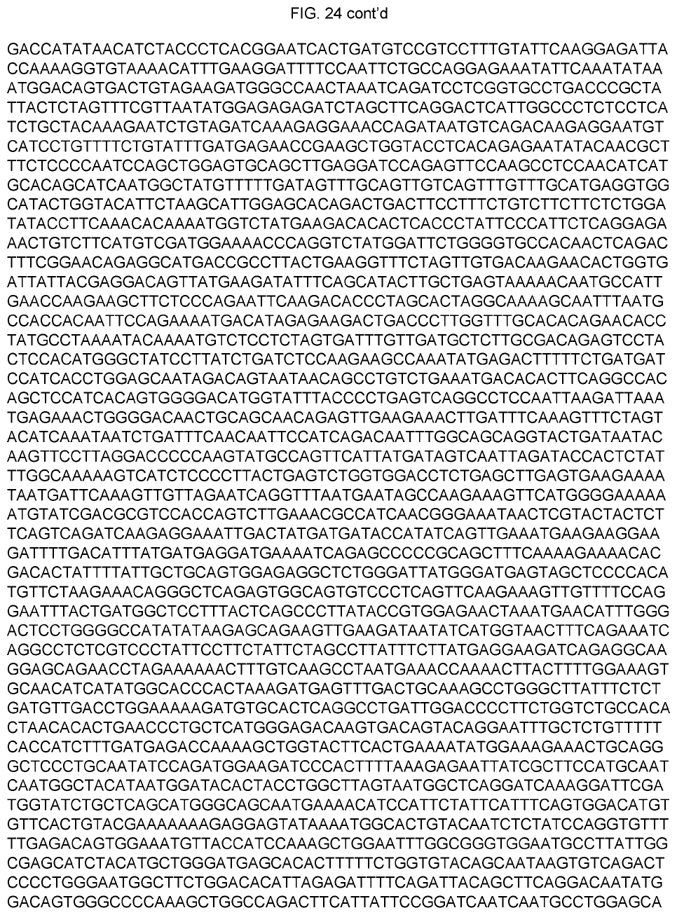

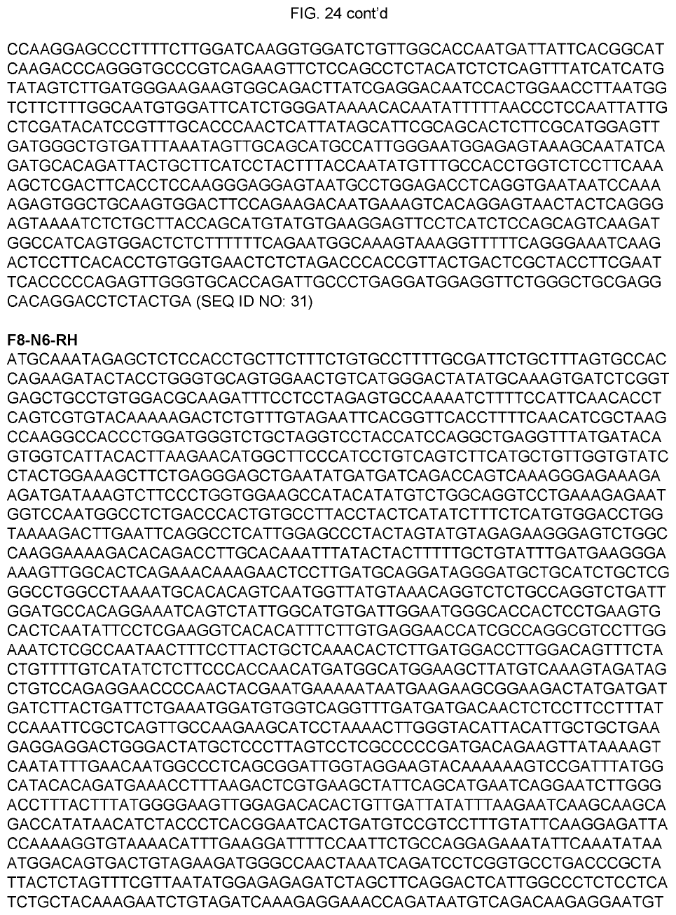

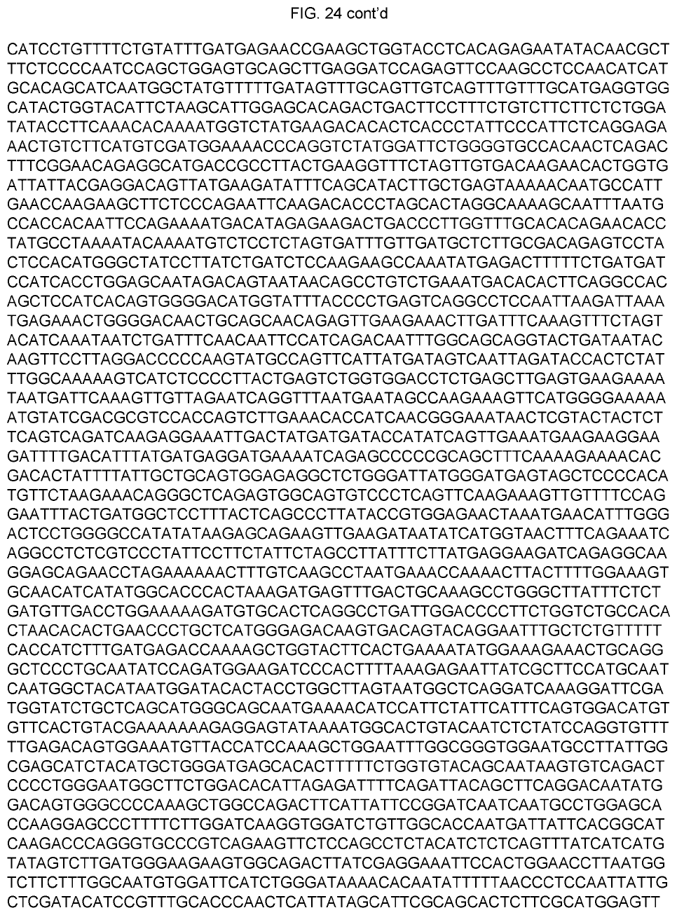

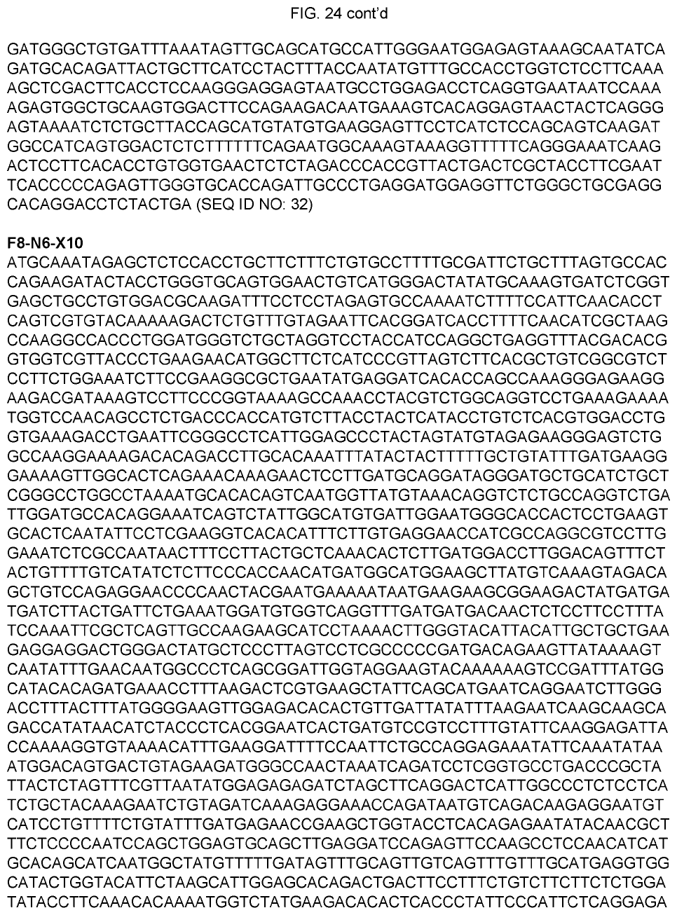

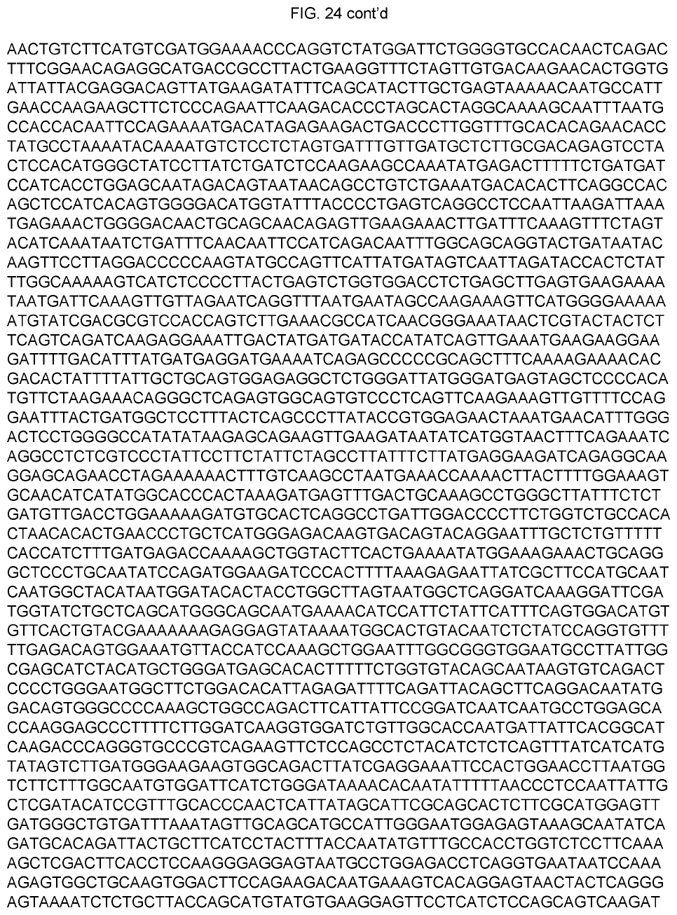

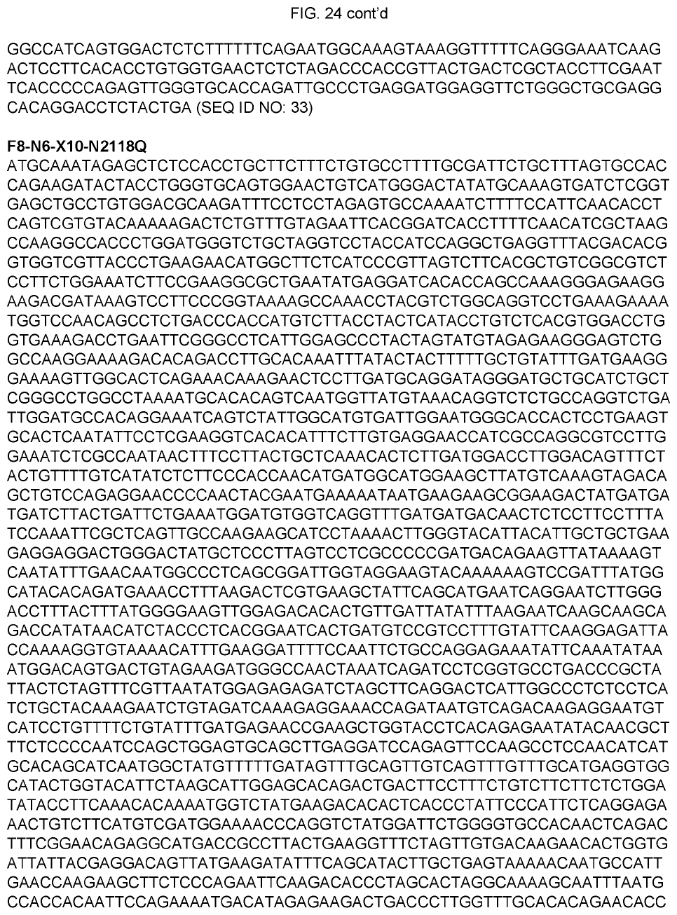

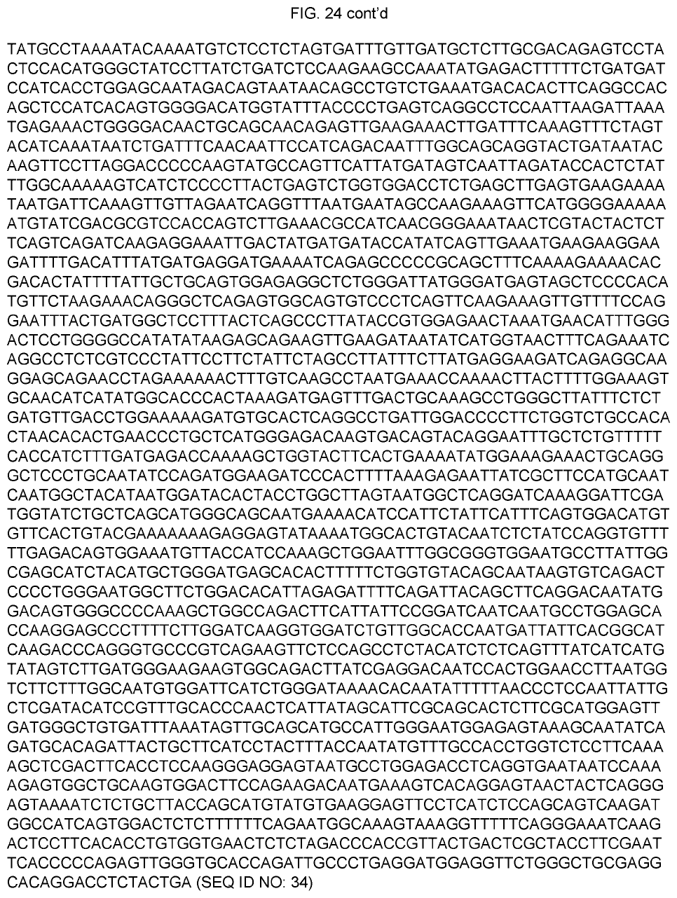

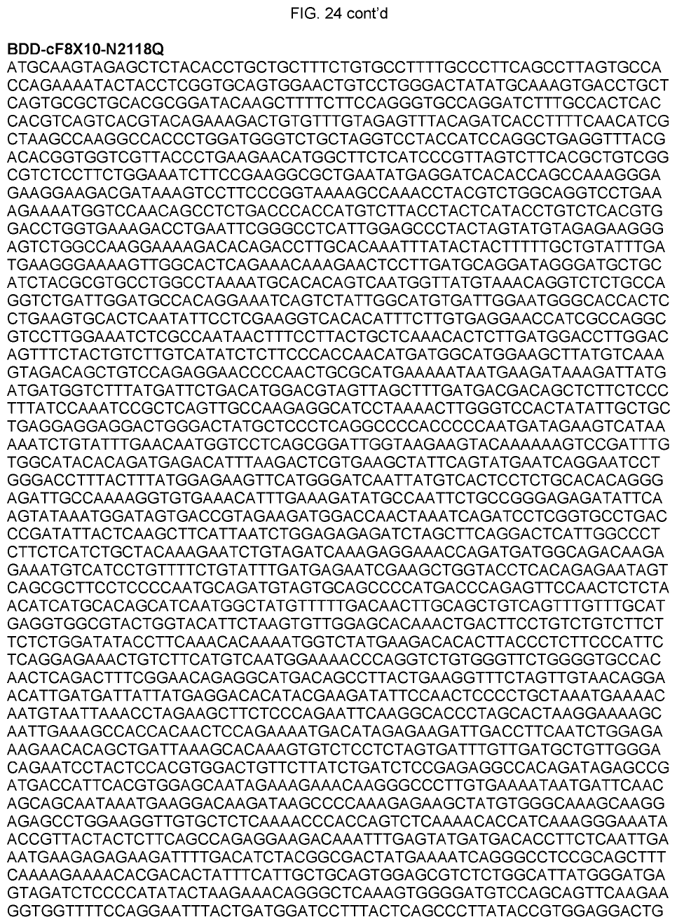

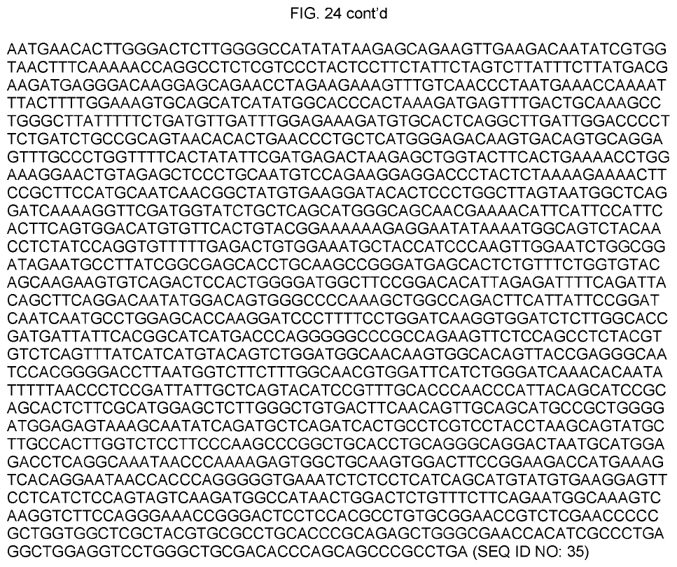

Visualization of hFVIII in UMGD treated liver tissue. To determine the cell type(s) transfected in the liver tissue following UMGD treatment under HE or LE condition, RNAscope and immunofluorescent staining was performed. Liver sections of HE & LE mice were collected on day 7 and day 120 and stained using RNAscope© protocols—in which DAPI mRNA is displayed in blue, hFVIII mRNA derived from the pUCOE-ICAM2-F8N6X10 in green, and Lyve-1 mRNA in red. LE & HE mice showed high levels of hFVIII staining on day 7 that decreased to lower levels on day 120-consistent with the aPTT data. However, differential transfections of LSECs vs hepatocytes were obtained by LE and HE US conditions, respectively. LE sections at both time points showed evidence of colocalization between the LSEC marker, Lyve-1, and hFVIII. This result indicates that LSECs were targeted with the combination of the pUCOE-ICAM2-F8N6X10 and LE US conditions to generate FVIII expression. The HE mice exhibited trace amounts of colocalization at both time-points, indicating a low level of LSEC transfection. Additional RNAscope staining performed with an albumin probe (a hepatic cell marker; FIG. 23A) showed hFVIII mRNA was found in hepatocytes in the HE treated mice, but not in the LE treated mice. RNAscope staining was also performed with a CD-31 probe (FIG. 23B). In LE mice, there were multiple regions of colocalization between hFVIII mRNA and CD-31, but trace amounts of colocalization in HE mice.

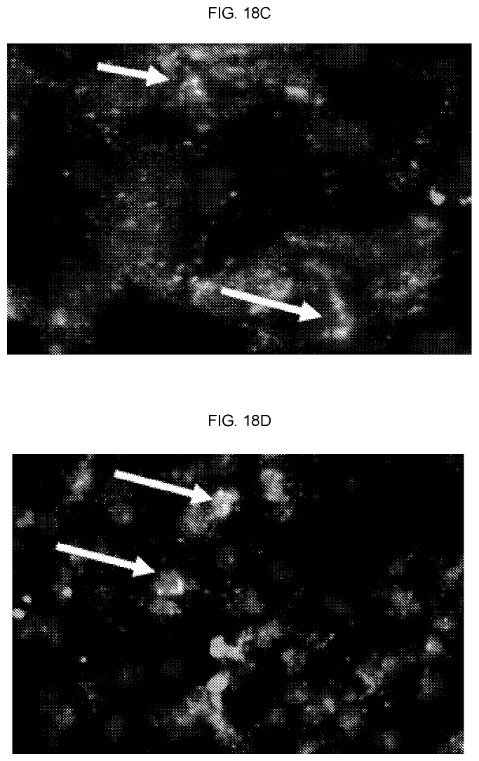

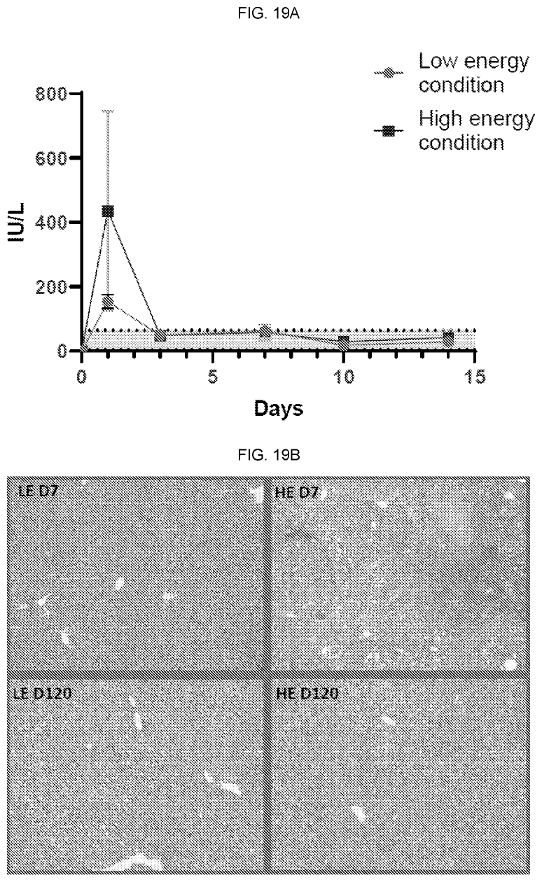

In addition, immunofluorescent staining of liver sections from treated mice on day 180 was performed using antibodies against endothelial cell marker CD-31 and hFVIII (FIG. 18B). The LE mouse sections showed high levels of hFVIII/CD-31 overlap, indicating hFVIII primarily was produced in endothelial cells. Similarly to what was seen in the RNAscope images, the HE mouse showed that most of the FVIII protein staining did not co-localize with CD-31 staining.

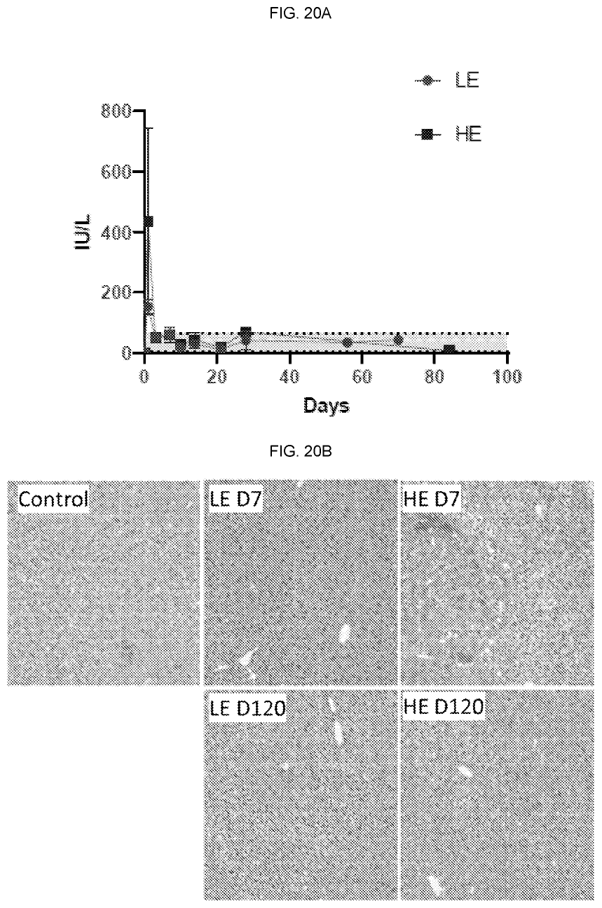

Examination of liver damage in UMGD mice. Levels of alanine transaminase (ALT) were measured to determine if UMGD induced any damage to the liver (FIG. 20A). When compared to untreated control mice (normal range: 18-65 IU/L), both the HE and LE groups generated elevated levels of ALT, 435 IU/L and 153 IU/L respectively, one day following surgery, indicating that the injection of MBs/plasmids and exposure to ultrasound induced transient minor damage and inflammation to the mouse liver. In addition, the LE US condition reduced the extent of transient damage compared to the HE condition. Furthermore, by day 3, both groups had returned to normal levels, 48 IU/L, which persisted for the remainder of the study, indicating there is no long-term damage to the liver.

In addition, livers from both groups of mice were collected at an early time-point, day 7, and a late time-point, day 120, to assess levels of damage through histology. The livers were fixed in 10% formalin, paraffinized, stained with hematoxylin & eosin (H&E), and imaged at 10× amplification. An untreated mouse was also sectioned and stained to act as a control. Representative images were selected to show the maximum extent of injury to the liver (FIG. 20B). On day 7, in a selected LE US-treated mouse liver section, there appeared to be small amounts of hemorrhage as well as light parenchymal necrosis, whereas more damages including hemorrhage, parenchymal necrosis, and possible fibrosis identified were detected in a selected HE US-treated mouse liver section. At the late time-point, the liver morphology in both the HE and LE mice returned to normal (FIG. 20B). These results are consistent with the transaminase examination that liver damages were reduced in LE mice compared to HE mice and no lasting damage was detected in both groups of mice.

### DISCUSSION

Gene therapy represents a highly promising alternative method to treat HA patients. While there were many advancements, a long-term effective, and non-immunogenic therapy remains elusive (Nguyen, G N, et al. (2021). Nat Biotechnol 39:47-55; Pipe, S W, et al. (2022). Expert Opin Biol Ther 22:1099-1115; Schutgens, REG (2022). Hemasphere 6: e720). Current gene therapy trials for HemA are mainly designed to introduce FVIII gene into hepatocytes using adeno-associated viral (AAV) vectors. Recent clinical trials (Rangarajan, S, et al. (2017). N Engl J Med 377:2519-2530; George, L A (2017). Blood Adv 1:2591-2599) have shown promising results. However, the immune responses to viral vectors, transduced cells, and transgene products, and potential tumor genesis (Hacein-Bey-Abina, S, et al. (2003). Science 302:415-419) remain hurdles to overcome for viral gene therapy (Herzog, R W (2015). Mol Ther 23:1411-1412). Furthermore, FVIII levels dropped precipitously over time in treated patients due to yet unknown reasons. Recent studies suggested that ectopic FVIII overexpression in hepatocytes may lead to cellular stress, resulting in apoptosis of transduced cells and toxicity, even tumor formation (Kapelanski-Lamoureux, A, et al. (2022). Mol Ther 30:3542-3551). In addition, it is currently impossible to administer repeated treatment in the presence of anti-AAV antibodies in previously treated patients. Compared to viral gene transfer, UMGD of pDNA elicits reduced immune responses and toxicity due to UMGD's specific targeting capacity, prevents random integration of DNA into the host genome (Nayak, S, and Herzog, R W (2010). Gene Ther 17:295-304), and allows for repeated delivery of the vectors. In addition, the pDNA cargo is relatively easier to prepare and more cost effective. Other nonviral gene delivery methods such as lipid nanoparticles recently showed high efficiency in delivering RNA and small molecules in vivo (An, D, et al. (2018). Cell Rep 24:2520; Cao, J, et al. (2019). Mol Ther 27:1242-1251; Hassett, K J, et al. (2019). Mol Ther Nucleic Acids 15:1-11), however were not capable of delivering DNA across the nuclear membrane efficiently for DNA transcription. UMGD of FVIII plasmids can bring significant benefit to a large population of HemA patients.

Liver sinusoidal endothelial cells (LSECs) are the primary natural cellular source of FVIII biosynthesis (Everett, L A, et al. (2014). Blood 123:3697-3705; Fahs, S A, et al. (2014). Blood 123:3706-3713). Gene therapy and transplantation studies to deliver FVIII gene into either hepatocytes (Greengard, J S, and Jolly, D J (1999). Thromb Haemost 82:555-561; Miao, C H, et al. (2006). Blood 108:19-27; Sarkar, R, et al. (2004). Blood 103:1253-1260; VandenDriessche, T, et al. (1999). Proc Natl Acad Sci USA 96:10379-10384) or endothelial cells (Kren, B T, et al. (2009). J Clin Invest 119:2086-2099; Matsui, H, et al. (2007). Stem Cells 25:2660-2669; Xu, L, et al. (2005). Proc Natl Acad Sci USA 102:6080-6085) or both (Yadav, N, et al. (2009). Blood 114:4552-4561) have improved HemA phenotype in mice. Nevertheless, ectopic expression of FVIII in other cell types such as hepatocytes can induce cellular stress responses and increase the risk of anti-FVIII inhibitor formation. (Brown, H C, et al. (2014). Mol Ther Methods Clin Dev 1:14036; Lytle, A M, et al. (2016). Mol Ther Methods Clin Dev 3:15056). Although FVIII mRNA has been found in several endothelial cell subsets including LSECs, lymphatic, and glomerular ECs (Hollestelle, M J, et al. (2001). Thromb Haemost 86:855-861; Shahani, T, et al. (2014). J Thromb Haemost 12:36-42; Do, H, et al. (1999). J Biol Chem 274:19587-19592; Pan, J, et al. (2016). Blood 128:104-109; Merlin, S, et al. (2019). Blood Adv 3:825-838), not all the endothelial cell subsets are suitable for production of FVIII. Previous studies indicated that transgene expression of FVIII in lung and heart endothelial cells induced strong anti-FVIII immune responses. (Merlin, S, et al. (2019). Blood Adv 3:825-838.) On the other hand, LSECs serve as resident antigen-presenting cells that are responsible for immune tolerance induction in the liver. Expression of FVIII in LSECs can promote antigen-specific tolerance due to its unique constitutive pro-tolerogenic properties. (Knolle, P A, and Wohlleber, D (2016). Cell Mol Immunol 13:347-353; Shetty, S, et al. (2018). Nat Rev Gastroenterol Hepatol 15:555-567; Doherty, DG (2019). Nat Biomed Eng 3:763-765; Shi, Q, et al. (2020). Blood Adv 4:2272-2285.)

Previous studies have shown that UMGD is a safe, well tolerated gene transfer method to deliver FVIII pDNA to HA animals with success (Song, S, et al. (2022). Mol Ther Nucleic Acids 27:916-926). However, past studies have primarily focused on delivery of FVIII gene into hepatic cells. In this experimental example, UMGD was described in combination with an endothelial specific plasmid to target FVIII expression in LSECs to enhance the safety and feasibility for long-term correction of HemA. Previous UMGD studies with a luciferase plasmid in mice, showed US condition pairings that were between 1J and 10J of energy resulted in the highest levels of transfection with acceptably low levels of damage (Tran, D M, et al. (2018). J Control Release 279:345-354). In this experimental example, several conditions fell along the curve of highest transfection from these original experiments were selected to compare transfection patterns in the GFP experiments. Additional conditions were chosen below this energy curve of high transfection. This resulted in a total matrix of US conditions ranging from 0.5-2.5 MPa peak negative pressure (PNP) and 18 μs to 22 ms pulse duration (PD) in combination with 1.1 MHz frequency and 14 Hz PRF. While most of the conditions resulted in GFP expression in hepatocytes or a mix of hepatocytes and LSECs, one condition, 50 W/cm2, 150 μs PD, showed strong evidence of predominately LSEC transfection and was chosen for further testing in the hFVIII pDNA experiments. In this experimental example, LSECs displayed high levels of transfection at lower levels of power than hepatocytes.

When delivering the hFVIII pDNA to the experimental HA mice, in addition to the LSEC targeting US condition, a hepatocyte targeting US condition was tested to act as a comparison for FVIII activity and safety of the procedure. In the LE mouse group, improved FVIII activity was sustained over 84 days and was comparable to the HE mouse group. In this experimental example, while the HE condition primarily transfected hepatocytes, LSECs are also transfected. This is supported by small regions of colocalization between the FVIII and endothelial cell markers in both types of staining done with the HE mice. The HE condition appears to be less specific in the cell type transfected, while the LE US wave only provides energy enough for the interaction to occur in the region of endothelial cells. When comparing the two groups, there was a difference in the lack of anti-FVIII inhibitors found in the LE mice. Arguably one of the largest issues plaguing the treatment of HA is the formation of inhibitors (Haya, S, et al. (2007). Haemophilia 13 Suppl 5:52-60), which complicates future treatment and can drastically reduce FVIII activity. The treatment of HE and LE groups was identical apart from the US power used, leading to differentially dominant hepatocyte and endothelial cell transfections, respectively. Based on the results in this experimental example, transgene expression in LSECs more favorably facilitates the induction of antigen-specific tolerance due to its unique constitutive pro-tolerogenic properties. (Knolle, P A, and Wohlleber, D (2016). Cell Mol Immunol 13:347-353; Shetty, S, et al. (2018). Nat Rev Gastroenterol Hepatol 15:555-567; Doherty, D G (2019). Nat Biomed Eng 3:763-765; Shi, Q, et al. (2020). Blood Adv 4:2272-2285).

The ability to target the primary production site of FVIII at a lower power of US is beneficial for multiple reasons. Unlike other forms of ultrasound, such as diagnostic imaging, the power associated with UMGD treatment is much higher, creating more heat accumulation or cavitation-induced tissue damage. Thus, the lower the power needed to achieve successful transfection also results in lower tissue damage. This is supported when comparing both the ALT and histology data between the HE and LE groups of treated mice. While this experimental example showed a transient increase in ALT immediately following surgery in both groups, the damage caused by the LE condition is less substantial than that observed with the HE mice, indicating this condition is safer than that needed to target hepatocytes. In both energy condition groups, long-lasting damage was not observed, demonstrating the overall safety of this procedure in HA mice that are more prone to prolonged bleeding and hepatic apoptosis. Additionally, despite the inherent bleeding risk of performing a laparotomy on mice with HA, most animals that underwent a successful surgery returned to typical activity levels within 24 hours of the procedure. There is also the consideration with the development of an ultrasound transducer to perform UMGD depending on the desired power capacity. To output higher levels of power, transducers need to have more robust elements in terms of make and number, which may exceed the limit of the transducer element output capacity or translate to higher costs of specialized transducer design. By utilizing a lower power, effective transducers will be more easily designed and cheaper, or even commercially produced transducers can be used.

This experimental example describes the ability to target the native production site of FVIII, LSECs, through the non-viral method of UMGD. Through initial experiments testing the transfection patterns of a GFP reporter plasmid with varying US conditions, specific US parameters were defined that could be used to transfect different dominant cell types. The ability to target specific cell types through alterations of US parameters in combination with the programmable nature of plasmids extends the variety of genetic diseases UMGD technology can be applied to as well. In this experimental example, with selected ultrasound parameters targeting LSECs, persistent therapeutic levels of FVIII were achieved without the formation of anti-FVIII inhibitory antibodies in immunocompetent HA mice. FVIII synthesized in LSECs immediately complexes with vWF, and therefore is better protected and can be functionally more stable for long-term expression. Furthermore, lower US energy requirement for transecting LSECs is beneficial in several fronts including overcoming the constraints of the upper limits of the transducer materials and US parameter boundaries, as well as decreasing any potential transient damage to the treatment tissue. This experimental example describes targeting LSECs with UMGD is a safe and non-immunogenic method of delivering FVIII gene, which can facilitate in creating a widely applicable, long-term, HA treatment.

(x) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant increase in an immune response to variant Factor VIII.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A factor VIII protein comprising a deletion of glycans at residue 2118 within the C1 domain.

2. A factor VIII protein comprising a mutation at residue 2118, wherein the mutation reduces or eliminates glycosylation at residue 2118.

3. The factor VIII protein of claim 2, wherein the mutation comprises an asparagine to glutamine mutation.

4. The factor VIII protein of claim 2, further comprising a B domain deletion (BDD-FVIII) or a B domain truncation.

5. The factor VIII protein of claim 4, wherein the B domain truncation comprises a truncated B domain having 226 amino acids and only 6 N-linked glycosylation sites (N6) or a truncated B domain having 17 amino acids (V3).

6. The factor VIII protein of claim 2, further comprising a mutation in the B domain at residue 1645.

7. The factor VIII protein of claim 6, wherein the mutation in the B domain comprises an arginine to histidine mutation.

8. The factor VIII protein of claim 2, further comprising a mutation in the A1 domain.

9. The factor VIII protein of claim 8, comprising V861, F105Y, S108A, E115D, H117Q, L129F, K132G, Q134H, T147M, and P152L mutations in the A1 domain (X10).

10. The factor VIII protein of claim 8, wherein the mutation in the A1 domain comprises F309S.

11. The factor VIII protein of claim 2, further comprising a furin-cleavage site deletion.

12. The factor VIII protein of claim 2, further comprising mutations in the C1 and C2 domains.

13. The factor VIII protein of claim 12, comprising V18571, H1859R, M1907K, M1926K, L1975V, A1993V, H2007Q, D2066E, K2085M, Q2113H, S2157N, and R2159H mutations (K12).

14. The factor VIII protein of claim 2, wherein the factor VIII protein comprises a BDD-FVIII-N2118Q, BDD-FVIII*-N2118Q, F8/N6-N2118Q, F8/V3-N2118Q, BDD-FVIII-RH-N2118Q, BDD-FVIII*-RH-N2118Q, F8/N6RH-N2118Q, F8/V3RH-N2118Q, BDD-FVIII-X10-N2118Q, F8/N6-X10-N2118Q, BDD-FVIII*-X10-N2118Q, F8/V3X10-N2118Q, BDD-FVIII-X10-RH-N2118Q, BDD-FVIII*-X10-RH-N2118Q, F8/N6-X10-RH-N2118Q, F8/V3-X10-RH-N2118Q, BDD-FVIII-F309S-N2118Q, BDD-FVIII*-F309S-N2118Q, F8/N6F309S-N2118Q, F8/V3F309S-N2118Q, BDD-FVIII-F309S-N2118Q-RH, BDD-FVIII*-F309S-N2118Q-RH, F8/N6-F309S-N2118Q-RH, F8/V3-F309S-N2118Q-RH, BDD-FVIII-K12-N2118Q, BDD-FVIII*-K12-N2118Q, F8/N6K12-N2118Q, F8/V3K12-N2118Q, BDD-FVIII-K12-N2118Q-RH, BDD-FVIII*-K12-N2118Q-RH, F8/N6-K12-N2118Q-RH, F8/V3-K12-N2118Q-RH, BDD-FVIII-X10-F309S-N2118Q, BDD-FVIII*-X10-F309S-N2118Q, F8/N6X10-F309S-N2118Q, F8/V3X10-F309S-N2118Q, BDD-FVIII-X10-F309S-N2118Q-RH, BDD-FVIII*-X10-F309S-N2118Q-RH, F8/N6-X10-F309S-N2118Q-RH, F8/V3-X10-F309S-N2118Q-RH, BDD-FVIII-X10-K12-N2118Q, BDD-FVIII*-X10-K12-N2118Q, F8/N6X10-K12-N2118Q, F8/V3X10-K12-N2118Q, BDD-FVIII-X10-K12-N2118Q-RH, BDD-FVIII*-X10-K12-N2118Q-RH, F8/N6-X10-K12-N2118Q-RH, F8/V3-X10-K12-N2118Q-RH, BDD-FVIII-K12-F309S-N2118Q, BDD-FVIII*-K12-F309S-N2118Q, F8/N6K12-F309S-N2118Q, F8/V3K12-F309S-N2118Q, BDD-FVIII-K12-F309S-N2118Q-RH, BDD-FVIII*-K12-F309S-N2118Q-RH, F8/N6-K12-F309S-N2118Q-RH, F8/V3-K12-F309S-N2118Q-RH, BDD-FVIII-X10-K12-F309S-N2118Q, BDD-FVIII*-X10-K12-F309S-N2118Q, F8/N6X10-K12-F309S-N2118Q, F8/V3X10-K12-F309S-N2118Q, BDD-FVIII-X10-K12-F309S-N2118Q-RH, BDD-FVIII*-X10-K12-F309S-N2118Q-RH, F8/N6-X10-K12-F309S-N2118Q-RH, or F8/V3-X10-K12-F309S-N2118Q-RH, wherein * indicates a furin cleavage site deletion.

15. The factor VIII protein of claim 2, wherein the factor VIII protein comprises BDD-FVIII-N2118Q, BDD-FVIII-X10-N2118Q, BDD-F8/N6-N2118Q, BDD-F8/N6-X10-N2118Q, or BDD-CF8-X10-N2118Q.

16. The factor VIII protein of claim 15, wherein the factor VIII protein is encoded by the sequence as set forth in SEQ ID NOs: 23, 27, 31, 34, or 35 or a sequence having at least 90% sequence identity to the sequence as set forth in SEQ ID NOs: 23, 27, 31, 34, or 35.

17. A nucleic acid encoding a factor VIII protein comprising a deletion of glycans at residue 2118 within the C1 domain.

18. A nucleic acid encoding a factor VIII protein of claim 2.

19. The nucleic acid of claim 18, having the sequence as set forth in SEQ ID NOs: 23, 27, 31, 34, or 35 or a sequence having at least 90% sequence identity to the sequence as set forth in SEQ ID NOs: 23, 27, 31, 34, or 35.

20. The nucleic acid of claim 18, having the sequence as set forth in SEQ ID NO: 21, 22, 24-26, 28-30, 32, or 33 with nucleotide substitutions to generate an N2118Q mutation.

21. The nucleic acid of claim 20, wherein the nucleotide substitutions comprise replacing AAT or AAC with CAA or CAG.

22. The nucleic acid of claim 18, within a genetic construct comprising a promoter operably linked to the nucleic acid.

23. The nucleic acid of claim 22, wherein the genetic construct further comprises an enhancer operably linked to the nucleic acid.

24. The nucleic acid of claim 22, wherein the genetic construct further comprises a 3′UTR.

25. The nucleic acid of claim 23, wherein the enhancer comprises a ubiquitous chromatin opening element (UCOE) enhancer or a hepatic control region (HCR).

26. The nucleic acid of claim 22, wherein the promoter comprises a liver sinusoidal endothelial cell (LSEC)-specific promoter.

27. The nucleic acid of claim 26, wherein the LSEC-specific promoter comprises ICAM2, Stabilin-2, Tie2, Flk-1, or VE cadherin.

28. The nucleic acid of claim 22, wherein the promoter comprises a hepatocyte-specific promoter.

29. The nucleic acid of claim 28, wherein the hepatocyte-specific promoter comprises an hAAT promoter.

30. The nucleic acid of claim 22, wherein the promoter comprises a ubiquitous promoter.

31. The nucleic acid of claim 30, wherein the ubiquitous promoter comprises an SV40 promoter, CMV promoter, PGK promoter, or CAG promoter.

32. The nucleic acid of claim 24, wherein the 3′UTR comprises an miRT-122 or an miRT-142-3p.

33. The nucleic acid of claim 22, wherein the genetic construct further comprises a nuclear localization signal.

34. The nucleic acid of claim 33, wherein the nuclear localization signal comprises an SV40 nuclear localization signal.

35. The nucleic acid of claim 18, within a vector for delivery to a cell.

36. The nucleic acid of claim 35, wherein the vector is a viral vector.

37. The nucleic acid of claim 36, wherein the viral vector is a lentiviral vector.

38. The nucleic acid of claim 36, wherein the viral vector is an adeno-associated viral vector (AAV).

39. The nucleic acid of claim 22, wherein the genetic construct further comprises homology arms.

40. The nucleic acid of claim 39, wherein the homology arms are homologous to an endogenous factor VIII locus.

41. The nucleic acid of claim 39, wherein the homology arms are homologous to a site within a genomic safe harbor.

42. The nucleic acid of claim 18, wherein the nucleic acid comprises cDNA.

43. A nanoparticle comprising the nucleic acid of claim 18.

44. A composition comprising (i) a factor VIII protein of claim 2, a nucleic acid of claim 18, and/or a nanoparticle of claim 43 and (ii) a pharmaceutically acceptable carrier.

45. A method of treating a subject for hemophilia, the method comprising administering a therapeutically effective amount of the composition of claim 44 to the subject, thereby treating the subject for the hemophilia.

46. The method of claim 45, wherein the hemophilia is hemophilia A.

47. The method of claim 45, wherein the administering comprises intraosseous, intravenous, intradermal, intraarterial, intranodal, intralymphatic, intraperitoneal, intralesional, intraprostatic, intravaginal, intrarectal, intrathecal, intramuscular, intravesicular, and/or subcutaneous administration.

48. The method of claim 47, wherein the intravenous administration comprises portal vein injection.

49. The method of claim 45, wherein the administering comprises intraosseous administration.

50. The method of claim 45, wherein the administering utilizes ultrasound.

51. The method of claim 45, wherein the therapeutically effective amount has lowered immunogenicity as compared to native FVIII.

52. A method for expressing a genetic construct encoding a factor VIII protein within a population of cells, the method comprising administering the nucleic acid of claim 18 and/or the nanoparticle of claim 43 in a sufficient dosage and for a sufficient time to the population of cells thereby expressing the genetic construct within the population of cells.

53. The method of claim 52, wherein the population of cells is in vivo at the time of the administering.

54. The method of claim 52, wherein the cells are ex vivo at the time of the administering.

55. The method of claim 52, wherein the population of cells comprise liver sinusoidal endothelial cells (LSECs).

56. The method of claim 52, wherein the administering comprises ultrasound-mediated gene delivery (UMGD).

57. The method of claim 56, wherein the UMGD utilizes a microbubble or a nanobubble.

58. The method of claim 57, wherein the microbubble has a diameter in a range of 0.2-3 microns (μm).

59. The method of claim 57, wherein the microbubble has an average diameter of 1 micron.

60. The method of claim 57, wherein the microbubble or the nanobubble is delivered intravenously.

61. The method of claim 56, wherein the UMGD utilizes transcutaneous ultrasound.

62. The method of claim 56, wherein the UMGD utilizes a peak negative pressure in the range of 0.5-2.5 megapascals (MPa).

63. The method of claim 56, wherein the UMGD utilizes a pulse duration in the range of 18-2000 microseconds (μs).

64. The method of claim 56, wherein the UMGD utilizes a frequency in a range of 0.8-1.4 megahertz (MHz).

65. The method of claim 56, wherein the UMGD utilizes a frequency of 1.1 MHz.

66. The method of claim 56, wherein the UMGD utilizes a pulse repetition frequency (PRF) in a range of 1-50 Hertz (Hz).

67. The method of claim 56, wherein the UMGD utilizes a PRF of 14 Hz.

68. The method of any claim 56, wherein the UMGD utilizes an intensity of 0-75 W/cm².

69. The method of claim 56, wherein the UMGD utilizes an intensity of 50 W/cm² and a pulse duration of 150 μs.

70. The method of claim 56, wherein the UMGD utilizes an intensity of 76-200 W/cm².

71. The method of claim 56, wherein the UMGD utilizes an intensity of 110 W/cm² and a pulse duration of 150 μs.

72. The method of claim 56, wherein the UMGD results in preferential delivery of the nucleic acid or nanoparticle to epithelial cells over hepatocytes.

73. The method of claim 52, wherein the administering comprises intraosseous, intravenous, intradermal, intraarterial, intranodal, intralymphatic, intraperitoneal, intralesional, intraprostatic, intravaginal, intrarectal, intrathecal, intramuscular, intravesicular, and/or subcutaneous administration.

74. The method of claim 73, wherein the intravenous administration comprises portal vein injection.

75. The method of claim 52, wherein the administering comprises intraosseous administration.

76. The method of claim 52, wherein the subject is a human, mouse, canine, or non-human primate.

77. The method of claim 52, wherein the administering comprises pipetting.


SEQUENCE LISTING (35 sequences)

SEQ ID NO: 1 - DNA, 49 bp, synthetic construct
  source 1..49: mol_type = other DNA
gagtgccaaaatcttttccattccaaacctcagtcgtgtacaaaaagac

SEQ ID NO: 2 - DNA, 49 bp, synthetic construct
  source 1..49: mol_type = other DNA
gtctttttgtacacgactgaggtttggaatggaaaagattttggcactc

SEQ ID NO: 3 - DNA, 44 bp, synthetic construct
  source 1..44: mol_type = other DNA
cacagtcaatggttatgtacaaaggtctctgccaggtctgattg

SEQ ID NO: 4 - DNA, 44 bp, synthetic construct
  source 1..44: mol_type = other DNA
caatcagacctggcagagacctttgtacataaccattgactgtg

SEQ ID NO: 5 - DNA, 44 bp, synthetic construct
  source 1..44: mol_type = other DNA
ctttgtcaagcctcaggaaaccaaaacttacttttggaaagtgc

SEQ ID NO: 6 - DNA, 44 bp, synthetic construct
  source 1..44: mol_type = other DNA
gcactttccaaaagtaagttttggtttcctgaggcttgacaaag

SEQ ID NO: 7 - DNA, 41 bp, synthetic construct
  source 1..41: mol_type = other DNA
gtggcagacttatcgaggacaatccactggaaccttaatgg

SEQ ID NO: 8 - DNA, 41 bp, synthetic construct
  source 1..41: mol_type = other DNA
ccattaaggttccagtggattgtcctcgataagtctgccac

SEQ ID NO: 9 - DNA, 20 bp, synthetic construct
  source 1..20: mol_type = other DNA
tgaccctgaagttcatctgc

SEQ ID NO: 10 - DNA, 20 bp, synthetic construct
  source 1..20: mol_type = other DNA
gaagtcgtgctgcttcatgt

SEQ ID NO: 11-12 - skipped (2 entries)

SEQ ID NO: 13 - protein, 2351 aa, Homo sapiens [sequence omitted]

SEQ ID NO: 14 - protein, 2319 aa, Mus musculus
MQIALFACFFLSLFNFCSSAIRRYYLGAVELSWNYIQSDLLSVLHTDSRFLPRMSTSFPFNTSIMYKKTVFVEYKDQLFNIAKPRPPWMGLLGPTIWTEVHDTVVITLKNMASHPVSLHAVGVSYWKASEGDEYEDQTSQMEKEDDKVFPGESHTYVWQVLKENGPMASDPPCLTYSYMSHVDLVKDLNSGLIGALLVCKEGSLSKERTQMLYQFVLLFAVFDEGKSWHSETNDSYTQSMDSASARDWPKMHTVNGYVNRSLPGLIGCHRKSVYWHVIGMGTTPEIHSIFLEGHTFFVRNHRQASLEISPITFLTAQTLLIDLGQFLLFCHISSHKHDGMEAYVKVDSCPEESQWQKKNNNEEMEDYDDDLYSEMDMFTLDYDSSPFIQIRSVAKKYPKTWIHYISAEEEDWDYAPSVPTSDNGSYKSQYLSNGPHRIGRKYKKVRFIAYTDETFKTRETIQHESGLLGPLLYGEVGDTLLIIFKNQASRPYNIYPHGITDVSPLHARRLPRGIKHVKDLPIHPGEIFKYKWTVTVEDGPTKSDPRCLTRYYSSFINPERDLASGLIGPLLICYKESVDQRGNQMMSDKRNVILFSIFDENQSWYITENMQRFLPNAAKTQPQDPGFQASNIMHSINGYVFDSLELTVCLHEVAYWHILSVGAQTDFLSIFFSGYTFKHKMVYEDTLTLFPFSGETVFMSMENPGLWVLGCHNSDFRKRGMTALLKVSSCDKSTSDYYEEIYEDIPTQLVNENNVIDPRSFFQNTNHPNTRKKKFKDSTIPKNDMEKIEPQFEEIAEMLKVQSVSVSDMLMLLGQSHPTPHGLFLSDGQEAIYEAIHDDHSPNAIDSNEGPSKVTQLRPESHHSEKIVFTPQPGLQLRSNKSLETTIEVKWKKLGLQVSSLPSNLMTTTILSDNLKATFEKTDSSGFPDMPVHSSSKLSTTAFGKKAYSLVGSHVPLNVSEENSDSNILDSTLMYSQESLPRDNILSMENDRLLREKRFHGIALLTKDNTLFKDNVSLMKTNKTYNHSTTNEKLHTESPTSIENSTTDLQDAILKVNSEIQEVTALIHDGTLLGKNSTYLRLNHMLNRTTSTKNKDIFHRKDEDPIPQDEENTIMPFSKMLFLSESSNWFKKTNGNNSLNSEQEHSPKQLVYLMFKKYVKNQSFLSEKNKVTVEQDGFTKNIGLKDMAFPHNMSIFLTTLSNVHENGRHNQEKNIQEEIEKEALIEEKVVLPQVHEATGSKNFLKDILILGTRQNISLYEVHVPVLQNITSINNSTNTVQIHMEHFFKRRKDKETNSEGLVNKTREMVKNYPSQKNITTQRSKRALGQFRLSTQWLKTINCSTQCIIKQIDHSKEMKKFITKSSLSDSSVIKSTTQTNSSDSHIVKTSAFPPIDLKRSPFQNKFSHVQASSYIYDFKTKSSRIQESNNFLKETKINNPSLAILPWNMFIDQGKFTSPGKSNTNSVTYKKRENIIFLKPTLPEESGKIELLPQVSIQEEEILPTETSHGSPGHLNLMKEVFLQKIQGPTKWNKAKRHGESIKGKTESSKNTRSKLLNHHAWDYHYAAQIPKDMWKSKEKSPEIISIKQEDTILSLRPHGNSHSIGANEKQNWPQRETTWVKQGQTQRTCSQIPPVLKRHQRELSAFQSEQEATDYDDAITIETIEDFDIYSEDIKQGPRSFQQKTRHYFIAAVERLWDYGMSTSHVLRNRYQSDNVPQFKKVVFQEFTDGSFSQPLYRGELNEHLGLLGPYIRAEVEDNIMVTFKNQASRPYSFYSSLISYKEDQRGEEPRRNFVKPNETKIYFWKVQHHMAPTEDEFDCKAWAYFSDVDLERDMHSGLIGPLLICHANTLNPAHGRQVSVQEFALLFTIFDETKSWYFTENVKRNCKTPCNFQMEDPTLKENYRFHAINGYVMDTLPGLVMAQDQRIRWYLLSMGNNENIQSIHFSGHVFTVRKKEEYKMAVYNLYPGVFETLEMIPSRAGIWRVECLIGEHLQAGMSTLFLVYSKQCQIPLGMASGSIRDFQITASGHYGQWAPNLARLHYSGSINAWSTKEPFSWIKVDLLAPMIVHGIKTQGARQKFSSLYISQFIIMYSLDGKKWLSYQGNSTGTLMVFFGNVDSSGIKHNSFNPPIIARYIRLHPTHSSIRSTLRMELMGCDLNSCSIPLGMESKVISDTQITASSYFTNMFATWSPSQARLHLQGRTNAWRPQVNDPKQWLQVDLQKTMKVTGIITQGVKSLFTSMFVKEFLISSSQDGHHWTQILYNGKVKVFQGNQDSSTPMMNSLDPPLLTRYLRIHPQIWEHQIALRLEILGCEAQQQY

SEQ ID NO: 15 - protein, 15 aa, synthetic construct
KWQTYRGNSTGTLMV

SEQ ID NO: 16 - protein, 15 aa, synthetic construct
KWQTYRGNSTGTLMV

SEQ ID NO: 17 - protein, 15 aa, synthetic construct
TYRGNSTGTLMVFFG

SEQ ID NO: 18 - protein, 15 aa, synthetic construct
DGKKWQTYRGNSTGT

SEQ ID NO: 19 - protein, 7 aa, synthetic construct
PKKKRKV

SEQ ID NO: 20 - protein, 14 aa, synthetic construct
RPAATKKAGQAKKK

SEQ ID NO: 21 - DNA, 4416 bp, synthetic construct
  source 1..4416: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 22 - DNA, 4474 bp, synthetic construct
  source 1..4474: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttttctgatgttgatttggagaaagatgtgcactcaggcttgattggaccccttctgatctgccgcactaacacactgaaccctgctcatgggagacaagtgacagtacaggaatttgctctgtttttcaccatctttgatgagaccaaaagctggtacttcactgaaaatatggaaagaaactgcagggctccctgcaatatccagaaggaggatcccacttttaaagagaattatcgcttccatgcaatcaacggctatataaaggatacactccctggcttagtaatggctcaggatcaaaggattcgatggtatctgctcagcatgggcagcaatgaaaacatccattctattcatttcagtggacatgtgttcactgtacggaaaaaagaggaatataaaatggcagtctacaacctctatccaggtgtttttgagactgtggaaatgctaccatccaaagttggaatctggcgggtggaatgccttatcggcgagcacctgcaagccgggatgagcactctgtttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacatctctcagtttatcatcatgtatagtcttgatggcaagaagtggcacacttaccgaggaaattccactggaaccttaatggtcttctttggcaatgtggattcatctgggataaaacacaatatttttaaccctccaattattgctcgatacatccgtttgcacccaactcattataacattcacagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagctcatggcgcgcctaggccttgacggccttccgccaattcgccggatcc

SEQ ID NO: 23 - DNA, 4416 bp, synthetic construct
  source 1..4416: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggacaatccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 24 - DNA, 4359 bp, synthetic construct
  source 1..4359: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaccaccagtcttgaaacaccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 25 - DNA, 4425 bp, synthetic construct
  source 1..4425: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 26 - DNA, 4419 bp, synthetic construct
  source 1..4419: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 27 - DNA, 4419 bp, synthetic construct
  source 1..4419: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggacaatccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 28 - DNA, 5040 bp, synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 29 - DNA, 5095 bp, synthetic construct
  source 1..5095: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttttctgatgttgatttggagaaagatgtgcactcaggcttgattggaccccttctgatctgccgcactaacacactgaaccctgctcatgggagacaagtgacagtacaggaatttgctctgtttttcaccatctttgatgagaccaaaagctggtacttcactgaaaatatggaaagaaactgcagggctccctgcaatatccagaaggaggatcccacttttaaagagaattatcgcttccatgcaatcaacggctatataaaggatacactccctggcttagtaatggctcaggatcaaaggattcgatggtatctgctcagcatgggcagcaatgaaaacatccattctattcatttcagtggacatgtgttcactgtacggaaaaaagaggaatataaaatggcagtctacaacctctatccaggtgtttttgagactgtggaaatgctaccatccaaagttggaatctggcgggtggaatgccttatcggcgagcacctgcaagccgggatgagcactctgtttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacatctctcagtttatcatcatgtatagtcttgatggcaagaagtggcacacttaccgaggaaattccactggaaccttaatggtcttctttggcaatgtggattcatctgggataaaacacaatatttttaaccctccaattattgctcgatacatccgtttgcacccaactcattataacattcacagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagctcatggcgcgcctaggccttgacggccttccgccaattcgccggatcc

SEQ ID NO: 30 - DNA, 5095 bp, synthetic construct
  source 1..5095: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacaccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttttctgatgttgatttggagaaagatgtgcactcaggcttgattggaccccttctgatctgccgcactaacacactgaaccctgctcatgggagacaagtgacagtacaggaatttgctctgtttttcaccatctttgatgagaccaaaagctggtacttcactgaaaatatggaaagaaactgcagggctccctgcaatatccagaaggaggatcccacttttaaagagaattatcgcttccatgcaatcaacggctatataaaggatacactccctggcttagtaatggctcaggatcaaaggattcgatggtatctgctcagcatgggcagcaatgaaaacatccattctattcatttcagtggacatgtgttcactgtacggaaaaaagaggaatataaaatggcagtctacaacctctatccaggtgtttttgagactgtggaaatgctaccatccaaagttggaatctggcgggtggaatgccttatcggcgagcacctgcaagccgggatgagcactctgtttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacatctctcagtttatcatcatgtatagtcttgatggcaagaagtggcacacttaccgaggaaattccactggaaccttaatggtcttctttggcaatgtggattcatctgggataaaacacaatatttttaaccctccaattattgctcgatacatccgtttgcacccaactcattataacattcacagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagctcatggcgcgcctaggccttgacggccttccgccaattcgccggatcc

SEQ ID NO: 31 - DNA, 5040 bp, synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggacaatccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 32 - DNA, 5040 bp, synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacaccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 33 - DNA, 5040 bp, synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 34 - DNA, 5040 bp, synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggacaatccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 35 - DNA, 4674 bp, synthetic construct
  source 1..4674: mol_type = other DNA
atgcaagtagagctctacacctgctgctttctgtgccttttgcccttcagccttagtgccaccagaaaatactacctcggtgcagtggaactgtcctgggactatatgcaaagtgacctgctcagtgcgctgcacgcggatacaagcttttcttccagggtgccaggatctttgccactcaccacgtcagtcacgtacagaaagactgtgtttgtagagtttacagatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctacgcgtgcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgtcttgtcatatctcttcccaccaacatgatggcatggaagcttatgtcaaagtagacagctgtccagaggaaccccaactgcgcatgaaaaataatgaagataaagattatgatgatggtctttatgattctgacatggacgtagttagctttgatgacgacagctcttctccctttatccaaatccgctcagttgccaagaggcatcctaaaacttgggtccactatattgctgctgaggaggaggactgggactatgctccctcaggccccacccccaatgatagaagtcataaaaatctgtatttgaacaatggtcctcagcggattggtaagaagtacaaaaaagtccgatttgtggcatacacagatgagacatttaagactcgtgaagctattcagtatgaatcaggaatcctgggacctttactttatggagaagttcatgggatcaattatgtcactcctctgcacacagggagattgccaaaaggtgtgaaacatttgaaagatatgccaattctgccgggagagatattcaagtataaatggatagtgaccgtagaagatggaccaactaaatcagatcctcggtgcctgacccgatattactcaagcttcattaatctggagagagatctagcttcaggactcattggccctcttctcatctgctacaaagaatctgtagatcaaagaggaaaccagatgatggcagacaagagaaatgtcatcctgttttctgtatttgatgagaatcgaagctggtacctcacagagaatagtcagcgcttcctccccaatgcagatgtagtgcagccccatgacccagagttccaactctctaacatcatgcacagcatcaatggctatgtttttgacaacttgcagctgtcagtttgtttgcatgaggtggcgtactggtacattctaagtgttggagcacaaactgacttcctgtctgtcttcttctctggatataccttcaaacacaaaatggtctatgaagacacacttaccctcttcccattctcaggagaaactgtcttcatgtcaatggaaaacccaggtctgtgggttctggggtgccacaactcagactttcggaacagaggcatgacagccttactgaaggtttctagttgtaacaggaacattgatgattattatgaggacacatacgaagatattccaactcccctgctaaatgaaaacaatgtaattaaacctagaagcttctcccagaattcaaggcaccctagcactaaggaaaagcaattgaaagccaccacaactccagaaaatgacatagagaagattgaccttcaatctggagaaagaacacagctgattaaagcacaaagtgtctcctctagtgatttgttgatgctgttgggacagaatcctactccacgtggactgttcttatctgatctccgagaggccacagatagagccgatgaccattcacgtggagcaatagaaagaaacaagggcccttgtgaaaataatgattcaacagcagcaataaatgaaggacaagataagccccaaagagaagctatgtgggcaaagcaaggagagcctggaaggttgtgctctcaaaacccaccagtctcaaaacaccatcaaagggaaataaccgttactactcttcagccagaggaagacaaatttgagtatgatgacaccttctcaattgaaatgaagagagaagattttgacatctacggcgactatgaaaatcagggcctccgcagctttcaaaagaaaacacgacactatttcattgctgcagtggagcgtctctggcattatgggatgagtagatctccccatatactaagaaacagggctcaaagtggggatgtccagcagttcaagaaggtggttttccaggaatttactgatggatcctttactcagcccttataccgtggaggactgaatgaacacttgggactcttggggccatatataagagcagaagttgaagacaatatcgtggtaactttcaaaaaccaggcctctcgtccctactccttctattctagtcttatttcttatgacgaagatgagggacaaggagcagaacctagaagaaagtttgtcaaccctaatgaaaccaaaatttacttttggaaagtgcagcatcatatggcacccactaaagatgagtttgactgcaaagcctgggcttatttttctgatgttgatttggagaaagatgtgcactcaggcttgattggaccccttctgatctgccgcagtaacacactgaaccctgctcatgggagacaagtgacagtgcaggagtttgccctggttttcactatattcgatgagactaagagctggtacttcactgaaaacctggaaaggaactgtagagctccctgcaatgtccagaaggaggaccctactctaaaagaaaacttccgcttccatgcaatcaacggctatgtgaaggatacactccctggcttagtaatggctcaggatcaaaaggttcgatggtatctgctcagcatgggcagcaacgaaaacattcattccattcacttcagtggacatgtgttcactgtacggaaaaaagaggaatataaaatggcagtctacaacctctatccaggtgtttttgagactgtggaaatgctaccatcccaagttggaatctggcggatagaatgccttatcggcgagcacctgcaagccgggatgagcactctgtttctggtgtacagcaagaagtgtcagactccactggggatggcttccggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacgtgtctcagtttatcatcatgtacagtctggatggcaacaagtggcacagttaccgagggcaatccacggggaccttaatggtcttctttggcaacgtggattcatctgggatcaaacacaatatttttaaccctccgattattgctcagtacatccgtttgcacccaacccattacagcatccgcagcactcttcgcatggagctcttgggctgtgacttcaacagttgcagcatgccgctggggatggagagtaaagcaatatcagatgctcagatcactgcctcgtcctacctaagcagtatgcttgccacttggtctccttcccaagcccggctgcacctgcagggcaggactaatgcatggagacctcaggcaaataacccaaaagagtggctgcaagtggacttccggaagaccatgaaagtcacaggaataaccacccagggggtgaaatctctcctcatcagcatgtatgtgaaggagttcctcatctccagtagtcaagatggccataactggactctgtttcttcagaatggcaaagtcaaggtcttccagggaaaccgggactcctccacgcctgtgcggaaccgtctcgaacccccgctggtggctcgctacgtgcgcctgcacccgcagagctgggcgaaccacatcgccctgaggctggaggtcctgggctgcgacacccagcagcccgcctga